(19) United States
(12) Patent Application Publication
(10) Pub. No.: US 2026/0327585 A1
(43) Pub. Date: Oct. 22, 2026

(54) COMPACT PROTEINS

(71) Applicant: VERTEX PHARMACEUTICALS INCORPORATED, Boston, MA (US)

(72) Inventors: Gregoriy A. DOKSHIN, Boston, MA (US); Michelle GOOD, Boston, MA (US); D'anna M. NELSON, Boston, MA (US); Darin Katsumi TAKEMOTO, Boston (UA); Sara Elizabeth SWIFT, Boston, MA (US); Jishnu SAHA, Boston, MA (US); Georgia MCGAUGHEY NICHOLLS, Boston, MA (US)

(21) Appl. No.: 19/482,971

(22) PCT Filed: May 10, 2024
(86) PCT No.: PCT/US2024/028693

§ 371 Date: Nov. 10, 2025

Related U.S. Application Data
(60) Provisional application No. 63/501,793, filed on May 12, 2023.

(51) Int. Cl. C12N 9/22; C12N 9/10; C12N 9/78; C12N 15/10; C12N 15/113; C12N 15/52; C12N 15/62; C12N 15/864

(52) U.S. Cl. CPC C12N 9/226; C12N 9/1007; C12N 9/78; C12N 15/102; C12N 15/113; C12N 15/52; C12N 15/625; C12N 15/8645; C12Y 305/04; C12N 2310/20; C12N 2750/14143; C12N 2830/34

(57) ABSTRACT

Disclosed herein are compositions of compact proteins such as compact nucleobase editors that may be used, in some embodiments, for genome editing technologies.

### REFERENCE TO AN ELECTRONIC SEQUENCE LISTING

The contents of the electronic sequence listing (41816_PCT_SequenceListing.xml; Size: 172 KB; and Date of Creation: May 6, 2024) is herein incorporated by reference in its entirety.

### CROSS-REFERENCE TO RELATED APPLICATIONS

This application claims priority to U.S. Provisional Patent Application No. 63/501,793, filed May 12, 2023, the disclosure of which is incorporated by reference herein in its entirety.

### BACKGROUND

Clustered regularly interspaced short palindromic repeats (CRISPR) along with CRISPR associated (Cas) proteins comprise an RNA-guided adaptive immune system in archaea and bacteria. These systems provide immunity by targeting and inactivating nucleic acids that originate from foreign genetic elements. Utilizing technologies such as CRISPR may facilitate correction of genetic disorders due to single base mutations.

Base editors are genome editing technologies that leverage the ability of CRISPR-Cas proteins to find specific sequences of DNA as directed by a modular guide RNA sequence. CRISPR base editor platforms (BE) possess the unique capability to generate precise, user-defined genome-editing events without the need for a donor DNA molecule. BEs are a class of gene editing enzymes that often comprise a Cas nickase fused to a nucleotide deaminase domain. In principle, BEs localize to a target region in the genome guided by a gRNA. Once bound, the Cas9 complexes may displace the nonbound strand, forming a ssDNA R-loop. The R-loop may be rendered accessible to the tethered deaminase domain, whereby cytidine deaminase base editors (CBEs, C:G-to-T:A) may deaminate C-to-U, which base pairs like T, and adenosine deaminase base editors (ABEs, A:T-to-G:C) may deaminate A-to-I, which base pairs like G. Concurrent nicking of the unedited strand by the core Cas9 nickase then may stimulate DNA repair to use the newly deaminated base as a template for DNA polymerization, thereby preserving the edit in both strands of the DNA. In contrast to traditional nuclease-dependent genome editing approaches, BEs do not generate double-stranded DNA breaks, do not require a DNA donor template, and are more efficient in editing nondividing cells, making them attractive agents for in vivo therapeutic genome editing.

Vectors such as AAVs may be used to package genetic information encoding cargo such as BEs. In addition to the base editor itself, it would be desirable for AAVs that encode base editors to also encode the guide RNA, promoters driving base editor and single guide RNA expression, and cis-regulatory elements. Base editors are generally too large to fit into a single AAV, which carry a cargo size limit of about 4.7 kb, not including the inverted terminal repeats (ITRs). Delivery of BEs by AAV has commonly been approached by splitting the BEs between two AAVs and relying on the use of intein trans splicing for the assembly of the full-length effector. Although effective, this approach requires transduction of the target cell by both AAVs and successful in trans splicing of the two intein halves. The requirement to deliver two AAV vectors also increases the viral dosage needed for a treatment, raising safety concerns and adding burdens to AAV manufacturing. There is a need for more compact CRISPR-based cargo (e.g., a BE) that may be encoded by a nucleic acid that can be encapsulated by a single AAV.

### SUMMARY

The present disclosure is directed to truncated proteins (e.g., truncated nucleobase editors) and nucleic acids encoding those truncated proteins.

In a first aspect, the present disclosure is directed to a nucleobase editor comprising a nucleotide deaminase, a Cas9 nuclease, and a linker between the deaminase and Cas9 nuclease, wherein the nucleobase editor is between 1175-1249 amino acids in length. In some embodiments, the nucleobase editor is between 1175-1249, 1180-1249, 1190-1249, 1200-1249, 1210-1249, 1220-1249, 1230-1249, 1240-1249, 1180-1200, 1180-1190, 1200-1249, 1200-1240, 1200-1230, 1200-1220, 1200-1210, 1210-1249, 1210-1240, 1210-1230, 1210-1220, 1220-1249, 1220-1240, 1220-1230, 1230-1249, 1230-1240, or 1240-1249 amino acids in length. In some embodiments, the linker is 2-10, 3-7, 3-5, or 3 amino acids in length. In some embodiments, the nucleotide deaminase is 145-166, 145-162, 145-158, 145-154, 145-151, 150-166, 150-162, 150-156, 150-153, 152-166, 152-158, 152-154, 157-166, 157-162, 161-166, or 161-163 amino acids in length. In some embodiments, the Cas9 nuclease is between 1010-1052, 1010-1040, 1010-1025, 1010-1015, 1020-1052, 1020-1040, 1020-1030, 1030-1052, 1030-1040, 1030-1035, 1035-1046, 1040-1052, 1040-1046, 1045-1052, or 1045-1048 amino acids in length. In some embodiments, the linker comprises one or more proline and one or more alanine. In some embodiments, the nucleotide deaminase comprises an amino acid sequence that is at least 80%, 85%, 90%, 91%, 92%, 93%, 94%, 95%, 96%, 97%, 98%, 99% or 100% identical to the amino acid sequence of SEQ ID NO: 1, or a functional fragment thereof. In some embodiments, the nucleotide deaminase lacks the amino acids corresponding to the first 1, 2, 3, 4, 5, 6, 7, or 8 amino acids of SEQ ID NO: 1. In some embodiments, the nucleotide deaminase lacks the amino acids corresponding to the last 1, 2, 3, 4, 5, 6, 7, 8, 9, or 10 amino acids of SEQ ID NO: 1. In some embodiments, the nucleotide deaminase lacks the amino acids corresponding to the first 6 amino acids of SEQ ID NO: 1 and also lacks the amino acids corresponding to the last 8 amino acids of SEQ ID NO: 1. In some embodiments, the Cas9 nuclease comprises an amino acid sequence that is at least 80%, 85%, 90%, 91%, 92%, 93%, 94%, 95%, 96%, 97%, 98%, 99% or 100% identical to the amino acid sequence of SEQ ID NO: 2, or a functional fragment thereof. In some embodiments, the Cas9 nuclease lacks one or more amino acids from the range of amino acids corresponding to amino acids 1-4, 1-6, 77-85, 124-126, 124-129, 124-143, 124-145, 131-134, 135-138, 122-129, 505-622, 687-688, 716-720, 718-719, 718-730, 765, and/or 865-873 of SEQ ID NO: 2. In some embodiments, the Cas9 nuclease lacks the amino acids corresponding to amino acids 1-4, 1-6, 77-85, 124-126, 124-129, 124-143, 124-145, 131-134, 135-138, 122-129, 505-622, 687-688, 716-720, 718-719, 718-730, 765, and/or 865-873 of SEQ ID NO: 2. In some embodiments, the Cas9 nuclease lacks the amino acids corresponding to amino acids: 718-719 and 765 of SEQ ID NO: 2; 718-719 and 687-688 of SEQ ID NO: 2; 716-720 and 687-688 of SEQ ID NO: 2; 1-4 and 718-730 of SEQ ID NO: 2; 1-4, 687-688, and 716-720 of SEQ ID NO: 2; 1-4 and 124-145 of SEQ ID NO: 2; or 1-4, 505-622, and 718-730 of SEQ ID NO: 2. In some embodiments, the Cas9 nuclease does not lack one or more amino acids from the range of amino acids corresponding to amino acids: 1-8 of SEQ ID NO: 2, 1-10 of SEQ ID NO: 2, 81-94 of SEQ ID NO: 2, 163-164 of SEQ ID NO: 2, 181-184 of SEQ ID NO: 2, 244-424 of SEQ ID NO: 2, 714-769 of SEQ ID NO: 2, 736-742 of SEQ ID NO: 2, 978-979 of SEQ ID NO: 2, and/or 1039-1052 of SEQ ID NO: 2. In some embodiments, the Cas9 nuclease does not lack the amino acid ranges corresponding to amino acids: 1-8 of SEQ ID NO: 2, 1-10 of SEQ ID NO: 2, 81-94 of SEQ ID NO: 2, 163-164 of SEQ ID NO: 2, 181-184 of SEQ ID NO: 2, 244-424 of SEQ ID NO: 2, 714-769 of SEQ ID NO: 2, 736-742 of SEQ ID NO: 2, 978-979 of SEQ ID NO: 2, and/or 1039-1052 of SEQ ID NO: 2. In some embodiments, the nucleobase editor has at least 50%, 60%, 70%, 80%, or 90% base editing activity as compared to a nucleobase editor comprising the sequence of SEQ ID NO: 3. In some embodiments, the base editing activity is assessed using a polynucleotide comprising the nucleotide sequence of SEQ ID NO: 4. In some embodiments, the base editing activity is assessed at position A5, A8 or A13 of SEQ ID NO: 4. In some embodiments, the nucleobase editor further comprises one or more nuclear localization signals (NLSs) and optionally one or more linkers fusing the one or more NLSs to the nucleobase editor. In some embodiments, the one or more NLSs are selected from a c-myc NLS (e.g., an NLS comprising the amino acid sequence of SEQ ID NO: 5), an SV40 NLS (e.g., an NLS comprising the amino acid sequence of SEQ ID NO: 7), and/or a nucleoplasmin NLS (e.g., an NLS comprising the amino acid sequence of SEQ ID NO: 9). In some embodiments, the nucleobase editor comprises: a) a first NLS conjugated to the N-terminus of the nucleobase editor, optionally by means of a first NLS linker, and b) a second NLS conjugated to the C-terminus of the nucleobase editor, optionally by means of a second NLS linker. In some embodiments, the nucleobase editor further comprises: c) a third NLS conjugated to the second NLS, optionally by means of a third NLS linker. In some embodiments, the first NLS comprises the amino acid sequence of SEQ ID NO: 5, wherein the optional first NLS linker comprises the amino acid sequence of SEQ ID NO: 6, wherein the second NLS comprises the amino acid sequence of SEQ ID NO: 7, wherein the optional second NLS linker comprises the amino acid sequence of SEQ ID NO: 8, wherein the third NLS comprises the amino acid sequence of SEQ ID NO: 9, and wherein the optional third NLS linker comprises the amino acid sequence of SEQ ID NO: 10. In some embodiments, the one or more NLSs and one or more linkers fusing the one or more NLSs to the nucleobase editor are collectively no more than 40, 37, 35, 32, 28, or 26 amino acids. In some embodiments, the one or more NLSs and one or more linkers fusing the one or more NLSs to the nucleobase editor are collectively no more than 32 amino acids.

In some embodiments, the amino acid sequence of the linker is SEQ ID NO: 11. In some embodiments, the amino acid sequence of the linker is SEQ ID NO: 12. In some embodiments, the amino acid sequence of the linker is SEQ ID NO: 13. In some embodiments, up to 8 amino acids are deleted from the amino terminus end of the nucleotide deaminase. In some embodiments, up to 8 amino acids are deleted from the carboxy terminus end of the nucleotide deaminase. In some embodiments, up to 8 amino acids are deleted from the carboxy terminus end of the nucleotide deaminase. In some embodiments, up to 22 amino acids are deleted from the REC-lobe domain of the Cas9 nuclease. In some embodiments, the amino acid sequence of the linker is selected from the group of SEQ ID NO: 11, SEQ ID NO: 12, and SEQ ID NO: 13; up to 8 amino acids are deleted from the amino terminus end of the RNA adenine deaminase; up to 8 amino acids are deleted from the carboxy terminus end of the RNA adenine deaminase; and up to 22 amino acids are deleted from the REC-lobe domain of the Cas9 nuclease. In some embodiments, the truncated fusion protein has a total coding size of less than 5 kb. In some embodiments, the truncated fusion protein has a total coding size of less than 4.9 kb. In some embodiments, the truncated fusion protein has a total coding size of less than 4.85 kb.

In some embodiments, the amino acid sequence which encodes the nucleobase editor is at least 80%, 85%, 90%, 91%, 92%, 93%, 94%, 95%, 96%, 97%, 98%, 99% or 100% identical to SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, or SEQ ID NO: 56. In some embodiments, the amino acid sequence which encodes the nucleobase editor is selected from the group SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, and SEQ ID NO: 56.

In some embodiments, the overall activity of the nucleobase editor is at least 30% or greater when compared to the overall activity of wild type nucleobase editor. In some embodiments, the overall activity of the nucleobase editor is at least 50% or greater when compared to the overall activity of wild type nucleobase editor. In some embodiments, the overall activity of the nucleobase editor is at least 75% or greater when compared to the overall activity of wild type nucleobase editor. In some embodiments, the overall activity of the nucleobase editor is at least 80% or greater when compared to the overall activity of wild type nucleobase editor. In some embodiments, the overall activity of the nucleobase editor is at least 85% or greater when compared to the overall activity of wild type nucleobase editor. In some embodiments, the overall activity of the nucleobase editor is at least 90% or greater when compared to the overall activity of wild type nucleobase editor. In some embodiments, the nucleotide deaminase is an RNA adenine deaminase. In some embodiments, the RNA adenine deaminase is a TadA deaminase. In some embodiments, the TadA deaminase is TadA-8e. In some embodiments, the Cas9 nuclease is a Cas9 nickase. In some embodiments, the Cas9 nickase is nSaCas9.

In some embodiments, the nucleobase editor is a truncated nucleobase editor comprising a nucleotide deaminase, a Cas9 nuclease, and a linker between the deaminase and Cas9 nuclease, wherein the nucleobase editor is between 1175-1249 amino acids in length, wherein:
a) the nucleotide deaminase lacks the amino acids corresponding to the first 1, 2, 3, 4, 5, 6, 7, or 8 amino acids of SEQ ID NO: 1 and/or the nucleotide deaminase lacks the amino acids corresponding to the last 1, 2, 3, 4, 5, 6, 7, 8, 9, or 10 amino acids of SEQ ID NO: 1;
b) the Cas9 nuclease lacks one or more amino acids from the range of amino acids corresponding to amino acids 1-4, 1-6, 77-85, 124-126, 124-129, 124-143, 124-145, 131-134, 135-138, 122-129, 505-622, 687-688, 716-720, 718-719, 718-730, 765, and/or 865-873 of SEQ ID NO: 2;
c) the linker between the deaminase and Cas9 nuclease is selected from the group of SEQ ID NO: 11, SEQ ID NO: 12, and SEQ ID NO: 13;
d) the nucleobase editor has at least 50%, 60%, 70%, 80%, or 90% base editing activity as compared to a nucleobase editor comprising the sequence of SEQ ID NO: 3;
e) the nucleobase editor further comprises:
i. one or more nuclear localization signals (NLSs), wherein the one or more NLSs are selected from a c-myc NLS (e.g., an NLS comprising the amino acid sequence of SEQ ID NO: 5), an SV40 NLS (e.g., an NLS comprising the amino acid sequence of SEQ ID NO: 7), and/or a nucleoplasmin NLS (e.g., an NLS comprising the amino acid sequence of SEQ ID NO: 9);
ii. the one or more NLSs are fused to the nucleobase editor optionally through one or more linkers;
iii. wherein the one or more NLSs and one or more linkers fusing the one or more NLSs to the nucleobase editor are collectively no more than 32 amino acids; and
f) the amino acid sequence which encodes the nucleobase editor is selected from the group SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, and SEQ ID NO: 56.

In some embodiments, the truncated nucleobase editor has substantially the same base editing activity as compared to a nucleobase editor comprising the sequence of SEQ ID NO: 3.

Some aspects of the disclosure are directed to a protein comprising a Cas9 protein, wherein the Cas9 protein is between 1010-1052, 1010-1040, 1010-1025, 1010-1015, 1020-1052, 1020-1040, 1020-1030, 1030-1052, 1030-1040, 1030-1035, 1035-1046, 1040-1052, 1040-1046, 1045-1052, or 1045-1048 amino acids in length. In some embodiments, the protein comprises a Cas9 protein, wherein the Cas9 protein comprises an amino acid sequence that is at least 80%, 85%, 90%, 91%, 92%, 93%, 94%, 95%, 96%, 97%, 98%, 99% or 100% identical to the amino acid sequence of SEQ ID NO: 2, or a functional fragment SEQ ID NO: 2. In some embodiments, the Cas9 protein lacks one or more amino acids from the range of amino acids corresponding to amino acids 1-4, 1-6, 77-85, 124-126, 124-129, 124-143, 124-145, 131-134, 135-138, 122-129, 505-622, 687-688, 716-720, 718-719, 718-730, 765, and/or 865-873 of SEQ ID NO: 2. In some embodiments, the Cas9 protein lacks the amino acids corresponding to amino acids 1-4, 1-6, 77-85, 124-126, 124-129, 124-143, 124-145, 131-134, 135-138, 122-129, 505-622, 687-688, 716-720, 718-719, 718-730, 765, and/or 865-873 of SEQ ID NO: 2 (i.e., lacks any one or combination of the foregoing amino acid ranges). In some embodiments, the Cas9 protein lacks the amino acids corresponding to amino acids: 718-719 and 765 of SEQ ID NO: 2; 718-719 and 687-688 of SEQ ID NO: 2; 716-720 and 687-688 of SEQ ID NO: 2; 1-4 and 718-730 of SEQ ID NO: 2; 1-4, 687-688, and 716-720 of SEQ ID NO: 2; 1-4 and 124-145 of SEQ ID NO: 2; or 1-4, 505-622, and 718-730 of SEQ ID NO: 2. In some embodiments, the Cas9 protein does not lack one or more amino acids from the range of amino acids corresponding to amino acids: 1-8 of SEQ ID NO: 2; 1-10 of SEQ ID NO: 2, 81-94 of SEQ ID NO: 2; 163-164 of SEQ ID NO: 2; 181-184 of SEQ ID NO: 2; 244-424 of SEQ ID NO: 2; 714-769 of SEQ ID NO: 2; 736-742 of SEQ ID NO: 2; 978-979 of SEQ ID NO: 2; and/or 1039-1052 of SEQ ID NO: 2. In some embodiments, the Cas9 protein does not lack the amino acid ranges corresponding to amino acids: 1-8 of SEQ ID NO: 2; 1-10 of SEQ ID NO: 2; 81-94 of SEQ ID NO: 2; 163-164 of SEQ ID NO: 2; 181-184 of SEQ ID NO: 2; 244-424 of SEQ ID NO: 2; 714-769 of SEQ ID NO: 2; 736-742 of SEQ ID NO: 2; 978-979 of SEQ ID NO: 2; and/or 1039-1052 of SEQ ID NO: 2. In some embodiments, the Cas9 protein lacks one or more amino acids from the amino acid ranges corresponding to amino acids 81-94, 123-145, 244-424, 714-769, and/or 717-730. In some embodiments, the Cas9 protein lacks the amino acids corresponding to amino acids 81-94, 123-145, 244-424, 714-769, and/or 717-730. In some embodiments, the Cas9 protein is fused to a heterologous moiety. In some embodiments, the heterologous moiety is a deaminase. In some embodiments, the heterologous moiety is a protein useful for gene silencing (e.g., a KRAB domain, Dnmt3A, and/or Dnmt3L protein domains). In some embodiments, the heterologous moiety is a DNA methyltransferase, e.g., Dnmt1, Dnmt3A, or Dnmt3B. In some embodiments, the heterologous moiety is a regulatory factor of a factor of DNA methyltransferase (e.g., a catalytically inactive regulatory factor of DNA methyltransferase (e.g., Dnmt3L)). In some embodiments, the heterologous moiety is a protein useful for reversing gene silencing (e.g., a TET1 DNA demethylase catalytic domain). In some embodiments, the heterologous moiety is a transcriptional repressor. In some embodiments, the heterologous moiety is a transcriptional activator.

Some aspects of the disclosure are directed to a nucleic acid encoding a nucleobase editor comprising a nucleotide deaminase, a Cas9 nuclease, and a linker between the deaminase and Cas9 nuclease, wherein the nucleobase editor is between 1175-1249 amino acids in length. In some embodiments, the nucleic acid further comprises inverted terminal repeats (ITRs). In some embodiments, the nucleic acid further comprises a gRNA (e.g., an sgRNA). In some embodiments, the nucleic acid further comprises promotor sequences. In some embodiments, the entire nucleic acid is less than 5 kb. In some embodiments, the amino acid sequence which encodes the nucleobase editor is at least 80%, 85%, 90%, 91%, 92%, 93%, 94%, 95%, 96%, 97%, 98%, 99% or 100% identical to SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, or SEQ ID NO: 56. In some embodiments, the amino acid sequence which encodes the nucleobase editor is selected from the group SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, and SEQ ID NO: 56.

Some aspects of the disclosure are directed to a vector comprising a nucleic acid encoding a nucleobase editor comprising a nucleotide deaminase, a Cas9 nuclease, and a linker between the deaminase and Cas9 nuclease, wherein the nucleobase editor is between 1175-1249 amino acids in length. In some embodiments, the vector is selected from the group of adeno-associated virus, adenovirus, lentivirus, bacteriophage, and virus-like particles. In some embodiments, the vector is an adeno-associated virus. In some embodiments, the entire nucleic acid is less than 5 kb.

Certain aspects of the current disclosure are directed to an isolated cell comprising a nucleobase editor comprising a nucleotide deaminase, a Cas9 nuclease, and a linker between the deaminase and Cas9 nuclease, wherein the nucleobase editor is between 1175-1249 amino acids in length. In some embodiments, the isolated cell comprises a nucleic acid encoding a nucleobase editor comprising a nucleotide deaminase, a Cas9 nuclease, and a linker between the deaminase and Cas9 nuclease, wherein the nucleobase editor is between 1175-1249 amino acids in length. In some embodiments, the entire nucleic acid is less than 5 kb. In some embodiments, the isolated cell comprises a vector comprising a nucleic acid encoding a nucleobase editor comprising a nucleotide deaminase, a Cas9 nuclease, and a linker between the deaminase and Cas9 nuclease, wherein the nucleobase editor is between 1175-1249 amino acids in length. In some embodiments, the entire nucleic acid is less than 5 kb.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-B shows base editor activity of manipulated base editors as a percentage of wild type base editor activity where the linker between the deaminase and Cas9 nuclease was replaced with either a 3 amino acid linker (SEQ ID NO: 11), a 5 amino acid linker (SEQ ID NO: 12), or a 7 amino acid linker (SEQ ID NO: 13). A) pBE0015 (SEQ ID NO: 14) and pBE0016 (SEQ ID NO: 15) percent activity of wild type nucleobase editor activity as measured at position A5 of SEQ ID NO: 4, the nucleobase editor activity assessment polynucleotide. B) pBE0119 (SEQ ID NO: 16) percent activity of wild type nucleobase editor activity as measured at position A5 of SEQ ID NO: 4.

FIG. 2A-C are graphs showing the base editor activity of manipulated base editors as a percentage of wild type base editor activity where portions of the TadA deaminase were deleted. A) pBE0019 (SEQ ID NO: 17), pBE0020 (SEQ ID NO: 18), and pBE0021 (SEQ ID NO: 19) shown to have over 100% base editor activity to SEQ ID NO: 3 measured at position A8 of SEQ ID NO: 4. B) pBE0057 (SEQ ID NO: 20), pBE0058 (SEQ ID NO: 21), and pBE0059 (SEQ ID NO: 22) percent base editor activity to SEQ ID NO: 3 measured at position A8 of SEQ ID NO: 4. C) pBE0015 (SEQ ID NO: 14) and pBE0117 (SEQ ID NO: 40) percent base editor activity to SEQ ID NO: 3 measured at position A8 of SEQ ID NO: 4.

FIG. 3A-B are graphs where combinations of manipulated base editors were manipulated to remove portions of the TadA deaminase and substitute the linker between the deaminase and Cas9 nuclease with SEQ ID NO: 11, SEQ ID NO: 12, or SEQ ID NO: 13. A) pBE0105 (SEQ ID NO: 34) has 108% base editor activity compared to wild type base editor activity at position A5 of SEQ ID NO: 4. pBE0105 (SEQ ID NO: 34) has the first 4 amino acids of TadA deleted, the last 8 amino acids of TadA deleted, the linker of SEQ ID NO: 12, and the first 4 amino acids of Cas9 nuclease deleted. pBE0107 (SEQ ID NO: 35) shows 184% base editor activity as compared to wild type activity at position A5. pBE0107 (SEQ ID NO: 35) has the first 8 amino acids of TadA deleted, the last 4 amino acids of TadA deleted, the linker of SEQ ID NO: 12, and the first 4 amino acids of Cas9 nuclease deleted. pBE0108 (SEQ ID NO: 36) shows 125% base editor activity as compared to wild type activity at position A5. pBE0108 (SEQ ID NO: 36) has the first 8 amino acids of TadA deleted, the last 6 amino acids of TadA deleted, and the linker of SEQ ID NO: 12. pBE0109 (SEQ ID NO: 37) shows 118% base editor activity as compared to wild type activity at position A5. pBE0109 (SEQ ID NO: 37) has the first 8 amino acids of TadA deleted, the last 6 amino acids of TadA deleted, the linker of SEQ ID NO: 12, and the first 4 amino acids of Cas9 nuclease deleted. B) shows that pBE0110 (SEQ ID NO: 38) has 65% base editor activity as compared to wild type activity at position A8. pBE0110 (SEQ ID NO: 38) has the first 8 amino acids of TadA deleted, the last 8 amino acids of TadA deleted, and the linker of SEQ ID NO: 12.

FIG. 4A-B are graphs showing the base editor activity of manipulated base editors as a percentage of wild type base editor activity where amino acids were deleted from either or both termini of the Cas9 nuclease. A) pBE0042 (SEQ ID NO: 30) has 27% base editor activity compared to wild type base editor activity at position A8 of SEQ ID NO: 4, where pBE0042 (SEQ ID NO: 30) deletes amino acids K2-Y5 from the Cas9 nuclease. pBE043 (SEQ ID NO: 31) has 120% base editor activity compared to wild type base editor activity at position A8 of SEQ ID NO: 4, where pBE0043 (SEQ ID NO: 31) deletes amino acids E1040-G1053 from the carboxy terminus of the Cas9 nuclease. B) pBE0120 (SEQ ID NO: 42) has 112% base editor activity compared to wild type base editor activity at position A8 of SEQ ID NO: 4, where pBE0120 (SEQ ID NO: 42) deletes the first 6 amino acids from the amino terminus of the Cas9 nuclease. pBE0121 (SEQ ID NO: 43) has 13% base editor activity compared to wild type base editor activity at position A8 of SEQ ID NO: 4, where pBE0121 (SEQ ID NO: 43) deletes the first 8 amino acids from the amino terminus of the Cas9 nuclease. pBE0122 (SEQ ID NO: 44) has 0% base editor activity compared to wild type base editor activity at position A8 of SEQ ID NO: 4, where pBE0122 (SEQ ID NO: 44) deletes the first 10 amino acids from the amino terminus of the Cas9 nuclease.

FIG. 5A-C show graphs of the base activity of manipulated base editors as a percentage of wild type base editor activity where amino acids were deleted from the REC Lobe region of the Cas9 nuclease. A) pBE0022 (SEQ ID NO: 23) shows 115% base editor activity compared to wild type base editor activity at position A8 of SEQ ID NO: 4, where pBE0022 (SEQ ID NO: 23) deletes amino acids E125-D127 from the Cas9 nuclease. pBE0024 (SEQ ID NO: 24) shows 82% base editor activity compared to wild type base editor activity at position A8 of SEQ ID NO: 4, where pBE0024 (SEQ ID NO: 24) deletes amino acids E125-N130 from the Cas9 nuclease. pBE0025 (SEQ ID NO: 25) shows 110% base editor activity compared to wild type base editor activity at position A8 of SEQ ID NO: 4, where pBE0025 (SEQ ID NO: 25) deletes amino acids L132-K135 from the Cas9 nuclease. pBE0026 (SEQ ID NO: 26) shows 136% base editor activity compared to wild type base editor activity at position A8 of SEQ ID NO: 4, where pBE0026 (SEQ ID NO: 26) deletes amino acids E136-S139 from the Cas9 nuclease. pBE0027 (SEQ ID NO: 57) shows 5% base editor activity compared to wild type base editor activity at position A8 of SEQ ID NO: 4, where pBE0027 (SEQ ID NO: 57) deletes amino acids V164-R165 from the Cas9 nuclease. B) pBE0028 (SEQ ID NO: 58) shows 1% base editor activity compared to wild type base editor activity at position A8 of SEQ ID NO: 4, where pBE0028 (SEQ ID NO: 58) deletes amino acids Q182-K185 from the Cas9 nuclease. Del3 (SEQ ID NO: 50) shows 99% base editor activity compared to wild type base editor activity at position A8 of SEQ ID NO: 4, where Del3 (SEQ ID NO: 50) deletes amino acids E123-N130 from the Cas9 nuclease. Del4 (SEQ ID NO: 51) shows 25% base editor activity compared to wild type base editor activity at position A8 of SEQ ID NO: 4, where Del4 (SEQ ID NO: 51) deletes amino acids R245-P425 from the Cas9 nuclease. Del5 (SEQ ID NO: 52) shows 53% base editor activity compared to wild type base editor activity at position A8 of SEQ ID NO: 4, where Del 5 (SEQ ID NO: 52) deletes amino acids T78-I86 from the Cas9 nuclease. C) pBE0038 (SEQ ID NO: 28) shows 92% base editor activity compared to wild type base editor activity at position A8 of SEQ ID NO: 4, where pBE0038 (SEQ ID NO: 28) deletes amino acids E125-E146 from the Cas9 nuclease. pBE0039 (SEQ ID NO: 61) shows 0% base editor activity compared to wild type base editor activity at position A8 of SEQ ID NO: 4, where pBE0039 (SEQ ID NO: 61) deletes amino acids E82-G95 from the Cas9 nuclease.

FIG. 6A-C show graphs of the base activity of manipulated base editors as a percentage of wild type base editor activity where amino acids were deleted from the RuvC region of the Cas9 nuclease. A) pBE0029 (SEQ ID NO: 27) shows 96% base editor activity compared to wild type base editor activity at position A8 of SEQ ID NO: 4, where pBE0029 (SEQ ID NO: 27) deletes amino acids K719-Q731 from the Cas9 nuclease. Del2 (SEQ ID NO: 59) shows 3% base editor activity compared to wild type base editor activity at position A8 of SEQ ID NO: 4, where Del2 (SEQ ID NO: 59) deletes amino acids F715-K770 from the Cas9 nuclease. De16 (SEQ ID NO: 53) shows 132% base editor activity compared to wild type base editor activity at position A8 of SEQ ID NO: 4, where De16 (SEQ ID NO: 53) deletes amino acids K689, K719, and K720 from the Cas9 nuclease. B) Del7 (SEQ ID NO: 54) shows 124% base editor activity compared to wild type base editor activity at position A8 of SEQ ID NO: 4, where Del7 (SEQ ID NO: 54) deletes amino acids K719 and K720 from the Cas9 nuclease. Del8 (SEQ ID NO: 55) shows 116% base editor activity compared to wild type base editor activity at position A8 of SEQ ID NO: 4, where Del8 (SEQ ID NO: 55) deletes amino acids W688, K689, K719, and K720 from the Cas9 nuclease. Del9 (SEQ ID NO: 56) shows 102% base editor activity compared to wild type base editor activity at position A8 of SEQ ID NO: 4, where Del9 (SEQ ID NO: 56) deletes amino acids W688, K689, and E717-L721 from the Cas9 nuclease. C) pBE0041 (SEQ ID NO: 62) shows 0% base editor activity compared to wild type base editor activity at position A8 of SEQ ID NO: 4, where pBE0041 (SEQ ID NO: 62) deletes amino acids Q737-E743 from the Cas9 nuclease.

FIG. 7 is a graph of the base activity of a manipulated base editor as a percentage of wild type base editor activity where amino acids were deleted from the PAM-interacting domain of the Cas9 nuclease. Del7 (SEQ ID NO: 54) shows 4% base editor activity compared to wild type base editor activity at position A8 of SEQ ID NO: 4, where Del7 (SEQ ID NO: 54) deletes amino acids Y979 and R980 from the Cas9 nuclease.

FIG. 8 is a graph of the base activity of a manipulated base editor as a percentage of wild type base editor activity where amino acids were deleted from the wedge domain of the Cas9 nuclease. pBE0040 (SEQ ID NO: 29) shows 81% base editor activity compared to wild type base editor activity at position A8 of SEQ ID NO: 4, where pBE0040 (SEQ ID NO: 29) deletes amino acids T866-G847 from the Cas9 nuclease.

FIG. 9A-E show graphs of the base activity of a manipulated base editor as a percentage of wild type base editor activity where combinations of the manipulations were made in the base editors. A) pBE0060 (SEQ ID NO: 32) shows 117% base editor activity compared to wild type base editor activity at position A8 of SEQ ID NO: 4, where pBE0060 (SEQ ID NO: 32) deletes the first and last 4 amino acids from TadA deaminase and has the linker with amino acid sequence of SEQ ID NO: 12. pBE0061 (SEQ ID NO: 33) shows 109% base editor activity compared to wild type base editor activity at position A8 of SEQ ID NO: 4, where pBE0061 (SEQ ID NO: 33) deletes the first and last 4 amino acids from TadA deaminase, has the linker with amino acid sequence of SEQ ID NO: 12, and deletes amino acids E717-L721, W688, and K689 from the Cas9 nuclease. B) pBE0125 (SEQ ID NO: 47) shows 84% base editor activity compared to wild type base editor activity at position A8 of SEQ ID NO: 4 and 121% base editor activity compared to wild type base editor activity at position A5 of SEQ ID NO: 4, where pBE0125 (SEQ ID NO: 47) deletes the first 8 amino acids and the last 6 amino acids from TadA deaminase, has the linker with amino acid sequence of SEQ ID NO: 11, and deletes the first 4 amino acids from the Cas9 nuclease. C) pBE0123 (SEQ ID NO: 45) shows 80% base editor activity compared to wild type base editor activity at position A5 of SEQ ID NO: 4 and 57% base editor activity compared to wild type base editor activity at position A8 of SEQ ID NO: 4, where pBE0123 (SEQ ID NO: 45) deletes the first 8 amino acids and the last 6 amino acids from TadA deaminase, has the linker with amino acid sequence of SEQ ID NO: 12, and deletes the first 4 amino acids as well as amino acids K719-Q731 from the Cas9 nuclease. pBE0124 (SEQ ID NO: 46) shows 48% base editor activity compared to wild type base editor activity at position A5 of SEQ ID NO: 4 and 38% base editor activity compared to wild type base editor activity at position A8 of SEQ ID NO: 4, where pBE0124 (SEQ ID NO: 46) deletes the first 8 amino acids and the last 6 amino acids from TadA deaminase, has the linker with amino acid sequence of SEQ ID NO: 12, and deletes the first 4 amino acids as well as amino acids E717-L721, W688, and K689 from the Cas9 nuclease. D) pBE0126 (SEQ ID NO: 48) shows 84% base editor activity compared to wild type base editor activity at position A5 of SEQ ID NO: 4, where pBE0126 (SEQ ID NO: 48) deletes the first 8 amino acids and the last 6 amino acids from TadA deaminase, has the linker with amino acid sequence of SEQ ID NO: 12, and deletes the first 4 amino acids as well as amino acids E125-E146 from the Cas9 nuclease. E) pBE0127 (SEQ ID NO: 49) shows 38% base editor activity compared to wild type base editor activity at position A5 of SEQ ID NO: 4, where pBE0127 (SEQ ID NO: 49) deletes the first 8 amino acids and the last 6 amino acids from TadA deaminase, has the linker with amino acid sequence of SEQ ID NO: 12, and deletes the first 4 amino acids as well as amino acids E125-E146 and K719-Q731 from the Cas9 nuclease. pBE0129 (SEQ ID NO: 64) shows 0% base editor activity compared to wild type base editor activity at position A5 of SEQ ID NO: 4, where pBE0129 (SEQ ID NO: 64) deletes the first 8 amino acids and the last 6 amino acids from TadA deaminase, has the linker with amino acid sequence of SEQ ID NO: 11, and deletes the first 4 amino acids as well as amino acids E125-E146, E717-L721, and W688-K689 from the Cas9 nuclease.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### DETAILED DESCRIPTION

The following description and examples illustrate embodiments of the present disclosure in detail. It is to be understood that this disclosure is not limited to the particular embodiments described herein and as such can vary. Those of skill in the art will recognize that there are numerous variations and modifications of this disclosure, which are encompassed within its scope.

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Although various features of the present disclosure can be described in the context of a single embodiment, the features can also be provided separately or in any suitable combination. Conversely, although the present disclosure can be described herein in the context of separate embodiments for clarity, the present disclosure can also be implemented in a single embodiment.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only and is not intended to be limiting.

In this application, the use of the singular includes the plural unless specifically stated otherwise. It must be noted that, as used in the specification, the singular forms “a,” “an” and “the” include plural referents unless the context clearly dictates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

Furthermore, use of the term “including” as well as other forms, such as “include”, “includes,” and “included,” is not limiting.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

The term “about” or “approximately” means within an acceptable error range for the particular value as determined by one of ordinary skill in the art, which will depend in part on how the value is measured or determined, e.g., the limitations of the measurement system.

As used herein, the term “edit,” “editing,” or “edited” refers to a method of altering a nucleic acid sequence of a polynucleotide (e.g., for example, a wild type naturally occurring nucleic acid sequence or a mutated naturally occurring sequence) by selective modification (e.g., deletion, insertion, or substitution) of one or more nucleotides in a specific genomic target. Such a specific genomic target includes, but is not limited to, a chromosomal region, a gene, a promoter, an open reading frame or any nucleic acid sequence.

As used herein, the term “target” or “target site” refers to a pre-identified nucleic acid sequence of any composition and/or length. Such target sites include, but are not limited to, a chromosomal region, a gene, a promoter, an open reading frame or any nucleic acid sequence. In some embodiments, the present invention interrogates these specific genomic target sequences with complementary sequences of gRNA.

The term “effective amount” as used herein, refers to a particular amount of a pharmaceutical composition comprising a therapeutic agent that achieves a clinically beneficial result (i.e., for example, a reduction of symptoms). Toxicity and therapeutic efficacy of such compositions can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index, and it can be expressed as the ratio LD50/ED50. Compounds that exhibit large therapeutic indices are preferred. The data obtained from these cell culture assays and additional animal studies can be used in formulating a range of dosage for human use. The dosage of such compounds lies preferably within a range of circulating concentrations that include the ED50 with little or no toxicity. The dosage varies within this range depending upon the dosage form employed, sensitivity of the patient, and the route of administration.

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

The term “viral vector” encompasses any nucleic acid construct derived from a virus genome capable of incorporating heterologous nucleic acid sequences for expression in a host organism. For example, such viral vectors may include, but are not limited to, adeno-associated viral vectors, lentiviral vectors, SV40 viral vectors, retroviral vectors, or adenoviral vectors. Although viral vectors are occasionally created from pathogenic viruses, they may be modified in such a way as to minimize their overall health risk. In some embodiments, this involves the deletion of a part of the viral genome involved with viral replication. Such a virus can efficiently infect cells but, once the infection has taken place, the virus may require a helper virus to provide the missing proteins for production of new virions. Preferably, viral vectors should have a minimal effect on the physiology of the cell it infects and exhibit genetically stable properties (e.g., do not undergo spontaneous genome rearrangement). Most viral vectors are engineered to infect as wide a range of cell types as possible. Even so, a viral receptor can be modified to target the virus to a specific kind of cell. Viruses modified in this manner are said to be pseudotyped. Viral vectors are often engineered to incorporate certain genes that help identify which cells took up the viral genes. These genes are called marker genes. For example, a common marker gene confers antibiotic resistance to a certain antibiotic.

As used herein, the term “Cas9” refers to a protein derived from Type II CRISPR systems. In some embodiments, a Cas9 is an enzyme that is specialized for generating double-strand breaks in DNA, with two active cutting sites (the HNH and RuvC domains), one for each strand of the double helix. In some embodiments, the Cas9 protein lacks the ability to cut both strands of DNA, i.e., it is a nickase. In some embodiments, the Cas9 protein lacks the ability to cut either strand of DNA, i.e., it is “nuclease dead.” In some embodiments, to generate the nickase or nuclease dead Cas9, the Cas9 has one or more deactivating mutations in an HNH nuclease domain (e.g., a mutation corresponding to H840A of SpCas9) and/or a deactivating mutation in a RuvC nuclease domain (e.g., a mutation corresponding to D10A of SpCas9)). In some embodiments, the Cas9 comprises a D at the amino acid position corresponding to position 9 of SEQ ID NO: 2. In some embodiments, the Cas9 comprises a D at the amino acid position corresponding to position 9 of SEQ ID NO: 65. In some embodiments, the Cas9 comprises an amino acid other than D (e.g., A) at the amino acid position corresponding to position 9 of SEQ ID NO: 2. In some embodiments, the Cas9 comprises an amino acid other than D (e.g., A) at the amino acid position corresponding to position 9 of SEQ ID NO: 65. In some embodiments, the Cas9 comprises an N at the amino acid position corresponding to position 579 of SEQ ID NO: 2. In some embodiments, the Cas9 comprises an N at the amino acid position corresponding to position 581 of SEQ ID NO: 65. In some embodiments, the Cas9 comprises an amino acid other than N (e.g., A) at the amino acid position corresponding to position 579 of SEQ ID NO: 2. In some embodiments, the Cas9 comprises an amino acid other than N (e.g., A) at the amino acid position corresponding to position 581 of SEQ ID NO: 65. In preferred embodiments, any of the Cas9 proteins disclosed herein is capable of binding to DNA (e.g., when complexed with a sgRNA). In some embodiments, tracrRNA and spacer RNA have been combined into a “single-guide RNA” (sgRNA) molecule that, mixed with Cas9, could find and cleave DNA targets through Watson-Crick pairing between the guide sequence within the sgRNA and the target DNA sequence. Jinek et al., “A programmable dual-RNA-guided DNA endonuclease in adaptive bacterial immunity” Science 337(6096):816-821 (2012). In some embodiments, the Cas9 protein may be fused to a heterologous moiety. (e.g., TadA). In some embodiments, the heterologous moiety is a protein useful for gene silencing (e.g., a KRAB domain, Dnmt3A, and/or Dnmt3L protein domains). In some embodiments, the heterologous moiety is a DNA methyltransferase, e.g., Dnmt1, Dnmt3A, or Dnmt3B. In some embodiments, the heterologous moiety is a regulatory factor of a factor of DNA methyltransferase (e.g., a catalytically inactive regulatory factor of DNA methyltransferase (e.g., Dnmt3L)). In some embodiments, the heterologous moiety is a protein useful for reversing gene silencing (e.g., a TET1 DNA demethylase catalytic domain) In some embodiments, the heterologous moiety is a transcriptional repressor. In some embodiments, the heterologous moiety is a transcriptional activator.

The term “KRAB domain” or “Kruppel associated box domain” as used herein refers to a category of transcriptional repression domains that are usually 45-75 amino acids in length. See, e.g., Ecco, G., Imbeault, M., Trono, D., KRAB zinc finger proteins, Development 144, 2017; Lambert et al. The human transcription factors, Cell 172, 2018. In some embodiments, the KRAB domain is a KRAB domain of Kox 1. In some embodiments, the KRAB domain comprises the sequence of SEQ ID NO: 73. In some embodiments, the KRAB domain comprises an amino acid sequence that is at least 50%, 55%, 60%, 65%, 70%, 75%, 80%, 85%, 90%, 91%, 92%, 93%, 94%, 95%, 96%, 97%, 98%, 99% or 100% identical to SEQ ID NO: 73:

DAKSLTAWSRTLVTFKDVFVDFTREEWKLLDTAQQIVYRNVMLENYKNLV
SLGYQLTKPDVILRLEKGEEP.

The term “Dnmt3A” as used herein refers to a “DNA (cytosine-5)-methyltransferase 3A” or “DNA methyltransferase 3a” protein or fragment thereof. In some embodiments, the Dnmt3A comprises the sequence of SEQ ID NO: 74. In some embodiments, the Dnmt3A comprises an amino acid sequence that is at least 50%, 55%, 60%, 65%, 70%, 75%, 80%, 85%, 90%, 91%, 92%, 93%, 94%, 95%, 96%, 97%, 98%, 99% or 100% identical to SEQ ID NO: 74:

NHDQEFDPPKVYPPVPAEKRKPIRVLSLFDGIATGLLVLKDLGIQVDRYI
ASEVCEDSITVGMVRHQGKIMYVGDVRSVTQKHIQEWGPFDLVIGGSPCN
DLSIVNPARKGLYEGTGRLFFEFYRLLHDARPKEGDDRPFFWLFENVVAM
GVSDKRDISRFLESNPVMIDAKEVSAAHRARYFWGNLPGMNRPLASTVND
KLELQECLEHGRIAKFSKVRTITTRSNSIKQGKDQHFPVFMNEKEDILWC
TEMERVFGFPVHYTDVSNMSRLARQRLLGRSWSVPVIRHLFAPLKEYFAC
V.

The term “Dnmt3L” as used herein refers to a “DNA (cytosine-5)-methyltransferase 3L” or “DNA methyltransferase 3L” protein or fragment thereof. In some embodiments, the Dnmt3L comprises the sequence of SEQ ID NO: 75. In some embodiments, the Dnmt3L comprises an amino acid sequence that is at least 50%, 55%, 60%, 65%, 70%, 75%, 80%, 85%, 90%, 91%, 92%, 93%, 94%, 95%, 96%, 97%, 98%, 99% or 100% identical to SEQ ID NO: 75:

MGPMEIYKTVSAWKRQPVRVLSLFRNIDKVLKSLGFLESGSGSGGGTLKY
VEDVTNVVRRDVEKWGPFDLVYGSTQPLGSSCDRCPGWYMFQFHRILQYA
LPRQESQRPFFWIFMDNLLLTEDDQETTTRFLQTEAVTLQDVRGRDYQNA
MRVWSNIPGLKSKHAPLTPKEEEYLQAQVRSRSKLDAPKVDLLVKNCLLP
LREYFKYFSQNSLPL.

The term “protospacer adjacent motif” (or PAM) as used herein, refers to a DNA sequence that may be required for a Cas9/sgRNA to form an R-loop to interrogate a specific DNA sequence through Watson-Crick pairing of its guide RNA with the genome. The PAM specificity may be a function of the DNA-binding specificity of the Cas9 protein (e.g., a “protospacer adjacent motif recognition domain” at the C-terminus of Cas9).

“Guide RNA”, “gRNA”, and simply “guide”, are used herein interchangeably to refer to either a crRNA (also known as CRISPR RNA), or the combination of a crRNA and a trRNA (also known as tracrRNA). The crRNA and trRNA may be associated as a single RNA molecule (single guide RNA, sgRNA) or in two separate RNA molecules (dual guide RNA, dgRNA). “Guide RNA” refers to each type. The trRNA may be a naturally occurring sequence, or a trRNA sequence with modifications or variations compared to naturally occurring sequences. For clarity, the terms “guide RNA” or “guide” as used herein, and unless specifically stated otherwise, may refer to an RNA molecule (comprising A, C, G, and U nucleotides) or to a DNA molecule encoding such an RNA molecule (comprising A, C, G, and T nucleotides) or complementary sequences thereof. In general, in the case of a DNA nucleic acid construct encoding a guide RNA, the U residues in any of the RNA sequences described herein may be replaced with T residues, and in the case of a guide RNA construct encoded by any of the DNA sequences described herein, the T residues may be replaced with U residues.

As used herein, the term “sgRNA” refers to single guide RNA used in conjunction with CRISPR associated systems (Cas). sgRNAs are a fusion of crRNA and tracrRNA and contain nucleotides of sequence complementary to the desired target site. Watson-Crick pairing of the sgRNA with the target site permits R-loop formation, which in conjunction with a functional PAM permits DNA cleavage or in the case of nuclease-deficient Cas9 allows binds to the DNA at that locus.

As used herein, a “spacer sequence,” sometimes also referred to herein and in the literature as a “spacer,” “protospacer,” “guide sequence,” or “targeting sequence” refers to a sequence within a guide RNA that is complementary to a target sequence and functions to direct a guide RNA to a target sequence for cleavage by a Cas9. For clarity, the terms “spacer sequence”, “spacer,” “protospacer,” “guide sequence,” or “targeting sequence” as used herein, and unless specifically stated otherwise, may refer to an RNA molecule (comprising A, C, G, and U nucleotides) or to a DNA molecule encoding such an RNA molecule (comprising A, C, G, and T nucleotides) or complementary sequences thereof. A guide sequence can be 24, 23, 22, 21, 20 or fewer base pairs in length, e.g., in the case of Staphylococcus lugdunensis (i.e., SluCas9) or Staphylococcus aureus (i.e., SaCas9) and related Cas9 homologs/orthologs. In preferred embodiments, a guide/spacer sequence in the case of SluCas9 or SaCas9 is at least 20 base pairs in length, or more specifically, within 20-25 base pairs in length (see, e.g., Schmidt et al., 2021, Nature Communications, “Improved CRISPR genome editing using small highly active and specific engineered RNA-guided nucleases”). Shorter or longer sequences can also be used as guides, e.g., 15-, 16-, 17-, 18-, 19-, 20-, 21-, 22-, 23-, 24-, or 25-nucleotides in length.

As used herein, the term “fluorescent protein” refers to a protein domain that comprises at least one organic compound moiety that emits fluorescent light in response to the appropriate wavelengths. For example, fluorescent proteins may emit red, blue and/or green light. Such proteins are readily commercially available including, but not limited to: i) mCherry (Clonetech Laboratories): excitation: 556/20 nm (wavelength/bandwidth); emission: 630/91 nm; ii) sfGFP (Invitrogen): excitation: 470/28 nm; emission: 512/23 nm; iii) TagBFP (Evrogen): excitation 387/11 nm; emission 464/23 nm.

As used herein, the term “orthogonal” refers to targets that are non-overlapping, uncorrelated, or independent. For example, if two orthogonal nuclease-deficient Cas9 gene fused to different effector domains were implemented, the sgRNAs coded for each would not cross-talk or overlap. Not all nuclease-deficient Cas9 genes operate the same, which enables the use of orthogonal nuclease-deficient Cas9 gene fused to a different effector domains provided the appropriate orthogonal sgRNAs.

As used herein, the term “phenotypic change” or “phenotype” refers to the composite of an organism's observable characteristics or traits, such as its morphology, development, biochemical or physiological properties, phenology, behavior, and products of behavior.

Phenotypes result from the expression of an organism's genes as well as the influence of environmental factors and the interactions between the two.

“Nucleic acid sequence”, “polynucleotide sequence”, and “nucleotide sequence” as used herein refer to an oligonucleotide or polynucleotide, and fragments or portions thereof, and to DNA or RNA of genomic or synthetic origin which may be single- or double-stranded, and represent the sense or antisense strand.

The term “an isolated nucleic acid”, as used herein, refers to any nucleic acid molecule that has been removed from its natural state (e.g., removed from a cell and is, in a preferred embodiment, free of other genomic nucleic acid).

The terms “amino acid sequence” and “polypeptide sequence” as used herein, are interchangeable and refer to a sequence of amino acids.

As used herein the term “portion” when in reference to a protein (as in “a portion of a given protein”) refers to fragments of that protein. Unless specified otherwise, the fragments may range in size from four amino acid residues to the entire amino acid sequence minus one amino acid.

The term “portion” when used in reference to a nucleotide sequence refers to fragments of that nucleotide sequence. Unless specified otherwise, the fragments may range in size from 5 nucleotide residues to the entire nucleotide sequence minus one nucleic acid residue.

As used herein, the terms “complementary” or “complementarity” are used in reference to “polynucleotides” and “oligonucleotides” (which are interchangeable terms that refer to a sequence of nucleotides) related by the base-pairing rules. For example, the sequence “C-A-G-T,” is complementary to the sequence “G-T-C-A.” Complementarity can be “partial” or “total.” “Partial” complementarity is where one or more nucleic acid bases is not matched according to the base pairing rules. “Total” or “complete” complementarity between nucleic acids is where each and every nucleic acid base is matched with another base under the base pairing rules. The degree of complementarity between nucleic acid strands has significant effects on the efficiency and strength of hybridization between nucleic acid strands. This is of particular importance in amplification reactions, as well as detection methods which depend upon binding between nucleic acids.

The terms “homology” and “homologous” as used herein in reference to nucleotide sequences refer to a degree of complementarity with other nucleotide sequences. There may be partial homology or complete homology (i.e., identity). A nucleotide sequence which is partially complementary, i.e., “substantially homologous,” to a nucleic acid sequence is one that at least partially inhibits a completely complementary sequence from hybridizing to a target nucleic acid sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous sequence to a target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target sequence which lacks even a partial degree of complementarity (e.g., less than about 30% identity); in the absence of non-specific binding the probe will not hybridize to the second non-complementary target.

The terms “homology” and “homologous” as used herein in reference to amino acid sequences refer to the degree of identity of the primary structure between two amino acid sequences. Such a degree of identity may be directed to a portion of each amino acid sequence, or to the entire length of the amino acid sequence. Two or more amino acid sequences that are “substantially homologous” may have at least 75% identity, at least 85% identity, at least 95%, or 100% identity.

An oligonucleotide sequence which is a “homolog” is defined herein as an oligonucleotide sequence which exhibits greater than or equal to 50% identity to a sequence, when sequences having a length of 100 bp or larger are compared.

As used herein, the term “hybridization” is used in reference to the pairing of complementary nucleic acids using any process by which a strand of nucleic acid joins with a complementary strand through base pairing to form a hybridization complex. Hybridization and the strength of hybridization (i.e., the strength of the association between the nucleic acids) is impacted by such factors as the degree of complementarity between the nucleic acids, stringency of the conditions involved, the melting temperature of the formed hybrid, and the G:C ratio within the nucleic acids.

As used herein the term “hybridization complex” refers to a complex formed between two nucleic acid sequences by virtue of the formation of hydrogen bounds between complementary G and C bases and between complementary A and T (or U) bases; these hydrogen bonds may be further stabilized by base stacking interactions. The two complementary nucleic acid sequences hydrogen bond in an antiparallel configuration. A hybridization complex may be formed in solution (e.g., Co t or Ro t analysis) or between one nucleic acid sequence present in solution and another nucleic acid sequence immobilized to a solid support (e.g., a nylon membrane or a nitrocellulose filter as employed in Southern and Northern blotting, dot blotting or a glass slide as employed in in situ hybridization, including FISH (fluorescent in situ hybridization)).

DNA and RNA molecules are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. As used herein, a nucleic acid sequence, even if internal to a larger oligonucleotide, also may be said to have 5′ and 3′ ends. In either a linear or circular DNA or RNA molecule, discrete elements are referred to as being upstream” or 5′ of the “downstream” or 3′ elements. This terminology reflects the fact that transcription proceeds in a 5′ to 3′ fashion along the DNA or RNA strand. The promoter and enhancer elements which direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

The term “transfection” or “transfected” refers to the introduction of foreign DNA or RNA into a cell.

As used herein, the terms “nucleic acid molecule encoding”, “DNA (or RNA) sequence encoding,” and “DNA (or RNA) encoding” refer to the order or sequence of nucleotides along a strand of nucleic acid. The order of these deoxyribonucleotides determines the order of amino acids along the polypeptide (protein) chain. The DNA sequence thus codes for the amino acid sequence.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences which direct the termination of transcription, posttranscriptional cleavage and polyadenylation.

The terms “patient,” “subject,” and “individual” may be used interchangeably and refer to either a human or a non-human animal. The “non-human animals” and “non-human mammals” as used interchangeably herein, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g., dog, cat, horse, and the like, or production mammal, e.g. cow, sheep, pig, and the like. “Patient in need thereof” or “subject in need thereof” is referred to herein as a patient diagnosed with or suspected of having a disease or disorder, for instance, but not restricted to diabetes.

“Administering” as used herein can refer to providing one or more compositions described herein to a patient or a subject. By way of example and not limitation, composition administration, e.g., injection, can be performed by intravenous (i.v.) injection, sub-cutaneous (s.c.) injection, intradermal (i.d.) injection, intraperitoneal (i.p.) injection, or intramuscular (i.m.) injection. One or more such routes can be employed. Parenteral administration can be, for example, by bolus injection or by gradual perfusion over time. Alternatively, or concurrently, administration can be by the oral route. Additionally, administration can also be by surgical deposition of a bolus or pellet of cells, or positioning of a medical device. In an embodiment, a composition of the present disclosure can comprise engineered cells or host cells expressing nucleic acid sequences described herein, or a vector comprising at least one nucleic acid sequence described herein, in an amount that is effective to treat or prevent proliferative disorders. A pharmaceutical composition can comprise the cell population as described herein, in combination with one or more pharmaceutically or physiologically acceptable carriers, diluents or excipients. Such compositions can comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

The term “genetically engineered” and its grammatical equivalents as used herein refer to a non-naturally occurring genetic modification. Examples of genetic engineering include use of gene editing systems such as the CRISPR/Cas, the TALEN, and/or zinc finger systems for disrupting the expression (e.g., to reduce or eliminate expression) of one or more gene targets in a cell, or for increasing the expression (e.g., by inserting a gene of interest) into a cell. A “genetically engineered” cell, as used herein, means a cell that was genetically engineered, or a cell that was derived and/or descended from a cell that was genetically engineered.

The disclosure contemplates truncated proteins lacking one or more amino acids. In some embodiments, the truncated proteins lack 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 67, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129 or 130 amino acids (e.g., contiguous amino acids) as compared to a reference sequence (e.g., SEQ ID NO: 2), or to a recited portion of a reference sequence (e.g., the truncated protein lacks 100 of the amino acids corresponding to amino acids 505-622 of SEQ ID NO: 2). In some embodiments, the truncated protein lacks 1-130, 1-120, 1-110, 1-100, 1-90, 1-80, 1-70, 1-60, 1-50, 1-40, 1-30, 1-20, 1-15, 1-10, 1-5, 1-3, 100-130, 100-110, 90-110, 80-100, 60-80, 40-60, 20-40, 10-70, 10-50, 10-30, 10-20, 10-15, 5-10, 5-20, or 20-30 amino acids as compared to a reference sequence (e.g., SEQ ID NO: 2), or to a recited portion of a reference sequence (e.g., the truncated protein lacks 90-110 of the amino acids corresponding to amino acids 505-622 of SEQ ID NO: 2). In some embodiments, the truncated protein lacks contiguous amino acids of any of the sequences disclosed herein. In some embodiments, the truncated protein lacks non-contiguous amino acids of any of the sequences disclosed herein.

### Compact Protein Composition

In a first aspect, the present disclosure is directed to a compact protein comprising a nucleotide deaminase, a Cas9 nuclease, and a linker between the deaminase and Cas9 nuclease, wherein the compact protein is between 1175-1249 amino acids in length. In some embodiments and the compact protein is between 1175-1249 amino acids in length. In some embodiments, the compact protein is between 1180-1249 amino acids in length. In some embodiments, the compact protein is between 1190-1249 amino acids in length. In some embodiments, the compact protein is between 1200-1249 amino acids in length. In some embodiments, the compact protein is between 1210-1249 amino acids in length. In some embodiments, the compact protein is between 1220-1249 amino acids in length. In some embodiments, the compact protein is between 1230-1249 amino acids in length. In some embodiments, the compact protein is between 1240-1249 amino acids in length. In some embodiments, the compact protein is between 1180-1200 amino acids in length. In some embodiments, the compact protein is between 1180-1190 amino acids in length. In some embodiments, the compact protein is between 1200-1249 amino acids in length. In some embodiments, the compact protein is between 1200-1240 amino acids in length. In some embodiments, the compact protein is between 1200-1230 amino acids in length. In some embodiments, the compact protein is between 1200-1220 amino acids in length. In some embodiments, the compact protein is between 1200-1210 amino acids in length. In some embodiments, the compact protein is between 1210-1249 amino acids in length. In some embodiments, the compact protein is between 1210-1240 amino acids in length. In some embodiments, the compact protein is between 1210-1230 amino acids in length. In some embodiments, the compact protein is between 1210-1220 amino acids in length. In some embodiments, the compact protein is between 1220-1249 amino acids in length. In some embodiments, the compact protein is between 1220-1240 amino acids in length. In some embodiments, the compact protein is between 1220-1230 amino acids in length. In some embodiments, the compact protein is between 1230-1249 amino acids in length. In some embodiments, the compact protein is between 1230-1240 amino acids in length. In some embodiments, the compact protein is between 1240-1249 amino acids in length.

In certain aspects of the present disclosure, the compact protein is a nucleobase editor comprising a nucleotide deaminase, a Cas9 nuclease, and a linker between the deaminase and Cas9 nuclease. In some embodiments, the nucleobase editor is between 1175-1249 amino acids in length. In some embodiments and the nucleobase editor is between 1175-1249 amino acids in length. In some embodiments, the nucleobase editor is between 1180-1249 amino acids in length. In some embodiments, the nucleobase editor is between 1190-1249 amino acids in length. In some embodiments, the nucleobase editor is between 1200-1249 amino acids in length. In some embodiments, the nucleobase editor is between 1210-1249 amino acids in length. In some embodiments, the nucleobase editor is between 1220-1249 amino acids in length. In some embodiments, the nucleobase editor is between 1230-1249 amino acids in length. In some embodiments, the nucleobase editor is between 1240-1249 amino acids in length. In some embodiments, the nucleobase editor is between 1180-1200 amino acids in length. In some embodiments, the nucleobase editor is between 1180-1190 amino acids in length. In some embodiments, the nucleobase editor is between 1200-1249 amino acids in length. In some embodiments, the nucleobase editor is between 1200-1240 amino acids in length. In some embodiments, the nucleobase editor is between 1200-1230 amino acids in length. In some embodiments, the nucleobase editor is between 1200-1220 amino acids in length. In some embodiments, the nucleobase editor is between 1200-1210 amino acids in length. In some embodiments, the nucleobase editor is between 1210-1249 amino acids in length. In some embodiments, the nucleobase editor is between 1210-1240 amino acids in length. In some embodiments, the nucleobase editor is between 1210-1230 amino acids in length. In some embodiments, the nucleobase editor is between 1210-1220 amino acids in length. In some embodiments, the nucleobase editor is between 1220-1249 amino acids in length. In some embodiments, the nucleobase editor is between 1220-1240 amino acids in length. In some embodiments, the nucleobase editor is between 1220-1230 amino acids in length. In some embodiments, the nucleobase editor is between 1230-1249 amino acids in length. In some embodiments, the nucleobase editor is between 1230-1240 amino acids in length. In some embodiments, the nucleobase editor is between 1240-1249 amino acids in length.

### Nucleotide Deaminase

In some embodiments, the nucleotide deaminase is 145-166 amino acids in length. In some embodiments, the nucleotide deaminase is 145-162 amino acids in length. In some embodiments, the nucleotide deaminase is 145-158 amino acids in length. In some embodiments, the nucleotide deaminase is 145-154 amino acids in length. In some embodiments, the nucleotide deaminase is 145-151 amino acids in length. In some embodiments, the nucleotide deaminase is 150-166 amino acids in length. In some embodiments, the nucleotide deaminase is 150-162 amino acids in length. In some embodiments, the nucleotide deaminase is 150-156 amino acids in length. In some embodiments, the nucleotide deaminase is 150-153 amino acids in length. In some embodiments, the nucleotide deaminase is 152-166 amino acids in length. In some embodiments, the nucleotide deaminase is 152-158 amino acids in length. In some embodiments, the nucleotide deaminase is 152-154 amino acids in length. In some embodiments, the nucleotide deaminase is 157-166 amino acids in length. In some embodiments, the nucleotide deaminase is 157-162 amino acids in length. In some embodiments, the nucleotide deaminase is 161-166 amino acids in length. In some embodiments, the nucleotide deaminase is 161-163 amino acids in length.

In some embodiments, any of the truncated nucleotide deaminases disclosed herein is capable of deaminating adenine. In some embodiments, any of the nucleotide deaminases is capable of deaminating cytosine. In some embodiments, the truncated deaminase protein is capable of deaminating adenine at least 10%, 20%, 30%, 40%, 50%, 60%, 70%, 80%, 90%, or 100% as efficiently as a reference untruncated deaminase protein (e.g., a deaminase protein comprising the amino acid sequence of SEQ ID NO: 1) under the same conditions. In some embodiments, the truncated deaminase protein is capable of deaminating adenine 10-100%, 10-80%, 10-60%, 10-40%, 10-20%, 30-100%, 30-80%, 30-60%, 30-40%, 50-100%, 50-80%, 50-60%, 70-100%, 70-80%, 80-90%, 90-100%, or 85-95% as efficiently as a reference untruncated deaminase protein (e.g., a deaminase protein comprising the amino acid sequence of SEQ ID NO: 1) for the same target sequence under the same conditions. In some embodiments, the truncated deaminase protein is capable of deaminating cytosine at least 10%, 20%, 30%, 40%, 50%, 60%, 70%, 80%, 90%, or 100% as efficiently as a reference untruncated deaminase protein (e.g., a deaminase protein comprising the amino acid sequence of SEQ ID NO: 1) under the same conditions. In some embodiments, the truncated deaminase protein is capable of deaminating cytosine 10-100%, 10-80%, 10-60%, 10-40%, 10-20%, 30-100%, 30-80%, 30-60%, 30-40%, 50-100%, 50-80%, 50-60%, 70-100%, 70-80%, 80-90%, 90-100%, or 85-95% as efficiently as a reference untruncated deaminase protein (e.g., a deaminase protein comprising the amino acid sequence of SEQ ID NO: 1) for the same target sequence under the same conditions.

In some embodiments, the nucleotide deaminase is an adenine deaminase. In some embodiments, the adenine deaminase is a TadA deaminase. In some embodiments, the deaminase is APOBECI, or any of the APOBECI deaminases disclosed in US20170121693, which is incorporated by reference herein in its entirety. In some embodiments, the TadA deaminase is TadA-8e. In some embodiments, the nucleotide deaminase comprises an amino acid sequence that is at least 80%, 85%, 90%, 91%, 92%, 93%, 94%, 95%, 96%, 97%, 98%, 99% or 100% identical to the amino acid sequence of SEQ ID NO: 1, or a functional fragment of SEQ ID NO: 1. In some embodiments, the nucleotide deaminase lacks the amino acids corresponding to the first amino acid of SEQ ID NO: 1. In some embodiments, the nucleotide deaminase lacks the amino acids corresponding to the first 2 amino acids of SEQ ID NO: 1. In some embodiments, the nucleotide deaminase lacks the amino acids corresponding to the first 3 amino acids of SEQ ID NO: 1. In some embodiments, the nucleotide deaminase lacks the amino acids corresponding to the first 4 amino acids of SEQ ID NO: 1. In some embodiments, the nucleotide deaminase lacks the amino acids corresponding to the first 5 amino acids of SEQ ID NO: 1. In some embodiments, the nucleotide deaminase lacks the amino acids corresponding to the first 6 amino acids of SEQ ID NO: 1. In some embodiments, the nucleotide deaminase lacks the amino acids corresponding to the first 7 amino acids of SEQ ID NO: 1. In some embodiments, the nucleotide deaminase lacks the amino acids corresponding to the first 8 amino acids of SEQ ID NO: 1. In some embodiments, the nucleotide deaminase lacks the amino acids corresponding to the last 1, 2, 3, 4, 5, 6, 7, 8, 9, or 10 amino acids of SEQ ID NO: 1. In some embodiments, the nucleotide deaminase lacks the amino acids corresponding to the first 6 amino acids of SEQ ID NO: 1 and also lacks the amino acids corresponding to the last 8 amino acids of SEQ ID NO: 1:

SEVEFSHEYWMRHALTLAKRARDEREVPVGAVLVLNNRVIGEGWNRAIGL
HDPTAHAEIMALRQGGLVMQNYRLIDATLYVTFEPCVMCAGAMIHSRIGR
VVFGVRNSKRGAAGSLMNVLNYPGMNHRVEITEGILADECAALLCDFYRM
PRQVFNAQKKAQSSIN

In some embodiments, the nucleotide deaminase is a cytosine deaminase. In some embodiments, the nucleotide deaminase is any of the modified deaminases disclosed in Chen et al., 2022, Nature Biotechnology, 41:663-672. In some embodiments, the nucleotide deaminase comprises an amino acid sequence that is at least 80%, 85%, 90%, 91%, 92%, 93%, 94%, 95%, 96%, 97%, 98%, 99% or 100% identical to the amino acid sequence of SEQ ID NO: 1, or a functional fragment of SEQ ID NO: 1, but wherein the amino acid corresponding to amino acid position 45 of SEQ ID NO: 1 is not N. In some embodiments, the nucleotide deaminase comprises an amino acid sequence that is at least 80%, 85%, 90%, 91%, 92%, 93%, 94%, 95%, 96%, 97%, 98%, 99% or 100% identical to the amino acid sequence of SEQ ID NO: 1, or a functional fragment of SEQ ID NO: 1, but wherein the amino acid corresponding to amino acid position 45 of SEQ ID NO: 1 is L.

### Linker Between the Deaminase and the Nuclease

There is a linker between the deaminase and the nuclease. This linker connects the deaminase and the nuclease of the nucleobase editor described herein. In some embodiments, the linker between the deaminase and the nuclease is 2-10 amino acids in length. In some embodiments, the linker between the deaminase and the nuclease is 3-7 amino acids in length. In some embodiments, the linker between the deaminase and the nuclease is 3-5 amino acids in length. In some embodiments, the linker between the deaminase and the nuclease is 3 amino acids in length. In some embodiments, the linker between the deaminase and the nuclease comprises one or more proline and one or more alanine. In some embodiments, the amino acid sequence of the linker between the deaminase and the nuclease is SEQ ID NO: 11: PAP. In some embodiments, the amino acid sequence of the linker between the deaminase and the nuclease is SEQ ID NO: 12: PAPAP. In some embodiments, the amino acid sequence of the linker between the deaminase and the nuclease is SEQ ID NO: 13: PAPAPAP. In some embodiments, the linker comprises glycine. In some embodiments, the linker comprises 1, 2, 3, 4, 5, 6, 7, 8, or 9 glycine residues. In some embodiments, the linker comprises serine. In some embodiments, the linker comprises 1, 2, 3, 4, 5, 6, 7, 8, or 9 serine residues. In some embodiments, the linker comprises one or more proline residues. In some embodiments, the linker comprises one or more alanine residues. In some embodiments, the linker comprises one or more threonine residues. In some embodiments, the linker comprises an amino acid sequence that is at least 80%, 85%, 90%, 91%, 92%, 93%, 94%, 95%, 96%, 97%, 98%, 99%, or 100% identical to the amino acid sequence of SEQ ID NO: 78 (SGGSSGGSSGSETPGTSESATPESSGGSSGGS). In some embodiments, the linker comprises the amino acid sequence of SEQ ID NO: 79 (GGSS), SEQ ID NO: 80 (GGSSGG) or SEQ ID NO: 81 (SGGSSGGS).

### Cas Nuclease

In some embodiments, the disclosure provides for truncated Cas proteins, e.g., truncated Cas9 proteins, as compared to a reference Cas protein sequence (e.g., a wildtype SaCas9 or SluCas9 protein). In some embodiments, the Cas9 nuclease of the nucleobase editor is between 1010-1052 amino acids in length. In some embodiments, the Cas9 nuclease is between 1010-1040 amino acids in length. In some embodiments, the Cas9 nuclease is between 1010-1025 amino acids in length. In some embodiments, the Cas9 nuclease is between 1010-1015 amino acids in length. In some embodiments, the Cas9 nuclease is between 1020-1052 amino acids in length. In some embodiments, the Cas9 nuclease is between 1020-1040 amino acids in length. In some embodiments, the Cas9 nuclease is between 1020-1030 amino acids in length. In some embodiments, the Cas9 nuclease is between 1030-1052 amino acids in length. In some embodiments, the Cas9 nuclease is between 1030-1040 amino acids in length. In some embodiments, the Cas9 nuclease is between 1030-1035 amino acids in length. In some embodiments, the Cas9 nuclease is between 1035-1046 amino acids in length. In some embodiments, the Cas9 nuclease is between 1040-1052 amino acids in length. In some embodiments, the Cas9 nuclease is between 1040-1046 amino acids in length. In some embodiments, the Cas9 nuclease is between 1045-1052 amino acids in length. In some embodiments, the Cas9 nuclease is between 1045-1048 amino acids in length.

In some embodiments, the Cas (e.g., Cas9) nuclease is a Cas (e.g., Cas9) nickase. In some embodiments, the Cas9 nickase is nSaCas9. In some embodiments, the Cas9 nuclease comprises an amino acid sequence that is at least 80%, 85%, 90%, 91%, 92%, 93%, 94%, 95%, 96%, 97%, 98%, 99% or 100% identical to the amino acid sequence of SEQ ID NO: 2, or a functional fragment of SEQ ID NO: 2:

KRNYILGLAIGITSVGYGIIDYETRDVIDAGVRLFKEANVENNEGRRSKR
GARRLKRRRRHRIQRVKKLLFDYNLLTDHSELSGINPYEARVKGLSQKLS
EEEFSAALLHLAKRRGVHNVNEVEEDTGNELSTKEQISRNSKALEEKYVA
ELQLERLKKDGEVRGSINRFKTSDYVKEAKQLLKVQKAYHQLDQSFIDTY
IDLLETRRTYYEGPGEGSPFGWKDIKEWYEMLMGHCTYFPEELRSVKYAY
NADLYNALNDLNNLVITRDENEKLEYYEKFQIIENVFKQKKKPTLKQIAK
EILVNEEDIKGYRVTSTGKPEFTNLKVYHDIKDITARKEIIENAELLDQI
AKILTIYQSSEDIQEELTNLNSELTQEEIEQISNLKGYTGTHNLSLKAIN
LILDELWHTNDNQIAIFNRLKLVPKKVDLSQQKEIPTTLVDDFILSPVVK
RSFIQSIKVINAIIKKYGLPNDIIIELAREKNSKDAQKMINEMQKRNRQT
NERIEEIIRTTGKENAKYLIEKIKLHDMQEGKCLYSLEAIPLEDLLNNPF
NYEVDHIIPRSVSFDNSFNNKVLVKQEENSKKGNRTPFQYLSSSDSKISY
ETFKKHILNLAKGKGRISKTKKEYLLEERDINRFSVQKDFINRNLVDTRY
ATRGLMNLLRSYFRVNNLDVKVKSINGGFTSFLRRKWKFKKERNKGYKHH
AEDALIIANADFIFKEWKKLDKAKKVMENQMFEEKQAESMPEIETEQEYK
EIFITPHQIKHIKDFKDYKYSHRVDKKPNRELINDTLYSTRKDDKGNTLI
VNNLNGLYDKDNDKLKKLINKSPEKLLMYHHDPQTYQKLKLIMEQYGDEK
NPLYKYYEETGNYLTKYSKKDNGPVIKKIKYYGNKLNAHLDITDDYPNSR
NKVVKLSLKPYRFDVYLDNGVYKFVTVKNLDVIKKENYYEVNSKCYEEAK
KLKKISNQAEFIASFYNNDLIKINGELYRVIGVNNDLLNRIEVNMIDITY
REYLENMNDKRPPRIIKTIASKTQSIKKYSTDILGNLYEVKSKKHPQIIK
KG.

In some embodiments, Cas protein is a SluCas9 protein. In some embodiments, the SluCas9 comprises an amino acid sequence that is at least 80%, 85%, 90%, 91%, 92%, 93%, 94%, 95%, 96%, 97%, 98%, 99% or 100% identical to the sequence of SEQ ID NO: 65:

NQKFILGLDIGITSVGYGLIDYETKNIIDAGVRLFPEANVENNEGRRSKR
GSRRLKRRRIHRLERVKKLLEDYNLLDQSQIPQSTNPYAIRVKGLSEALS
KDELVIALLHIAKRRGIHKIDVIDSNDDVGNELSTKEQLNKNSKLLKDKF
VCQIQLERMNEGQVRGEKNRFKTADIIKEIIQLLNVQKNFHQLDENFINK
YIELVEMRREYFEGPGKGSPYGWEGDPKAWYETLMGHCTYFPDELRSVKY
AYSADLFNALNDLNNLVIQRDGLSKLEYHEKYHIIENVFKQKKKPTLKQI
ANEINVNPEDIKGYRITKSGKPQFTEFKLYHDLKSVLFDQSILENEDVLD
QIAEILTIYQDKDSIKSKLTELDILLNEEDKENIAQLTGYTGTHRLSLKC
IRLVLEEQWYSSRNQMEIFTHLNIKPKKINLTAANKIPKAMIDEFILSPV
VKRTFGQAINLINKIIEKYGVPEDIIIELARENNSKDKQKFINEMQKKNE
NTRKRINEIIGKYGNQNAKRLVEKIRLHDEQEGKCLYSLESIPLEDLLNN
PNHYEVDHIIPRSVSFDNSYHNKVLVKQSENSKKSNLTPYQYFNSGKSKL
SYNQFKQHILNLSKSQDRISKKKKEYLLEERDINKFEVQKEFINRNLVDT
RYATRELTNYLKAYFSANNMNVKVKTINGSFTDYLRKVWKFKKERNHGYK
HHAEDALIIANADFLFKENKKLKAVNSVLEKPEIETKQLDIQVDSEDNYS
EMFIIPKQVQDIKDFRNFKYSHRVDKKPNRQLINDTLYSTRKKDNSTYIV
QTIKDIYAKDNTTLKKQFDKSPEKFLMYQHDPRTFEKLEVIMKQYANEKN
PLAKYHEETGEYLTKYSKKNNGPIVKSLKYIGNKLGSHLDVTHQFKSSTK
KLVKLSIKPYRFDVYLTDKGYKFITISYLDVLKKDNYYYIPEQKYDKLKL
GKAIDKNAKFIASFYKNDLIKLDGEIYKIIGVNSDTRNMIELDLPDIRYK
EYCELNNIKGEPRIKKTIGKKVNSIEKLTTDVLGNVFTNTQYTKPQLLFK
RGN.

In some embodiments, the Cas protein is any of the engineered Cas proteins disclosed in Schmidt et al., 2021, Nature Communications, “Improved CRISPR genome editing using small highly active and specific engineered RNA-guided nucleases.”

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 66 (designated herein as sRGN1):

MNQKFILGLDIGITSVGYGLIDYETKNIIDAGVRLFPEANVENNEGRRSK
RGSRRLKRRRIHRLDRVKHLLAEYDLLDLTNIPKSTNPYQTRVKGLNEKL
SKDELVIALLHIAKRRGIHNVDVAADKEETASDSLSTKDQINKNAKFLES
RYVCELQKERLENEGHVRGVENRFLTKDIVREAKKIIDTQMQYYPEIDET
FKEKYISLVETRREYFEGPGKGSPFGWEGNIKKWFEQMMGHCTYFPEELR
SVKYSYSAELFNALNDLNNLVITRDEDAKLNYGEKFQIIENVFKQKKTPN
LKQIAIEIGVHETEIKGYRVNKSGTPEFTEFKLYHDLKSIVFDKSILENE
AILDQIAEILTIYQDEQSIKEELNKLPEILNEQDKAEIAKLIGYNGTHRL
SLKCIHLINEELWQTSRNQMEIFNYLNIKPNKVDLSEQNKIPKDMVNDFI
LSPVVKRTFIQSINVINKVIEKYGIPEDIIIELARENNSDDRKKFINNLQ
KKNEATRKRINEIIGQTGNQNAKRIVEKIRLHDQQEGKCLYSLKDIPLED
LLRNPNNYDIDHIIPRSVSFDDSMHNKVLVRREQNAKKNNQTPYQYLTSG
YADIKYSVFKQHVLNLAENKDRMTKKKREYLLEERDINKFEVQKEFINRN
LVDTRYATRELTNYLKAYFSANNMNVKVKTINGSFTDYLRKVWKFKKERN
HGYKHHAEDALIIANADFLFKENKKLKAVNSVLEKPEIETKQLDIQVDSE
DNYSEMFIIPKQVQDIKDFRNFKYSHRVDKKPNRQLINDTLYSTRKKDNS
TYIVQTIKDIYAKDNTTLKKQFDKSPEKFLMYQHDPRTFEKLEVIMKQYA
NEKNPLAKYHEETGEYLTKYSKKNNGPIVKSLKYIGNKLGSHLDVTHQFK
SSTKKLVKLSIKPYRFDVYLTDKGYKFITISYLDVLKKDNYYYIPEQKYD
KLKLGKAIDKNAKFIASFYKNDLIKLDGEIYKIIGVNSDTRNMIELDLPD
IRYKEYCELNNIKGEPRIKKTIGKKVNSIEKLTTDVLGNVFTNTQYTKPQ
LLFKRGN.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 67 (designated herein as sRGN2):

MNQKFILGLDIGITSVGYGLIDYETKNIIDAGVRLFPEANVENNEGRRSK
RGSRRLKRRRIHRLERVKSLLSEYKIISGLAPTNNQPYNIRVKGLTEQLT
KDELAVALLHIAKRRGIHKIDVIDSNDDVGNELSTKEQLNKNSKLLKDKF
VCQIQLERMNEGQVRGEKNRFKTADIIKEIIQLLNVQKNFHQLDENFINK
YIELVEMRREYFEGPGQGSPFGWNGDLKKWYEMLMGHCTYFPQELRSVKY
AYSADLFNALNDLNNLIIQRDNSEKLEYHEKYHIIENVFKQKKKPTLKQI
AKEIGVNPEDIKGYRITKSGTPEFTEFKLYHDLKSVLFDQSILENEDVLD
QIAEILTIYQDKDSIKSKLTELDILLNEEDKENIAQLTGYNGTHRLSLKC
IRLVLEEQWYSSRNQMEIFTHLNIKPKKINLTAANKIPKAMIDEFILSPV
VKRTFIQSINVINKVIEKYGIPEDIIIELARENNSDDRKKFINNLQKKNE
ATRKRINEIIGQTGNQNAKRIVEKIRLHDQQEGKCLYSLESIALMDLLNN
PQNYEVDHIIPRSVAFDNSIHNKVLVKQIENSKKGNRTPYQYLNSSDAKL
SYNQFKQHILNLSKSKDRISKKKKDYLLEERDINKFEVQKEFINRNLVDT
RYATRELTSYLKAYFSANNMDVKVKTINGSFTNHLRKVWRFDKYRNHGYK
HHAEDALIIANADFLFKENKKLKAVNSVLEKPEIETKQLDIQVDSEDNYS
EMFIIPKQVQDIKDFRNFKYSHRVDKKPNRQLINDTLYSTRKKDNSTYIV
QTIKDIYAKDNTTLKKQFDKSPEKFLMYQHDPRTFEKLEVIMKQYANEKN
PLAKYHEETGEYLTKYSKKNNGPIVKSLKYIGNKLGSHLDVTHQFKSSTK
KLVKLSIKPYRFDVYLTDKGYKFITISYLDVLKKDNYYYIPEQKYDKLKL
GKAIDKNAKFIASFYKNDLIKLDGEIYKIIGVNSDTRNMIELDLPDIRYK
EYCELNNIKGEPRIKKTIGKKVNSIEKLTTDVLGNVFTNTQYTKPQLLFK
RGN.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 68 (designated herein as sRGN3):

MNQKFILGLDIGITSVGYGLIDYETKNIIDAGVRLFPEANVENNEGRRSK
RGSRRLKRRRIHRLERVKLLLTEYDLINKEQIPTSNNPYQIRVKGLSEIL
SKDELAIALLHLAKRRGIHNVDVAADKEETASDSLSTKDQINKNAKFLES
RYVCELQKERLENEGHVRGVENRFLTKDIVREAKKIIDTQMQYYPEIDET
FKEKYISLVETRREYFEGPGQGSPFGWNGDLKKWYEMLMGHCTYFPQELR
SVKYAYSADLFNALNDLNNLIIQRDNSEKLEYHEKYHIIENVFKQKKKPT
LKQIAKEIGVNPEDIKGYRITKSGTPEFTSFKLFHDLKKVVKDHAILDDI
DLLNQIAEILTIYQDKDSIVAELGQLEYLMSEADKQSISELTGYTGTHSL
SLKCMNMIIDELWHSSMNQMEVFTYLNMRPKKYELKGYQRIPTDMIDDAI
LSPVVKRTFIQSINVINKVIEKYGIPEDIIIELARENNSDDRKKFINNLQ
KKNEATRKRINEIIGQTGNQNAKRIVEKIRLHDQQEGKCLYSLESIPLED
LLNNPNHYEVDHIIPRSVSFDNSYHNKVLVKQSENSKKSNLTPYQYFNSG
KSKLSYNQFKQHILNLSKSQDRISKKKKEYLLEERDINKFEVQKEFINRN
LVDTRYATRELTNYLKAYFSANNMNVKVKTINGSFTDYLRKVWKFKKERN
HGYKHHAEDALIIANADFLFKENKKLKAVNSVLEKPEIETKQLDIQVDSE
DNYSEMFIIPKQVQDIKDFRNFKYSHRVDKKPNRQLINDTLYSTRKKDNS
TYIVQTIKDIYAKDNTTLKKQFDKSPEKFLMYQHDPRTFEKLEVIMKQYA
NEKNPLAKYHEETGEYLTKYSKKNNGPIVKSLKYIGNKLGSHLDVTHQFK
SSTKKLVKLSIKPYRFDVYLTDKGYKFITISYLDVLKKDNYYYIPEQKYD
KLKLGKAIDKNAKFIASFYKNDLIKLDGEIYKIIGVNSDTRNMIELDLPD
IRYKEYCELNNIKGEPRIKKTIGKKVNSIEKLTTDVLGNVFTNTQYTKPQ
LLFKRGN.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 69 (designated herein as sRGN3.1):

MNQKFILGLDIGITSVGYGLIDYETKNIIDAGVRLFPEANVENNEGRRSK
RGSRRLKRRRIHRLERVKLLLTEYDLINKEQIPTSNNPYQIRVKGLSEIL
SKDELAIALLHLAKRRGIHNVDVAADKEETASDSLSTKDQINKNAKFLES
RYVCELQKERLENEGHVRGVENRFLTKDIVREAKKIIDTQMQYYPEIDET
FKEKYISLVETRREYFEGPGQGSPFGWNGDLKKWYEMLMGHCTYFPQELR
SVKYAYSADLFNALNDLNNLIIQRDNSEKLEYHEKYHIIENVFKQKKKPT
LKQIAKEIGVNPEDIKGYRITKSGTPEFTSFKLFHDLKKVVKDHAILDDI
DLLNQIAEILTIYQDKDSIVAELGQLEYLMSEADKQSISELTGYTGTHSL
SLKCMNMIIDELWHSSMNQMEVFTYLNMRPKKYELKGYQRIPTDMIDDAI
LSPVVKRTFIQSINVINKVIEKYGIPEDIIIELARENNSDDRKKFINNLQ
KKNEATRKRINEIIGQTGNQNAKRIVEKIRLHDQQEGKCLYSLESIPLED
LLNNPNHYEVDHIIPRSVSFDNSYHNKVLVKQSENSKKSNLTPYQYFNSG
KSKLSYNQFKQHILNLSKSQDRISKKKKEYLLEERDINKFEVQKEFINRN
LVDTRYATRELTNYLKAYFSANNMNVKVKTINGSFTDYLRKVWKFKKERN
HGYKHHAEDALIIANADFLFKENKKLKAVNSVLEKPEIETKQLDIQVDSE
DNYSEMFIIPKQVQDIKDFRNFKYSHRVDKKPNRQLINDTLYSTRKKDNS
TYIVQTIKDIYAKDNTTLKKQFDKSPEKFLMYQHDPRTFEKLEVIMKQYA
NEKNPLAKYHEETGEYLTKYSKKNNGPIVKSLKYIGNKLGSHLDVTHQFK
SSTKKLVKLSIKNYRFDVYLTEKGYKFVTIAYLNVFKKDNYYYIPKDKYQ
ELKEKKKIKDTDQFIASFYKNDLIKLNGDLYKIIGVNSDDRNIIELDYYD
IKYKDYCEINNIKGEPRIKKTIGKKTESIEKFTTDVLGNLYLHSTEKAPQ
LIFKRGL.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 70 (designated herein as sRGN3.2):

MNQKFILGLDIGITSVGYGLIDYETKNIIDAGVRLFPEANVENNEGRRSK
RGSRRLKRRRIHRLERVKLLLTEYDLINKEQIPTSNNPYQIRVKGLSEIL
SKDELAIALLHLAKRRGIHNVDVAADKEETASDSLSTKDQINKNAKFLES
RYVCELQKERLENEGHVRGVENRFLTKDIVREAKKIIDTQMQYYPEIDET
FKEKYISLVETRREYFEGPGQGSPFGWNGDLKKWYEMLMGHCTYFPQELR
SVKYAYSADLFNALNDLNNLIIQRDNSEKLEYHEKYHIIENVFKQKKKPT
LKQIAKEIGVNPEDIKGYRITKSGTPEFTSFKLFHDLKKVVKDHAILDDI
DLLNQIAEILTIYQDKDSIVAELGQLEYLMSEADKQSISELTGYTGTHSL
SLKCMNMIIDELWHSSMNQMEVFTYLNMRPKKYELKGYQRIPTDMIDDAI
LSPVVKRTFIQSINVINKVIEKYGIPEDIIIELARENNSDDRKKFINNLQ
KKNEATRKRINEIIGQTGNQNAKRIVEKIRLHDQQEGKCLYSLESIPLED
LLNNPNHYEVDHIIPRSVSFDNSYHNKVLVKQSENSKKSNLTPYQYFNSG
KSKLSYNQFKQHILNLSKSQDRISKKKKEYLLEERDINKFEVQKEFINRN
LVDTRYATRELTNYLKAYFSANNMNVKVKTINGSFTDYLRKVWKFKKERN
HGYKHHAEDALIIANADFLFKENKKLKAVNSVLEKPEIETKQLDIQVDSE
DNYSEMFIIPKQVQDIKDFRNFKFSHRVDKKPNRQLINDTLYSTRMKDEH
DYIVQTITDIYGKDNTNLKKQFNKNPEKFLMYQNDPKTFEKLSIIMKQYS
DEKNPLAKYYEETGEYLTKYSKKNNGPIVKKIKLLGNKVGNHLDVTNKYE
NSTKKLVKLSIKNYRFDVYLTEKGYKFVTIAYLNVFKKDNYYYIPKDKYQ
ELKEKKKIKDTDQFIASFYKNDLIKLNGDLYKIIGVNSDDRNIIELDYYD
IKYKDYCEINNIKGEPRIKKTIGKKTESIEKFTTDVLGNLYLHSTEKAPQ
LIFKRGL.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 71 (designated herein as sRGN3.3):

MNQKFILGLDIGITSVGYGLIDYETKNIIDAGVRLFPEANVENNEGRRSK
RGSRRLKRRRIHRLERVKLLLTEYDLINKEQIPTSNNPYQIRVKGLSEIL
SKDELAIALLHLAKRRGIHNVDVAADKEETASDSLSTKDQINKNAKFLES
RYVCELQKERLENEGHVRGVENRFLTKDIVREAKKIIDTQMQYYPEIDET
FKEKYISLVETRREYFEGPGQGSPFGWNGDLKKWYEMLMGHCTYFPQELR
SVKYAYSADLFNALNDLNNLIIQRDNSEKLEYHEKYHIIENVFKQKKKPT
LKQIAKEIGVNPEDIKGYRITKSGTPEFTSFKLFHDLKKVVKDHAILDDI
DLLNQIAEILTIYQDKDSIVAELGQLEYLMSEADKQSISELTGYTGTHSL
SLKCMNMIIDELWHSSMNQMEVFTYLNMRPKKYELKGYQRIPTDMIDDAI
LSPVVKRTFIQSINVINKVIEKYGIPEDIIIELARENNSDDRKKFINNLQ
KKNEATRKRINEIIGQTGNQNAKRIVEKIRLHDQQEGKCLYSLESIPLED
LLNNPNHYEVDHIIPRSVSFDNSYHNKVLVKQSENSKKSNLTPYQYFNSG
KSKLSYNQFKQHILNLSKSQDRISKKKKEYLLEERDINKFEVQKEFINRN
LVDTRYATRELTSYLKAYFSANNMDVKVKTINGSFTNHLRKVWRFDKYRN
HGYKHHAEDALIIANADFLFKENKKLQNTNKILEKPTIENNTKKVTVEKE
EDYNNVFETPKLVEDIKQYRDYKFSHRVDKKPNRQLINDTLYSTRMKDEH
DYIVQTITDIYGKDNTNLKKQFNKNPEKFLMYQNDPKTFEKLSIIMKQYS
DEKNPLAKYYEETGEYLTKYSKKNNGPIVKKIKLLGNKVGNHLDVTNKYE
NSTKKLVKLSIKNYRFDVYLTEKGYKFVTIAYLNVFKKDNYYYIPKDKYQ
ELKEKKKIKDTDQFIASFYKNDLIKLNGDLYKIIGVNSDDRNIIELDYYD
IKYKDYCEINNIKGEPRIKKTIGKKTESIEKFTTDVLGNLYLHSTEKAPQ
LIFKRGL.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 72 (designated herein as sRGN4):

MNQKFILGLDIGITSVGYGLIDYETKNIIDAGVRLFPEANVENNEGRRSK
RGSRRLKRRRIHRLERVKKLLEDYNLLDQSQIPQSTNPYAIRVKGLSEAL
SKDELVIALLHIAKRRGIHNINVSSEDEDASNELSTKEQINRNNKLLKDK
YVCEVQLQRLKEGQIRGEKNRFKTTDILKEIDQLLKVQKDYHNLDIDFIN
QYKEIVETRREYFEGPGKGSPYGWEGDPKAWYETLMGHCTYFPDELRSVK
YAYSADLFNALNDLNNLVIQRDGLSKLEYHEKYHIIENVFKQKKKPTLKQ
IANEINVNPEDIKGYRITKSGKPEFTSFKLFHDLKKVVKDHAILDDIDLL
NQIAEILTIYQDKDSIVAELGQLEYLMSEADKQSISELTGYTGTHSLSLK
CMNMIIDELWHSSMNQMEVFTYLNMRPKKYELKGYQRIPTDMIDDAILSP
VVKRTFIQSINVINKVIEKYGIPEDIIIELARENNSDDRKKFINNLQKKN
EATRKRINEIIGQTGNQNAKRIVEKIRLHDQQEGKCLYSLESIPLEDLLN
NPNHYEVDHIIPRSVSFDNSYHNKVLVKQSENSKKSNLTPYQYFNSGKSK
LSYNQFKQHILNLSKSQDRISKKKKEYLLEERDINKFEVQKEFINRNLVD
TRYATRELTNYLKAYFSANNMNVKVKTINGSFTDYLRKVWKFKKERNHGY
KHHAEDALIIANADFLFKENKKLKAVNSVLEKPEIETKQLDIQVDSEDNY
SEMFIIPKQVQDIKDFRNFKYSHRVDKKPNRQLINDTLYSTRKKDNSTYI
VQTIKDIYAKDNTTLKKQFDKSPEKFLMYQHDPRTFEKLEVIMKQYANEK
NPLAKYHEETGEYLTKYSKKNNGPIVKSLKYIGNKLGSHLDVTHQFKSST
KKLVKLSIKPYRFDVYLTDKGYKFITISYLDVLKKDNYYYIPEQKYDKLK
LGKAIDKNAKFIASFYKNDLIKLDGEIYKIIGVNSDTRNMIELDLPDIRY
KEYCELNNIKGEPRIKKTIGKKVNSIEKLTTDVLGNVFTNTQYTKPQLLF
KRGN.

In some embodiments, the Cas9 nuclease lacks one or more amino acids from the range of amino acids corresponding to amino acids 1-4, 1-6, 77-85, 124-126, 124-129, 124-143, 124-145, 131-134, 135-138, 122-129, 505-622, 687-688, 716-720, 718-719, 718-730, 765, and/or 865-873 of SEQ ID NO: 2. In some embodiments, the Cas9 nuclease lacks the amino acids corresponding to amino acids 1-4, 1-6, 77-85, 124-126, 124-129, 124-143, 124-145, 131-134, 135-138, 122-129, 505-622, 687-688, 716-720, 718-719, 718-730, 765, and/or 865-873 of SEQ ID NO: 2. In some embodiments, the Cas9 nuclease lacks one or more amino acids from the range of amino acids corresponding to amino acids 1-4 of SEQ ID NO: 2. In some embodiments, the Cas9 nuclease lacks one or more amino acids from the range of amino acids corresponding to amino acids 1-6 of SEQ ID NO: 2. In some embodiments, the Cas9 nuclease lacks one or more amino acids from the range of amino acids corresponding to amino acids 77-85 of SEQ ID NO: 2. In some embodiments, the Cas9 nuclease lacks one or more amino acids from the range of amino acids corresponding to amino acids 124-126 of SEQ ID NO: 2. In some embodiments, the Cas9 nuclease lacks one or more amino acids from the range of amino acids corresponding to amino acids 124-129 of SEQ ID NO: 2. In some embodiments, the Cas9 nuclease lacks one or more amino acids from the range of amino acids corresponding to amino acids 124-143 of SEQ ID NO: 2. In some embodiments, the Cas9 nuclease lacks one or more amino acids from the range of amino acids corresponding to amino acids 124-145 of SEQ ID NO: 2. In some embodiments, the Cas9 nuclease lacks one or more amino acids from the range of amino acids corresponding to amino acids 131-134 of SEQ ID NO: 2. In some embodiments, the Cas9 nuclease lacks one or more amino acids from the range of amino acids corresponding to amino acids 135-138 of SEQ ID NO: 2. In some embodiments, the Cas9 nuclease lacks one or more amino acids from the range of amino acids corresponding to amino acids 122-129 of SEQ ID NO: 2. In some embodiments, the Cas9 nuclease lacks one or more amino acids from the range of amino acids corresponding to amino acids 505-622 of SEQ ID NO: 2. In some embodiments, the Cas9 nuclease lacks one or more amino acids from the range of amino acids corresponding to amino acids 687-688 of SEQ ID NO: 2. In some embodiments, the Cas9 nuclease lacks one or more amino acids from the range of amino acids corresponding to amino acids 716-720 of SEQ ID NO: 2. In some embodiments, the Cas9 nuclease lacks one or more amino acids from the range of amino acids corresponding to amino acids 718-719 of SEQ ID NO: 2. In some embodiments, the Cas9 nuclease lacks one or more amino acids from the range of amino acids corresponding to amino acids 718-730 of SEQ ID NO: 2. In some embodiments, the Cas9 nuclease lacks the amino acid corresponding to amino acid 765 of SEQ ID NO: 2. In some embodiments, the Cas9 nuclease lacks one or more amino acids from the range of amino acids corresponding to amino acids 865-873 of SEQ ID NO: 2. In some embodiments, the Cas9 nuclease lacks the amino acids corresponding to amino acids: 718-719 and 765 of SEQ ID NO: 2. In some embodiments, the Cas9 nuclease lacks the amino acids corresponding to amino acids 718-719 and 687-688 of SEQ ID NO: 2. In some embodiments, the Cas9 nuclease lacks the amino acids corresponding to amino acids 716-720 and 687-688 of SEQ ID NO: 2. In some embodiments, the Cas9 nuclease lacks the amino acids corresponding to amino acids 1-4 and 718-730 of SEQ ID NO: 2. In some embodiments, the Cas9 nuclease lacks the amino acids corresponding to amino acids 1-4, 687-688, and 716-720 of SEQ ID NO: 2. In some embodiments, the Cas9 nuclease lacks the amino acids corresponding to amino acids 1-4 and 124-145 of SEQ ID NO: 2. In some embodiments, the Cas9 nuclease lacks the amino acids corresponding to amino acids 1-4, 505-622, and 718-730 of SEQ ID NO: 2. In some embodiments, the Cas9 nuclease does not lack one or more amino acids from the range of amino acids corresponding to amino acids: 1-8 of SEQ ID NO: 2, 1-10 of SEQ ID NO: 2, 81-94 of SEQ ID NO: 2, 163-164 of SEQ ID NO: 2, 181-184 of SEQ ID NO: 2, 244-424 of SEQ ID NO: 2, 714-769 of SEQ ID NO: 2, 736-742 of SEQ ID NO: 2, 978-979 of SEQ ID NO: 2, and/or 1039-1052 of SEQ ID NO: 2. In some embodiments, the Cas9 nuclease does not lack the amino acid ranges corresponding to amino acids: 1-8 of SEQ ID NO: 2, 1-10 of SEQ ID NO: 2, 81-94 of SEQ ID NO: 2, 163-164 of SEQ ID NO: 2, 181-184 of SEQ ID NO: 2, 244-424 of SEQ ID NO: 2, 714-769 of SEQ ID NO: 2, 736-742 of SEQ ID NO: 2, 978-979 of SEQ ID NO: 2, and/or 1039-1052 of SEQ ID NO: 2.

In some embodiments, the Cas9 nuclease lacks one or more amino acids from the range of amino acids corresponding to amino acids 81-94, 123-145, 244-424, 714-769, and/or 717-730 of SEQ ID NO: 2. In some embodiments, the Cas9 nuclease lacks the amino acids corresponding to amino acids 81-94, 123-145, 244-424, 714-769, and/or 717-730 of SEQ ID NO: 2. In some embodiments, the Cas9 nuclease lacks one or more amino acids from the range of amino acids corresponding to amino acids 81-94 of SEQ ID NO: 2. In some embodiments, the Cas9 nuclease lacks one or more amino acids from the range of amino acids corresponding to amino acids 123-145 of SEQ ID NO: 2. In some embodiments, the Cas9 nuclease lacks one or more amino acids from the range of amino acids corresponding to amino acids 244-424 of SEQ ID NO: 2. In some embodiments, the Cas9 nuclease lacks one or more amino acids from the range of amino acids corresponding to amino acids 714-769 of SEQ ID NO: 2. In some embodiments, the Cas9 nuclease lacks one or more amino acids from the range of amino acids corresponding to amino acids 717-730 of SEQ ID NO: 2.

In some embodiments, the Cas9 nuclease lacks one or more amino acids from the range of amino acids corresponding to amino acids 1-4, 82-85, 122-130, 124-147, 182-185, 720-722, 723-727, 723-733, 731-734, 977-980 and/or 1049-1053 of SEQ ID NO: 65. In some embodiments, the Cas9 nuclease lacks the amino acids corresponding to amino acids 1-4, 82-85, 122-130, 124-147, 182-185, 720-722, 723-727, 723-733, 731-734, 977-980 and/or 1049-1053 of SEQ ID NO: 2. In some embodiments, the Cas9 nuclease lacks one or more amino acids from the range of amino acids corresponding to amino acids 1-4 of SEQ ID NO: 65. In some embodiments, the Cas9 nuclease lacks one or more amino acids from the range of amino acids corresponding to amino acids 122-130 of SEQ ID NO: 65. In some embodiments, the Cas9 nuclease lacks one or more amino acids from the range of amino acids corresponding to amino acids 124-147 of SEQ ID NO: 65. In some embodiments, the Cas9 nuclease lacks one or more amino acids from the range of amino acids corresponding to amino acids 182-185 of SEQ ID NO: 65. In some embodiments, the Cas9 nuclease lacks one or more amino acids from the range of amino acids corresponding to amino acids 720-722 of SEQ ID NO: 65. In some embodiments, the Cas9 nuclease lacks one or more amino acids from the range of amino acids corresponding to amino acids 723-733 of SEQ ID NO: 65. In some embodiments, the Cas9 nuclease lacks one or more amino acids from the range of amino acids corresponding to amino acids 731-734 of SEQ ID NO: 65. In some embodiments, the Cas9 nuclease lacks one or more amino acids from the range of amino acids corresponding to amino acids 977-980 of SEQ ID NO: 65. In some embodiments, the Cas9 nuclease lacks one or more amino acids from the range of amino acids corresponding to amino acids 1049-1053 of SEQ ID NO: 65.

In some embodiments, a Cas protein comprising an amino acid sequence that is at least 80%, 85%, 90%, 91%, 92%, 93%, 94%, 95%, 96%, 97%, 98%, 99%, or 100% identical to the sequence of any one of SEQ ID Nos: 2 or 65-72 lacks one or more amino acids from the range of amino acids corresponding to (or homologous to) amino acids 1-4, 1-6, 77-85, 124-126, 124-129, 124-143, 124-145, 131-134, 135-138, 122-129, 505-622, 687-688, 716-720, 718-719, 718-730, 765, and/or 865-873 of SEQ ID NO: 2. In some embodiments, a Cas protein comprising an amino acid sequence that is at least 80%, 85%, 90%, 91%, 92%, 93%, 94%, 95%, 96%, 97%, 98%, 99%, or 100% identical to the sequence of any one of SEQ ID Nos: 2 or 65-72 lacks the amino acids corresponding to (or homologous to) amino acids 1-4, 1-6, 77-85, 124-126, 124-129, 124-143, 124-145, 131-134, 135-138, 122-129, 505-622, 687-688, 716-720, 718-719, 718-730, 765, and/or 865-873 of SEQ ID NO: 2. In some embodiments, a Cas protein comprising an amino acid sequence that is at least 80%, 85%, 90%, 91%, 92%, 93%, 94%, 95%, 96%, 97%, 98%, 99%, or 100% identical to the sequence of any one of SEQ ID Nos: 2 or 65-72 lacks the amino acids corresponding to (or homologous to) amino acids: 718-719 and 765 of SEQ ID NO: 2. In some embodiments, a Cas protein comprising an amino acid sequence that is at least 80%, 85%, 90%, 91%, 92%, 93%, 94%, 95%, 96%, 97%, 98%, 99%, or 100% identical to the sequence of any one of SEQ ID Nos: 2 or 65-72 lacks the amino acids corresponding to (or homologous to) amino acids 718-719 and 687-688 of SEQ ID NO: 2. In some embodiments, a Cas protein comprising an amino acid sequence that is at least 80%, 85%, 90%, 91%, 92%, 93%, 94%, 95%, 96%, 97%, 98%, 99%, or 100% identical to the sequence of any one of SEQ ID Nos: 2 or 65-72 lacks the amino acids corresponding to (or homologous to) amino acids 716-720 and 687-688 of SEQ ID NO: 2. In some embodiments, a Cas protein comprising an amino acid sequence that is at least 80%, 85%, 90%, 91%, 92%, 93%, 94%, 95%, 96%, 97%, 98%, 99%, or 100% identical to the sequence of any one of SEQ ID Nos: 2 or 65-72 lacks the amino acids corresponding to (or homologous to) amino acids 1-4 and 718-730 of SEQ ID NO: 2. In some embodiments, a Cas protein comprising an amino acid sequence that is at least 80%, 85%, 90%, 91%, 92%, 93%, 94%, 95%, 96%, 97%, 98%, 99%, or 100% identical to the sequence of any one of SEQ ID Nos: 2 or 65-72 lacks the amino acids corresponding to (or homologous to) amino acids 1-4, 687-688, and 716-720 of SEQ ID NO: 2. In some embodiments, a Cas protein comprising an amino acid sequence that is at least 80%, 85%, 90%, 91%, 92%, 93%, 94%, 95%, 96%, 97%, 98%, 99%, or 100% identical to the sequence of any one of SEQ ID Nos: 2 or 65-72 lacks the amino acids corresponding to (or homologous to) amino acids 1-4 and 124-145 of SEQ ID NO: 2. In some embodiments, a Cas protein comprising an amino acid sequence that is at least 80%, 85%, 90%, 91%, 92%, 93%, 94%, 95%, 96%, 97%, 98%, 99%, or 100% identical to the sequence of any one of SEQ ID Nos: 2 or 65-72 lacking the amino acids corresponding to (or homologous to) amino acids 1-4, 505-622, and 718-730 of SEQ ID NO: 2. In some embodiments, a Cas protein comprising an amino acid sequence that is at least 80%, 85%, 90%, 91%, 92%, 93%, 94%, 95%, 96%, 97%, 98%, 99%, or 100% identical to the sequence of any one of SEQ ID Nos: 2 or 65-72 does not lack one or more amino acids from the range of amino acids corresponding to (or homologous to) amino acids: 1-8 of SEQ ID NO: 2, 1-10 of SEQ ID NO: 2, 81-94 of SEQ ID NO: 2, 163-164 of SEQ ID NO: 2, 181-184 of SEQ ID NO: 2, 244-424 of SEQ ID NO: 2, 714-769 of SEQ ID NO: 2, 736-742 of SEQ ID NO: 2, 978-979 of SEQ ID NO: 2, and/or 1039-1052 of SEQ ID NO: 2. In some embodiments, a Cas protein comprising an amino acid sequence that is at least 80%, 85%, 90%, 91%, 92%, 93%, 94%, 95%, 96%, 97%, 98%, 99%, or 100% identical to the sequence of any one of SEQ ID Nos: 2 or 65-72 does not lack the amino acid ranges corresponding to (or homologous to) amino acids: 1-8 of SEQ ID NO: 2, 1-10 of SEQ ID NO: 2, 81-94 of SEQ ID NO: 2, 163-164 of SEQ ID NO: 2, 181-184 of SEQ ID NO: 2, 244-424 of SEQ ID NO: 2, 714-769 of SEQ ID NO: 2, 736-742 of SEQ ID NO: 2, 978-979 of SEQ ID NO: 2, and/or 1039-1052 of SEQ ID NO: 2.

In some embodiments, any of the truncated Cas proteins disclosed herein is capable of binding a target DNA sequence. In some embodiments, the truncated Cas protein is capable of binding a target DNA sequence when the truncated Cas protein is complexed with a gRNA comprising a sequence (e.g., a spacer sequence) that is complementary to the target DNA sequence. In some embodiments, the truncated Cas protein is capable of binding to a target sequence at least 10%, 20%, 30%, 40%, 50%, 60%, 70%, 80%, 90%, or 100% as efficiently as a reference untruncated Cas protein (e.g., a Cas protein comprising the amino acid sequence of SEQ ID NO: 2) for the same target sequence under the same conditions. In some embodiments, the truncated Cas protein is capable of binding to a target sequence 10-100%, 10-80%, 10-60%, 10-40%, 10-20%, 30-100%, 30-80%, 30-60%, 30-40%, 50-100%, 50-80%, 50-60%, 70-100%, 70-80%, 80-90%, 90-100%, or 85-95% as efficiently as a reference untruncated Cas protein (e.g., a Cas protein comprising the amino acid sequence of SEQ ID NO: 2) for the same target sequence under the same conditions. In some embodiments, the truncated Cas protein is capable of binding to a target sequence with a binding affinity that is at least 10%, 20%, 30%, 40%, 50%, 60%, 70%, 80%, 90%, or 100% as the binding affinity of a reference untruncated Cas protein (e.g., a Cas protein comprising the amino acid sequence of SEQ ID NO: 2) for the same target sequence under the same conditions. In some embodiments, the truncated Cas protein is capable of binding to a target sequence with a binding affinity that is 10-100%, 10-80%, 10-60%, 10-40%, 10-20%, 30-100%, 30-80%, 30-60%, 30-40%, 50-100%, 50-80%, 50-60%, 70-100%, 70-80%, 80-90%, 90-100%, or 85-95% of the binding affinity of a reference untruncated Cas protein (e.g., a Cas protein comprising the amino acid sequence of SEQ ID NO: 2) for the same target sequence under the same conditions.

In some embodiments, the Cas9 protein may be fused to a heterologous moiety. In some embodiments the heterologous moiety is a deaminase (e.g., TadA). In some embodiments, the heterologous moiety is a protein useful for gene silencing (e.g., a KRAB domain, Dnmt3A, and/or Dnmt3L protein domains). In some embodiments, the heterologous moiety is a DNA methyltransferase, e.g., Dnmt1, Dnmt3A, or Dnmt3B. In some embodiments, the heterologous moiety is a regulatory factor of a factor of DNA methyltransferase (e.g., a catalytically inactive regulatory factor of DNA methyltransferase (e.g., Dnmt3L)). In some embodiments, the heterologous moiety is a protein useful for reversing gene silencing (e.g., a TET1 DNA demethylase catalytic domain) In some embodiments, the heterologous moiety is a transcriptional repressor. In some embodiments, the heterologous moiety is a transcriptional activator.

In some embodiments, the compact protein comprises a KRAB domain, a category of transcriptional repression domains that are usually 45-75 amino acids in length. See, e.g., Ecco, G., Imbeault, M., Trono, D., KRAB zinc finger proteins, Development 144, 2017; Lambert et al. The human transcription factors, Cell 172, 2018. In some embodiments, the KRAB domain is a KRAB domain of Kox 1. In some embodiments, the KRAB domain comprises the sequence of SEQ ID NO: 73. In some embodiments, the KRAB domain comprises an amino acid sequence that is at least 50%, 55%, 60%, 65%, 70%, 75%, 80%, 85%, 90%, 91%, 92%, 93%, 94%, 95%, 96%, 97%, 98%, 99% or 100% identical to SEQ ID NO: 73.

In some embodiments, the compact protein comprises a DNA (cytosine-5)-methyltransferase 3A (Dnmt3A) protein or fragment thereof. In some embodiments, the Dnmt3A comprises the sequence of SEQ ID NO: 74. In some embodiments, the Dnmt3A comprises an amino acid sequence that is at least 50%, 55%, 60%, 65%, 70%, 75%, 80%, 85%, 90%, 91%, 92%, 93%, 94%, 95%, 96%, 97%, 98%, 99% or 100% identical to SEQ ID NO: 74.

In some embodiments, the compact protein comprises a DNA (cytosine-5)-methyltransferase 3L (Dnmt3L) protein or fragment thereof. In some embodiments, the Dnmt3L comprises the sequence of SEQ ID NO: 75. In some embodiments, the Dnmt3L comprises an amino acid sequence that is at least 50%, 55%, 60%, 65%, 70%, 75%, 80%, 85%, 90%, 91%, 92%, 93%, 94%, 95%, 96%, 97%, 98%, 99% or 100% identical to SEQ ID NO: 75.

### Nucleobase Editor Activity

In some embodiments, the nucleobase editor has at least 50%, 60%, 70%, 80%, or 90% base editing activity as compared to a nucleobase editor comprising the sequence of SEQ ID NO: 3. In some embodiments, the base editing activity is assessed using a polynucleotide comprising the nucleotide sequence of SEQ ID NO: 4. In some embodiments, the base editing activity is assessed at position A5, A8 or A13 of SEQ ID NO: 4.

In some embodiments, any of the proteins disclosed herein further comprises one or more nuclear localization signals (NLSs) and optionally one or more linkers fusing the one or more NLSs to any of the proteins disclosed herein (e.g., any of the nucleobase editors disclosed herein). In some embodiments, the one or more NLSs are selected from a c-myc NLS (e.g., an NLS comprising the amino acid sequence of SEQ ID NO: 5), an SV40 NLS (e.g., an NLS comprising the amino acid sequence of SEQ ID NO: 7), and/or a nucleoplasmin NLS (e.g., an NLS comprising the amino acid sequence of SEQ ID NO: 9). In some embodiments, the disclosure provides for a nucleobase editor comprising: a) a first NLS conjugated to the N-terminus of the nucleobase editor, optionally by means of a first NLS linker, and b) a second NLS conjugated to the C-terminus of the nucleobase editor, optionally by means of a second NLS linker. In some embodiments, the nucleobase editor further comprises: c) a third NLS conjugated to the second NLS, optionally by means of a third NLS linker.

In some embodiments, the first NLS comprises the amino acid sequence of SEQ ID NO: 5: PAAKKKKLD, wherein the optional first NLS linker comprises the amino acid sequence of SEQ ID NO: 6: GSVD, wherein the second NLS comprises the amino acid sequence of SEQ ID NO: 7: PKKKRKV, wherein the optional second NLS linker comprises the amino acid sequence of SEQ ID NO: 8: TGGGPGGGAAAGSGS, wherein the third NLS comprises the amino acid sequence of SEQ ID NO: 9: KRPAATKKAGQAKKKK, and wherein the optional third NLS linker comprises the amino acid sequence of SEQ ID NO: 10: GSGS. In some embodiments, the one or more NLSs and one or more linkers fusing the one or more NLSs to the nucleobase editor are collectively no more than 40 amino acids. In some embodiments, the one or more NLSs and one or more linkers fusing the one or more NLSs to the nucleobase editor are collectively no more than 37 amino acids. In some embodiments, the one or more NLSs and one or more linkers fusing the one or more NLSs to the nucleobase editor are collectively no more than 35 amino acids. In some embodiments, the one or more NLSs and one or more linkers fusing the one or more NLSs to the nucleobase editor are collectively no more than 32 amino acids. In some embodiments, the one or more NLSs and one or more linkers fusing the one or more NLSs to the nucleobase editor are collectively no more than 28 amino acids. In some embodiments, the one or more NLSs and one or more linkers fusing the one or more NLSs to the nucleobase editor are collectively no more than 26 amino acids. In some embodiments, the one or more NLSs and one or more linkers fusing the one or more NLSs to the nucleobase editor are collectively 10 to 40 amino acids in length. In some embodiments, the one or more NLSs and one or more linkers fusing the one or more NLSs to the nucleobase editor are collectively 11 to 37 amino acids in length. In some embodiments, the one or more NLSs and one or more linkers fusing the one or more NLSs to the nucleobase editor are collectively 13 to 35 amino acids in length. In some embodiments, the one or more NLSs and one or more linkers fusing the one or more NLSs to the nucleobase editor are collectively 15 to 32 amino acids in length. In some embodiments, the one or more NLSs and one or more linkers fusing the one or more NLSs to the nucleobase editor are collectively 16 to 30 amino acids in length. In some embodiments, the one or more NLSs and one or more linkers fusing the one or more NLSs to the nucleobase editor are collectively 18 to 28 amino acids in length. In some embodiments, the one or more NLSs and one or more linkers fusing the one or more NLSs to the nucleobase editor are collectively 20 to 26 amino acids in length.

In some embodiments, up to 8 amino acids are deleted from the amino terminus end of the nucleotide deaminase. In some embodiments, up to 8 amino acids are deleted from the carboxy terminus end of the nucleotide deaminase. In some embodiments, up to 8 amino acids are deleted from the carboxy terminus end of the nucleotide deaminase. In some embodiments, up to 22 amino acids are deleted from the REC-lobe domain of the Cas9 nuclease.

In some embodiments, the amino acid sequence of the linker is selected from the group of SEQ ID NO: 11, SEQ ID NO: 12, and SEQ ID NO: 13; up to 8 amino acids are deleted from the amino terminus end of the RNA adenine deaminase; up to 8 amino acids are deleted from the carboxy terminus end of the RNA adenine deaminase; and up to 22 amino acids are deleted from the REC-lobe domain of the Cas9 nuclease.

In some embodiments, the amino acid sequence which encodes the nucleobase editor is at least 80%, 85%, 90%, 91%, 92%, 93%, 94%, 95%, 96%, 97%, 98%, 99% or 100% identical to SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, or SEQ ID NO: 56. In some embodiments, the amino acid sequence which encodes the nucleobase editor is selected from the group SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, and SEQ ID NO: 56.

In some embodiments, the overall activity of the nucleobase editor is at least 30% or greater when compared to the overall activity of wild type nucleobase editor. In some embodiments, the overall activity of the nucleobase editor is at least 40% or greater when compared to the overall activity of wild type nucleobase editor. In some embodiments, the overall activity of the nucleobase editor is at least 50% or greater when compared to the overall activity of wild type nucleobase editor. In some embodiments, the overall activity of the nucleobase editor is at least 60% or greater when compared to the overall activity of wild type nucleobase editor. In some embodiments, the overall activity of the nucleobase editor is at least 70% or greater when compared to the overall activity of wild type nucleobase editor. In some embodiments, the overall activity of the nucleobase editor is at least 80% or greater when compared to the overall activity of wild type nucleobase editor. In some embodiments, the overall activity of the nucleobase editor is at least 90% or greater when compared to the overall activity of wild type nucleobase editor. In some embodiments, base editing activity is compared to a wild type nucleobase editor comprising the sequence of SEQ ID NO: 3. In some embodiments, the base editing activity is assessed using a polynucleotide comprising the nucleotide sequence of SEQ ID NO: 4. In some embodiments, the base editing activity is assessed at position A5, A8 or A13 of SEQ ID NO: 4: ATGCATTAACTGAAAATGGTCA.

Some aspects of the disclosure are directed to a nucleic acid encoding a nucleobase editor comprising a nucleotide deaminase, a Cas9 nuclease, and a linker between the deaminase and Cas9 nuclease, wherein the nucleobase editor is between 1175-1249 amino acids in length. In some embodiments, the nucleic acid further comprises inverted terminal repeats (ITRs). In some embodiments, the nucleic acid further comprises an sgRNA. In some embodiments, the nucleic acid further comprises promotor sequences.

In some embodiments, the entire nucleic acid from ITR to ITR, including the ITR, is less than 5 kb. In some embodiments, the entire nucleic acid from ITR to ITR, including the ITR, is less than 4.9 kb. In some embodiments, the entire nucleic acid from ITR to ITR, including the ITR, is less than 4.85 kb. In some embodiments, the entire nucleic acid from ITR to ITR, including the ITR, is less than 4.8 kb.

In some embodiments, the amino acid sequence which encodes the nucleobase editor is at least 80%, 85%, 90%, 91%, 92%, 93%, 94%, 95%, 96%, 97%, 98%, 99% or 100% identical to SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, or SEQ ID NO: 56. In some embodiments, the amino acid sequence which encodes the nucleobase editor is selected from the group SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, and SEQ ID NO: 56.

### Vectors

Some aspects of the disclosure are directed to a vector comprising a nucleic acid encoding a nucleobase editor comprising a nucleotide deaminase, a Cas9 nuclease, and a linker between the deaminase and Cas9 nuclease, wherein the nucleobase editor is between 1175-1249 amino acids in length. In some embodiments, the vector is selected from the group of adeno-associated virus, adenovirus, lentivirus, bacteriophage, and virus-like particles. In some embodiments, the vector is an adeno-associated virus.

### Isolated Cells

Certain aspects of the current disclosure are directed to an isolated cell comprising a nucleobase editor comprising a nucleotide deaminase, a Cas9 nuclease, and a linker between the deaminase and Cas9 nuclease, wherein the nucleobase editor is between 1175-1249 amino acids in length. In some embodiments, the isolated cell comprises a nucleic acid encoding a nucleobase editor comprising a nucleotide deaminase, a Cas9 nuclease, and a linker between the deaminase and Cas9 nuclease, wherein the nucleobase editor is between 1175-1249 amino acids in length. In some embodiments, the isolated cell comprises a nucleic acid encoding a nucleobase editor comprising a nucleotide deaminase, a Cas9 nuclease, and a linker between the deaminase and Cas9 nuclease, wherein the nucleobase editor is between 1175-1249 amino acids in length, wherein the nucleic acid further comprises inverted terminal repeats (ITRs). In some embodiments, the isolated cell comprises a nucleic acid encoding a nucleobase editor comprising a nucleotide deaminase, a Cas9 nuclease, and a linker between the deaminase and Cas9 nuclease, wherein the nucleobase editor is between 1175-1249 amino acids in length, wherein the nucleic acid further comprises ITRs, and a sgRNA. In some embodiments, the isolated cell comprises a nucleic acid encoding a nucleobase editor comprising a nucleotide deaminase, a Cas9 nuclease, and a linker between the deaminase and Cas9 nuclease, wherein the nucleobase editor is between 1175-1249 amino acids in length, wherein the nucleic acid further comprises ITRs, a sgRNA, and promoter sequences. In some embodiments, the isolated cell comprises a nucleic acid encoding a nucleobase editor comprising a nucleotide deaminase, a Cas9 nuclease, and a linker between the deaminase and Cas9 nuclease, wherein the nucleobase editor is between 1175-1249 amino acids in length, wherein the nucleic acid further comprises ITRs, a sgRNA, and promoter sequences and the entire nucleic acid from ITR to ITR, including the ITR, is less than 5 kb. In some embodiments, the isolated cell comprises a nucleic acid encoding a nucleobase editor comprising a nucleotide deaminase, a Cas9 nuclease, and a linker between the deaminase and Cas9 nuclease, wherein the nucleobase editor is between 1175-1249 amino acids in length, wherein the nucleic acid further comprises ITRs, a sgRNA, and promoter sequences and the entire nucleic acid from ITR to ITR, including the ITR, is less than 4.95 kb. In some embodiments, the isolated cell comprises a nucleic acid encoding a nucleobase editor comprising a nucleotide deaminase, a Cas9 nuclease, and a linker between the deaminase and Cas9 nuclease, wherein the nucleobase editor is between 1175-1249 amino acids in length, wherein the nucleic acid further comprises ITRs, a sgRNA, and promoter sequences and the entire nucleic acid from ITR to ITR, including the ITR, is less than 4.9 kb. In some embodiments, the isolated cell comprises a nucleic acid encoding a nucleobase editor comprising a nucleotide deaminase, a Cas9 nuclease, and a linker between the deaminase and Cas9 nuclease, wherein the nucleobase editor is between 1175-1249 amino acids in length, wherein the nucleic acid further comprises ITRs, a sgRNA, and promoter sequences and the entire nucleic acid from ITR to ITR, including the ITR, is less than 4.85 kb. In some embodiments, the isolated cell comprises a nucleic acid encoding a nucleobase editor comprising a nucleotide deaminase, a Cas9 nuclease, and a linker between the deaminase and Cas9 nuclease, wherein the nucleobase editor is between 1175-1249 amino acids in length, wherein the nucleic acid further comprises ITRs, a sgRNA, and promoter sequences and the entire nucleic acid from ITR to ITR, including the ITR, is less than 4.8 kb.

In some embodiments, the isolated cell comprises a vector comprising a nucleic acid encoding a nucleobase editor comprising a nucleotide deaminase, a Cas9 nuclease, and a linker between the deaminase and Cas9 nuclease, wherein the nucleobase editor is between 1175-1249 amino acids in length. In some embodiments, the isolated cell comprises a vector comprising a nucleic acid encoding a nucleobase editor comprising a nucleotide deaminase, a Cas9 nuclease, and a linker between the deaminase and Cas9 nuclease, wherein the nucleobase editor is between 1175-1249 amino acids in length, wherein the nucleic acid further comprises inverted terminal repeats (ITRs). In some embodiments, the isolated cell comprises a vector comprising a nucleic acid encoding a nucleobase editor comprising a nucleotide deaminase, a Cas9 nuclease, and a linker between the deaminase and Cas9 nuclease, wherein the nucleobase editor is between 1175-1249 amino acids in length, wherein the nucleic acid further comprises ITRs, and a sgRNA. In some embodiments, the isolated cell comprises a vector comprising a nucleic acid encoding a nucleobase editor comprising a nucleotide deaminase, a Cas9 nuclease, and a linker between the deaminase and Cas9 nuclease, wherein the nucleobase editor is between 1175-1249 amino acids in length, wherein the nucleic acid further comprises ITRs, a sgRNA, and promoter sequences. In some embodiments, the isolated cell comprises a vector comprising a nucleic acid encoding a nucleobase editor comprising a nucleotide deaminase, a Cas9 nuclease, and a linker between the deaminase and Cas9 nuclease, wherein the nucleobase editor is between 1175-1249 amino acids in length, wherein the nucleic acid further comprises ITRs, a sgRNA, and promoter sequences and the entire nucleic acid from ITR to ITR, including the ITR, is less than 5 kb. In some embodiments, the isolated cell comprises a vector comprising a nucleic acid encoding a nucleobase editor comprising a nucleotide deaminase, a Cas9 nuclease, and a linker between the deaminase and Cas9 nuclease, wherein the nucleobase editor is between 1175-1249 amino acids in length, wherein the nucleic acid further comprises ITRs, a sgRNA, and promoter sequences and the entire nucleic acid from ITR to ITR, including the ITR, is less than 4.95 kb. In some embodiments, the isolated cell comprises a vector comprising a nucleic acid encoding a nucleobase editor comprising a nucleotide deaminase, a Cas9 nuclease, and a linker between the deaminase and Cas9 nuclease, wherein the nucleobase editor is between 1175-1249 amino acids in length, wherein the nucleic acid further comprises ITRs, a sgRNA, and promoter sequences and the entire nucleic acid from ITR to ITR, including the ITR, is less than 4.9 kb. In some embodiments, the isolated cell comprises a vector comprising a nucleic acid encoding a nucleobase editor comprising a nucleotide deaminase, a Cas9 nuclease, and a linker between the deaminase and Cas9 nuclease, wherein the nucleobase editor is between 1175-1249 amino acids in length, wherein the nucleic acid further comprises ITRs, a sgRNA, and promoter sequences and the entire nucleic acid from ITR to ITR, including the ITR, is less than 4.85 kb. In some embodiments, the isolated cell comprises a vector comprising a nucleic acid encoding a nucleobase editor comprising a nucleotide deaminase, a Cas9 nuclease, and a linker between the deaminase and Cas9 nuclease, wherein the nucleobase editor is between 1175-1249 amino acids in length, wherein the nucleic acid further comprises ITRs, a sgRNA, and promoter sequences and the entire nucleic acid from ITR to ITR, including the ITR, is less than 4.8 kb.

### Methods of Treatment

The term “treatment” or “treating” refers to preventing or delaying the onset, slowing down the progression, and/or or ameliorating the symptoms of the disorder. In some embodiments, any of the proteins disclosed herein (e.g., any of the nucleobase editors disclosed herein) may be administered to a subject. In some embodiments, the subject has a disease. In some embodiments, the subject has a liver disease. In some embodiments, the subject has a neurological disorder.

### EXAMPLES

The following examples are presented to illustrate the present disclosure. The examples are not intended to be limiting in any manner.

### Experimental Procedures for Examples

Base editor activity was assessed in HEK293FT cells. The base editor variants and appropriate sgRNAs were delivered via a single plasmid into cells. Base editor expression was regulated by a CMV-promoter and sgRNA expression was regulated by a U6 promoter. Base editors were transfected into HEK293FT cells using Lipofectamine 2000 Transfection Reagent (ThermoFisher Scientific, 11668019) and incubated at 37° C. After 3 days, cells were lysed and genomic DNA was extracted using Lucigen QuickExtract DNA Extraction Solution. Genomic DNA was analyzed by either high-throughput DNA sequencing or Sanger sequencing at the targeted sites. Cellular A to G conversion percentages were determined.

### Example 1: Shortened Linker Between Deaminase and Cas9 Nuclease

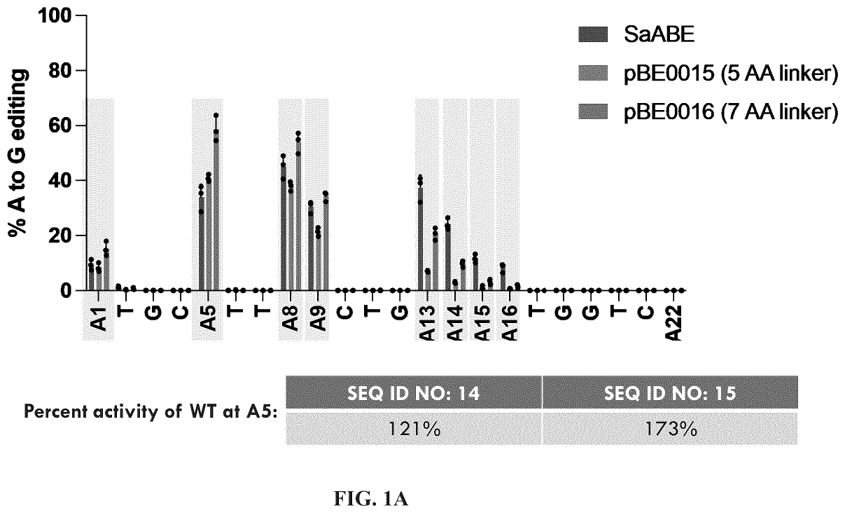

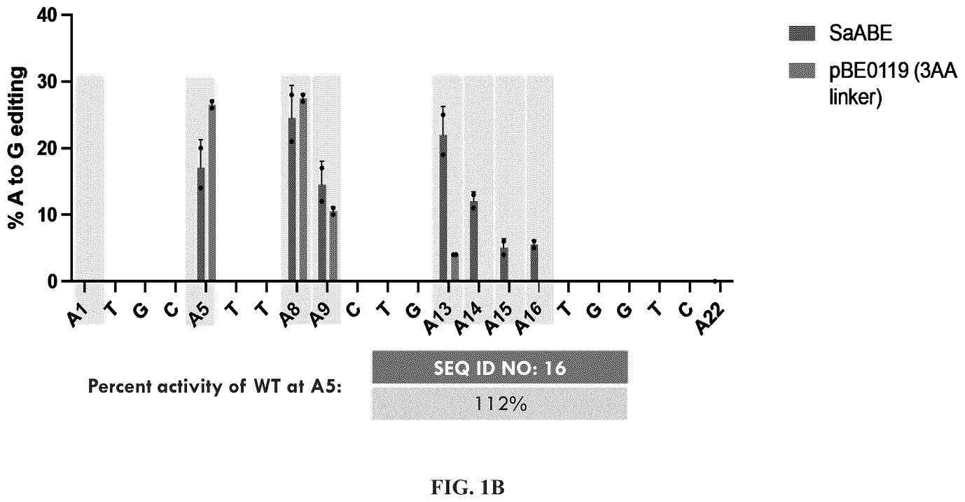

The linker between the TadA-8e and nSaCas9 was trimmed from 16 amino acids to 3 amino acids (SEQ ID NO: 11), 5 amino acids (SEQ ID NO: 12), or 7 amino acids (SEQ ID NO: 13). All three linker replacements were well-tolerated as shown in FIG. 1A, FIG. 1B, and Table 1. Nucleobase activity was compared to that of the wild type SaABE nucleobase editor. All three nucleobase editors with linker reductions showed nucleobase activity greater than 100% of wild type nucleobase editor activity. However, all three linker replacements resulted in a narrowed-editing window. In some embodiments, a narrowed-editing window may be advantageous for certain applications when multiple A to G edits are undesired.

### Example 2: Removal of Amino Acids From Deaminase

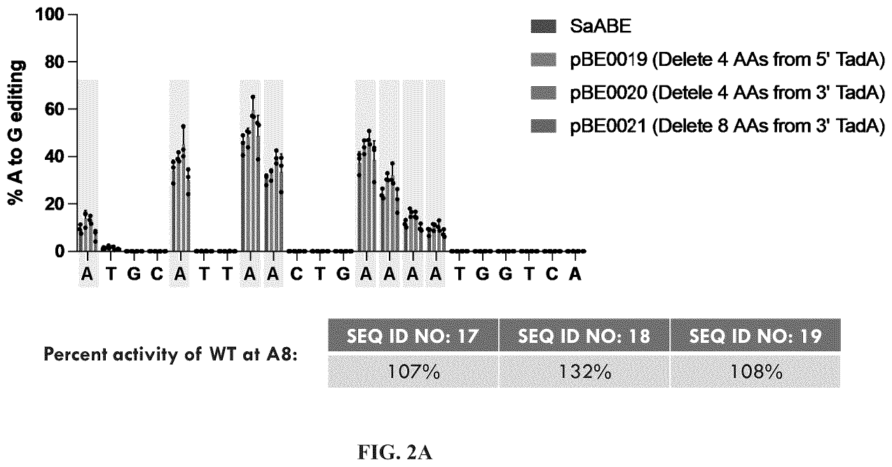

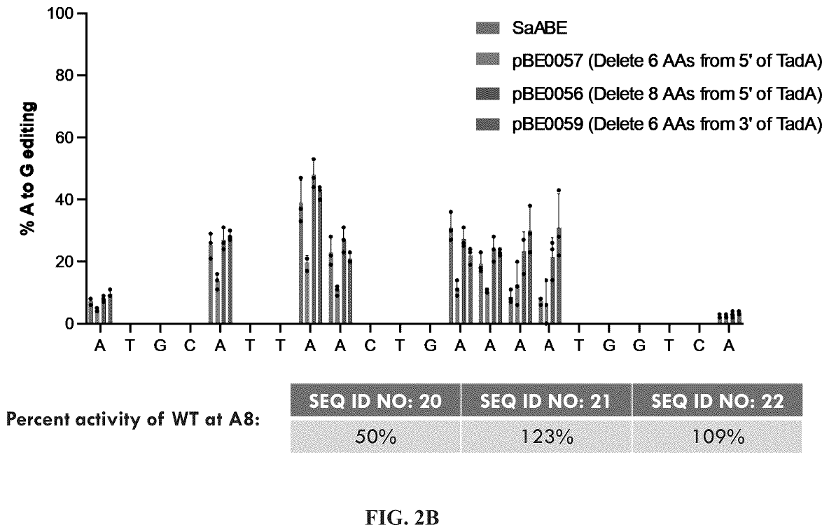

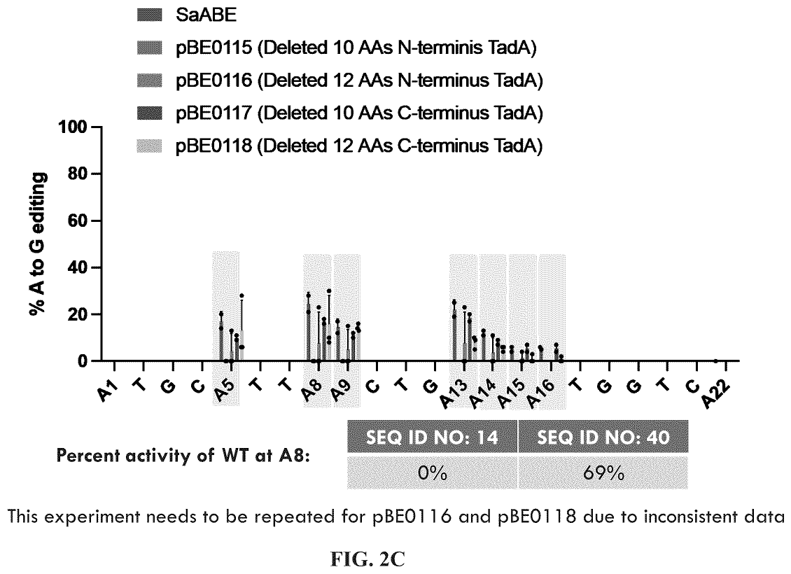

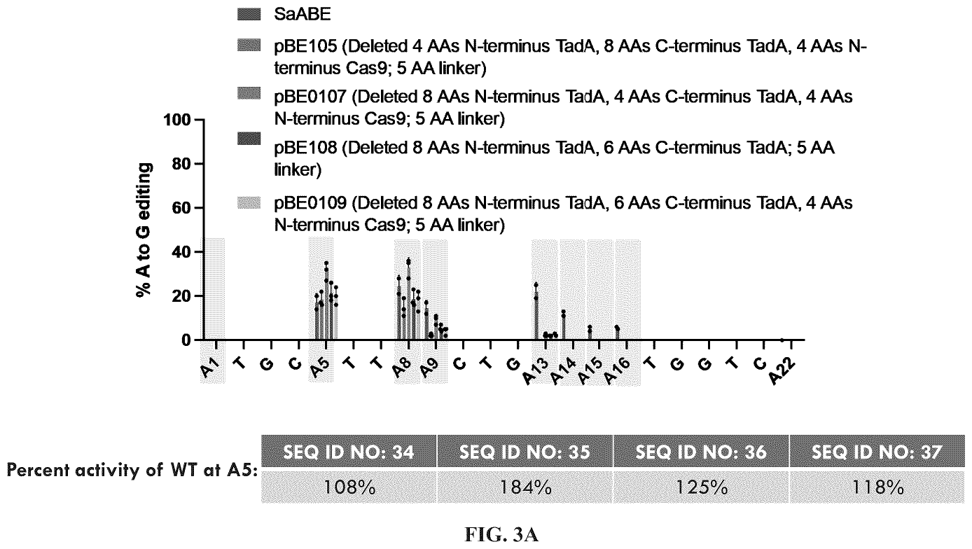

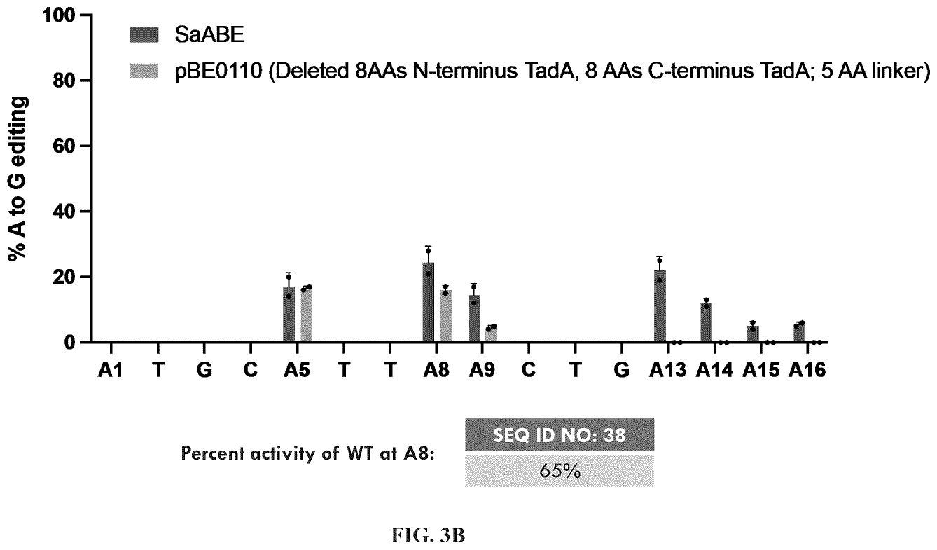

Next, the termini of TadA-8e were trimmed. Deletions of up to 8 amino acids on both termini of TadA-8e were well-tolerated as shown in FIGS. 2A, 2B, and 2C. The shortened termini of TadA-8e experiments were then combined with the shortened linkers of Example 1. Results are shown in FIGS. 3A and 3B where SEQ ID NO: 36 and SEQ ID NO: 38 had nucleobase activity of 125% and 65% of wild type nucleobase editor activity, respectively. The most efficient combination was removing 8 amino acids from the N-terminus of TadA-8e and 6 amino acids from the C-terminus of TadA8e. Such truncations can likely be utilized with other Cas9 variants.

### Example 3: Removal of Amino Acids from Cas9 Nuclease

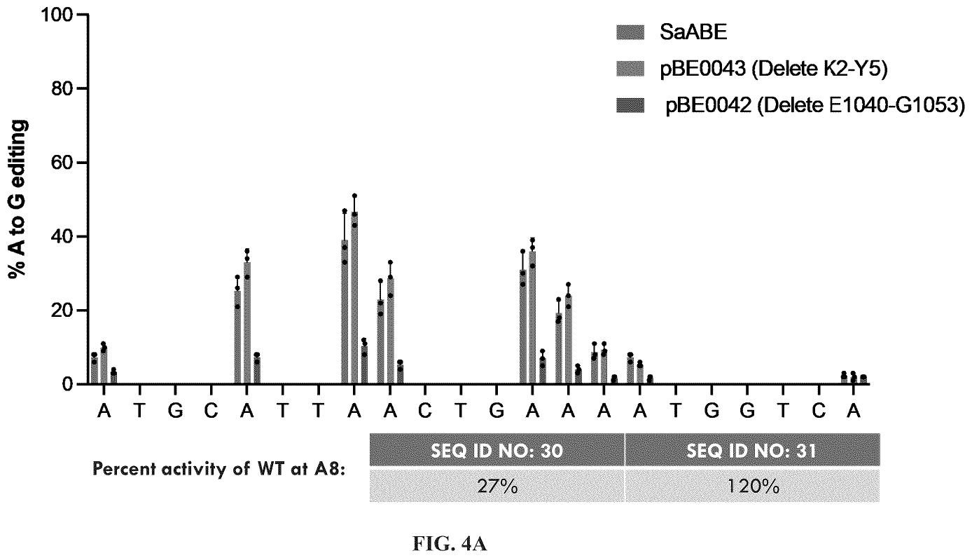

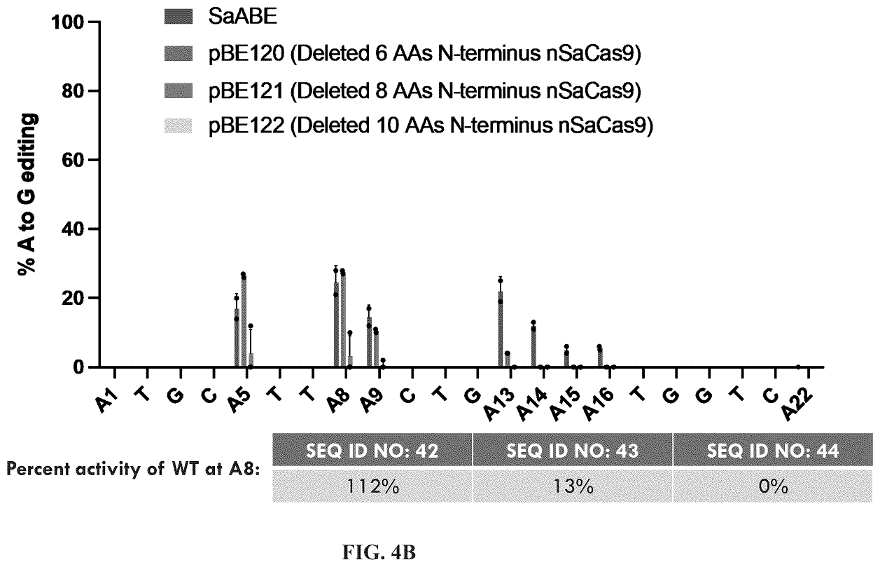

Amino acids from the termini of nSaCas9 were also trimmed (FIGS. 4A and 4B). Up to 4 amino acids could be trimmed from the N-terminus of nSaCa9. SaABE did not tolerate amino acids trimmed from the C-terminus of nSaCas9. SaABE editing levels were maintained when the TadA termini truncations, shortened linker, and nSaCas9 N-terminus truncation were combined.

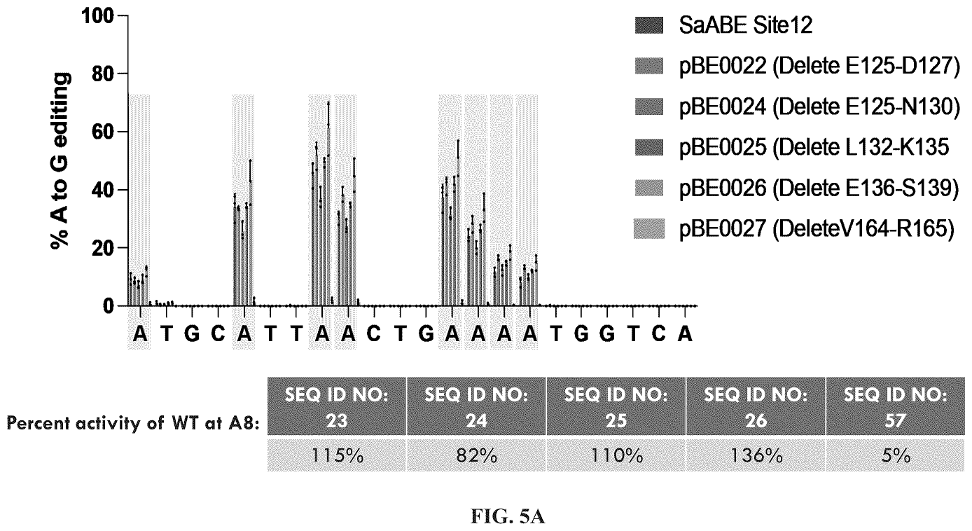

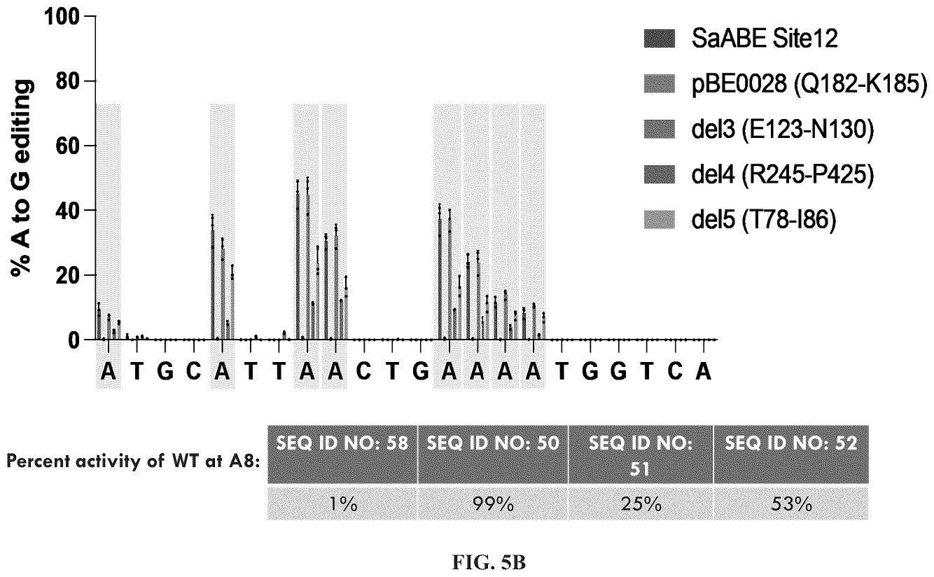

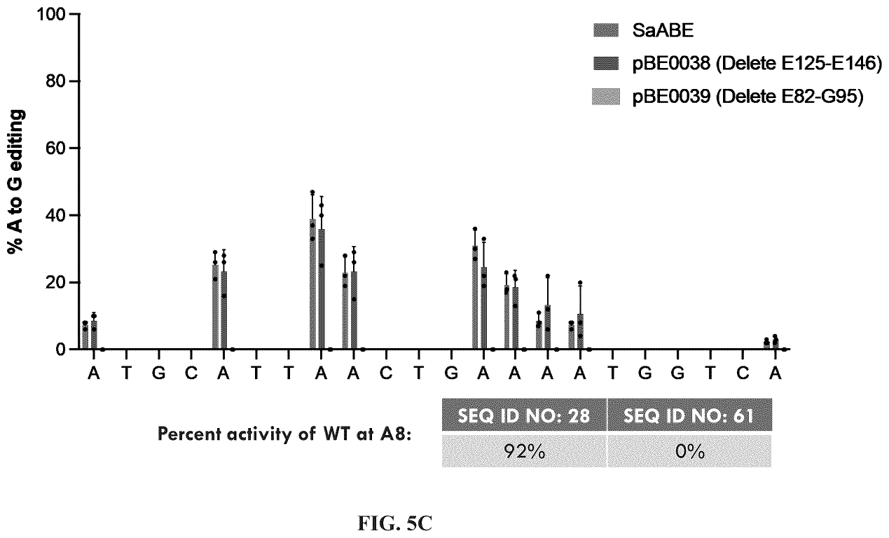

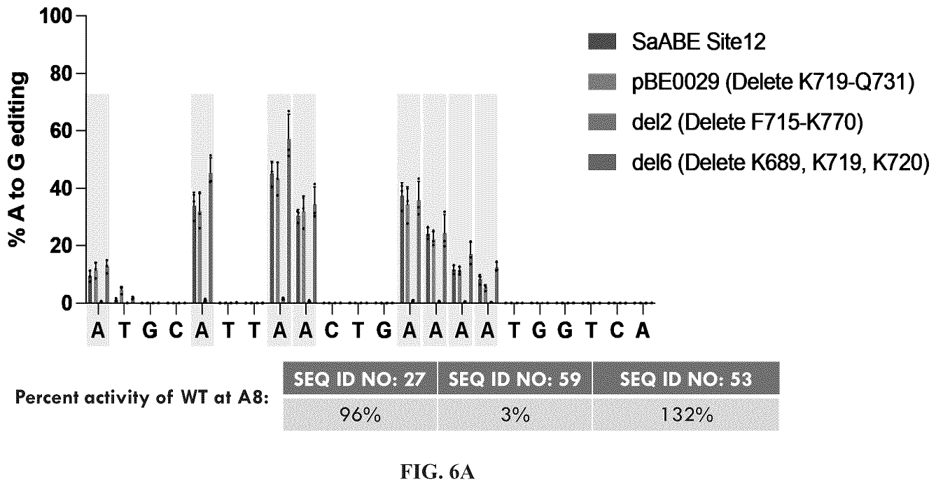

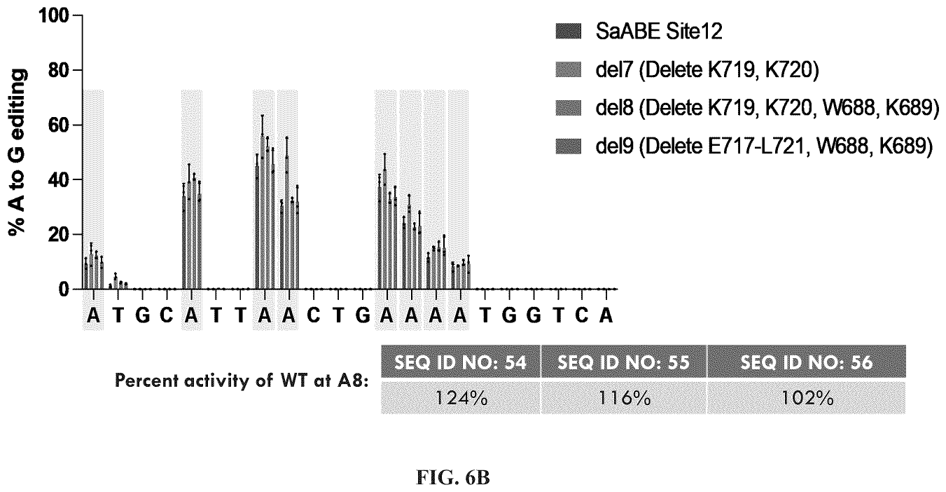

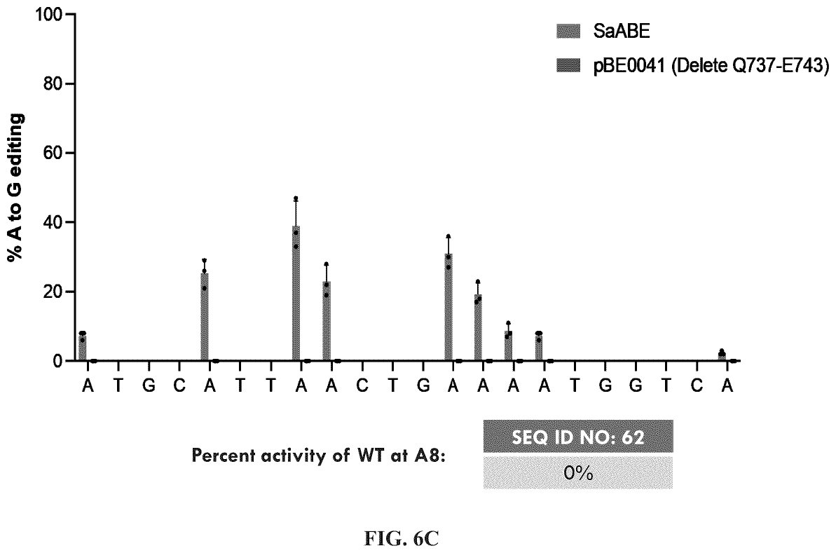

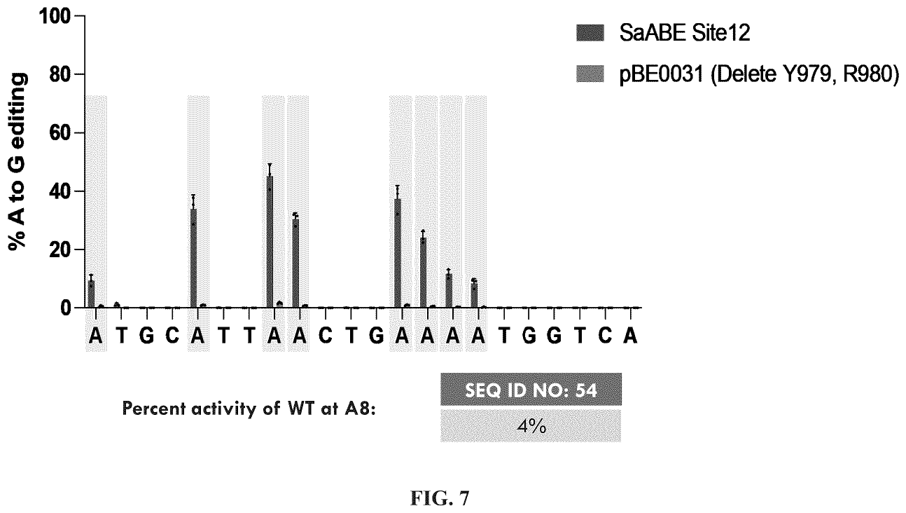

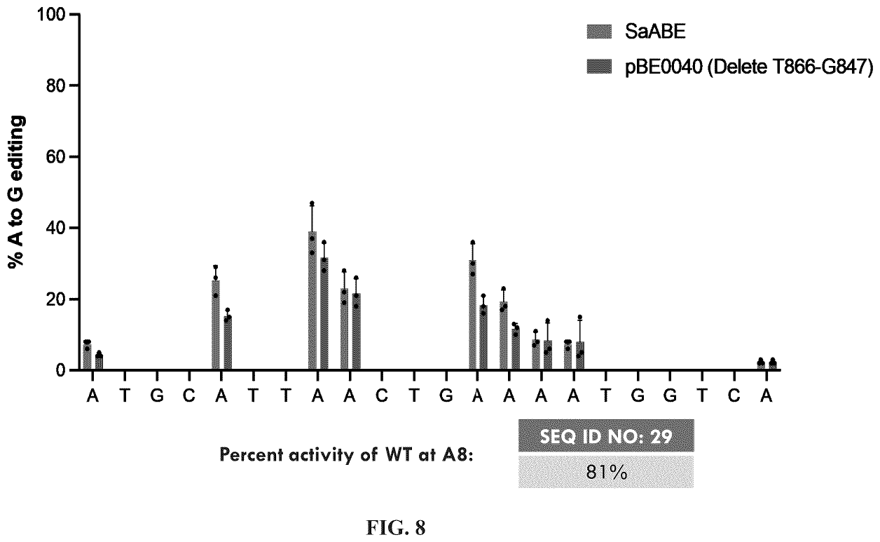

Stretches of amino acids within the REC-lobe, RuvC domain, and PAM-interacting domain of nSaCas9 were trimmed. Certain truncations in the REC-lobe and RuvC domain were tolerated, while truncations in the PAM-interacting domain were not tolerated for base editing activity. A stretch of up to 22 amino acids could be deleted in the REC-lobe domain as shown in FIGS. 5A, 5B, and 5C. This truncation was tolerated in combination with other truncations of Examples 1 and 2 and this combination of deletions brings the AAV genome size to 4.9 kb. Removing a stretch of up to 13 amino acids was tolerated in the RuvC domain (FIGS. 6A, 6B, and 6C). However, there was a large reduction in editing activity when deletions in the RuvC domain were combined with other deletions (in the REC-lobe and/or TadA-8e/linker).

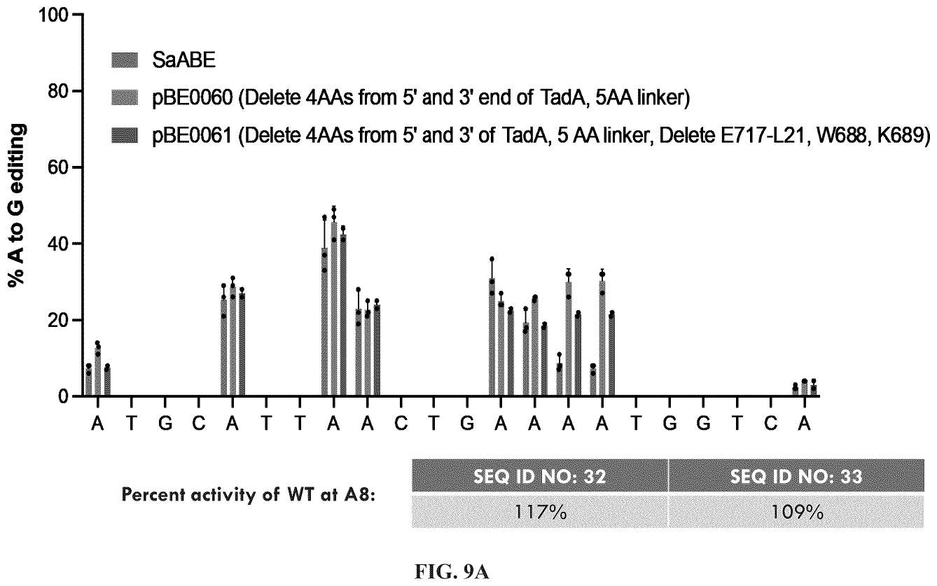

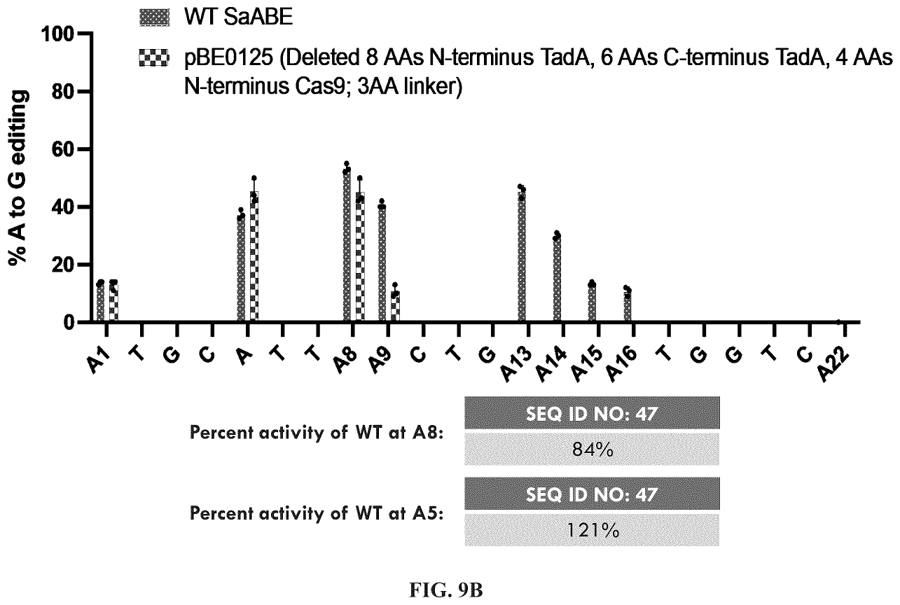

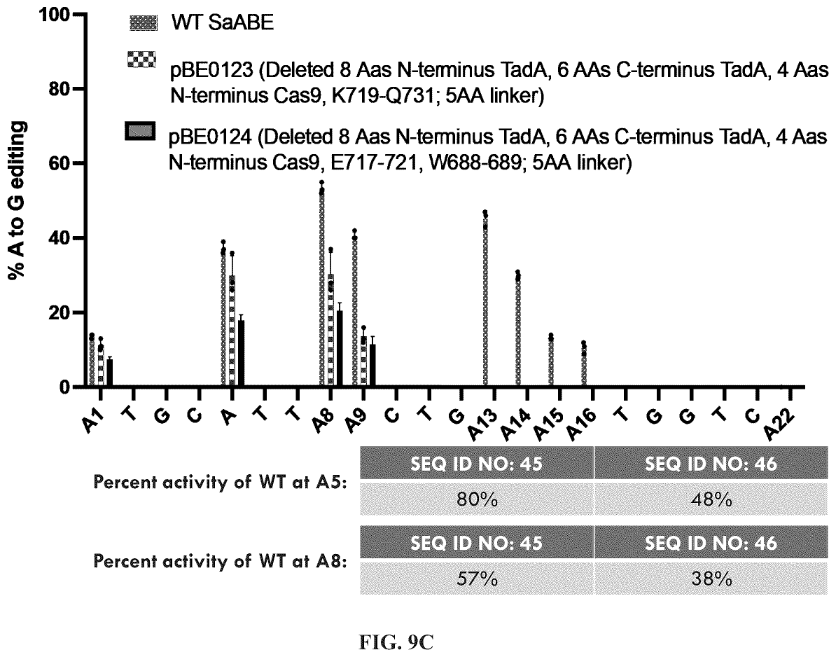

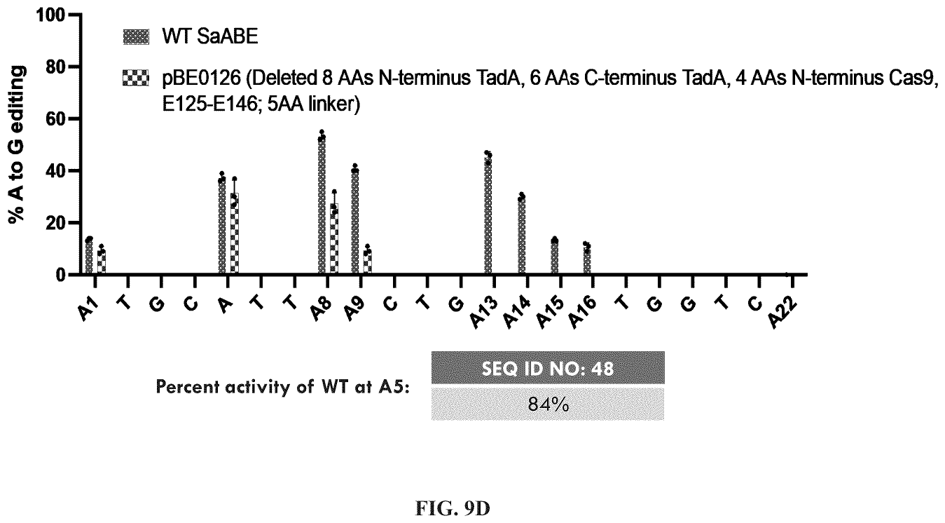

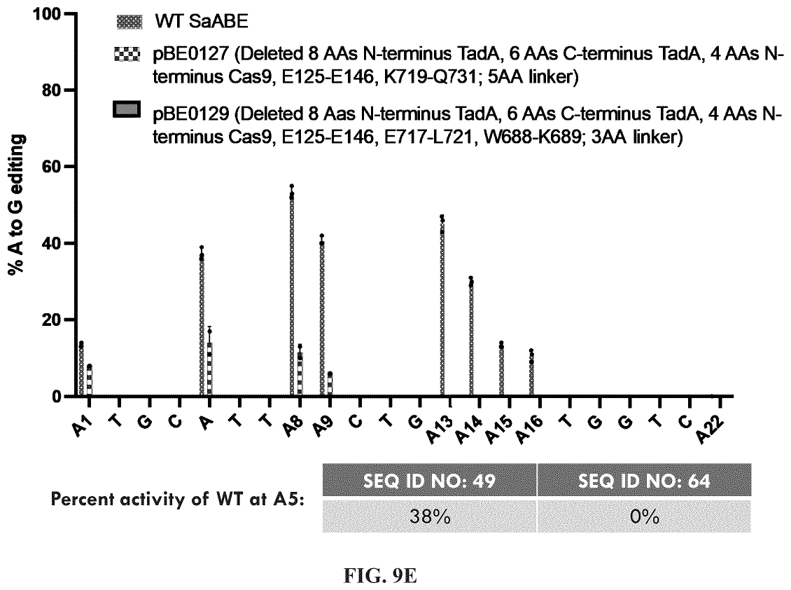

When combining selected deletions in TadA-8e, the linker, the N-terminus of nSaCas9, the REC-lobe and the RuvC domain, there were several modified BEs with activity over 100% of the activity of the wild type SaABE, SEQ ID NO: 32, SEQ ID NO: 33, SEQ ID NO: 47 (FIGS. 9A and 9B). The overall activity after combining deletions in TadA-8e, the linker, the N-terminus of nSaCas9, the REC-lobe and the RuvC domain is less than 50% the activity of the original SaABE. However, it may be possible to regain activity through an arginine scan to increase binding energy.

Base editor activity of all modified BEs compared to activity of a reference BE (SEQ ID NO: 3) is shown in Table 1. Table 1 provides the representative SEQ ID No of the modified BE and shows the sequences with modifications as compared to the reference BE. The sequences in Table 1 are presented to show a visual representation of the modifications in the sequences used where the TadA is presented as bolded text, the linker between the deaminase and the nuclease as italicized, and the nuclease as regular font.

TABLE 1

| | | | Activity |
|---|---|---|---|
| | | | compared |
| | Representative | | to |
| Name | SEQ ID NO: | Sequence | reference |
| pBE0001 | 3 | SEVEFSHEYWMRHALTLAKRARDEREVPVGAVLVLNNRVIGE | N/A |
| (Reference) | | GWNRAIGLHDPTAHAEIMALRQGGLVMQNYRLIDATLYVTFE | |
| | | PCVMCAGAMIHSRIGRVVFGVRNSKRGAAGSLMNVLNYPGMN | |
| | | HRVEITEGILADECAALLCDFYRMPRQVFNAQKKAQSSINSG | |
| | | GSSGGSSGSETPGTSESATPESSGGSSGGSKRNYILGLAIGI | |
| | | TSVGYGIIDYETRDVIDAGVRLFKEANVENNEGRRSKRGARR | |
| | | LKRRRRHRIQRVKKLLFDYNLLTDHSELSGINPYEARVKGLS | |
| | | QKLSEEEFSAALLHLAKRRGVHNVNEVEEDTGNELSTKEQIS | |
| | | RNSKALEEKYVAELQLERLKKDGEVRGSINRFKTSDYVKEAK | |
| | | QLLKVQKAYHQLDQSFIDTYIDLLETRRTYYEGPGEGSPFGW | |
| | | KDIKEWYEMLMGHCTYFPEELRSVKYAYNADLYNALNDLNNL | |
| | | VITRDENEKLEYYEKFQIIENVFKQKKKPTLKQIAKEILVNE | |
| | | EDIKGYRVTSTGKPEFTNLKVYHDIKDITARKEIIENAELLD | |
| | | QIAKILTIYQSSEDIQEELTNLNSELTQEEIEQISNLKGYTG | |
| | | THNLSLKAINLILDELWHTNDNQIAIFNRLKLVPKKVDLSQQ | |
| | | KEIPTTLVDDFILSPVVKRSFIQSIKVINAIIKKYGLPNDII | |
| | | IELAREKNSKDAQKMINEMQKRNRQTNERIEEIIRTTGKENA | |
| | | KYLIEKIKLHDMQEGKCLYSLEAIPLEDLLNNPFNYEVDHII | |
| | | PRSVSFDNSFNNKVLVKQEENSKKGNRTPFQYLSSSDSKISY | |
| | | ETFKKHILNLAKGKGRISKTKKEYLLEERDINRFSVQKDFIN | |
| | | RNLVDTRYATRGLMNLLRSYFRVNNLDVKVKSINGGFTSFLR | |
| | | RKWKFKKERNKGYKHHAEDALIIANADFIFKEWKKLDKAKKV | |
| | | MENQMFEEKQAESMPEIETEQEYKEIFITPHQIKHIKDFKDY | |
| | | KYSHRVDKKPNRELINDTLYSTRKDDKGNTLIVNNLNGLYDK | |
| | | DNDKLKKLINKSPEKLLMYHHDPQTYQKLKLIMEQYGDEKNP | |
| | | LYKYYEETGNYLTKYSKKDNGPVIKKIKYYGNKLNAHLDITD | |
| | | DYPNSRNKVVKLSLKPYRFDVYLDNGVYKFVTVKNLDVIKKE | |
| | | NYYEVNSKCYEEAKKLKKISNQAEFIASFYNNDLIKINGELY | |
| | | RVIGVNNDLLNRIEVNMIDITYREYLENMNDKRPPRIIKTIA | |
| | | SKTQSIKKYSTDILGNLYEVKSKKHPQIIKKG | |
| pBE0015 | 14 | SEVEFSHEYWMRHALTLAKRARDEREVPVGAVLVLNNRVIGE | >90% |
| | | GWNRAIGLHDPTAHAEIMALRQGGLVMQNYRLIDATLYVTFE | (A5) |
| | | PCVMCAGAMIHSRIGRVVFGVRNSKRGAAGSLMNVLNYPGMN | |
| | | HRVEITEGILADECAALLCDFYRMPRQVFNAQKKAQSSINPA | |
| | | PAPKRNYILGLAIGITSVGYGIIDYETRDVIDAGVRLFKEAN | |
| | | VENNEGRRSKRGARRLKRRRRHRIQRVKKLLFDYNLLTDHSE | |
| | | LSGINPYEARVKGLSQKLSEEEFSAALLHLAKRRGVHNVNEV | |
| | | EEDTGNELSTKEQISRNSKALEEKYVAELQLERLKKDGEVRG | |
| | | SINRFKTSDYVKEAKQLLKVQKAYHQLDQSFIDTYIDLLETR | |
| | | RTYYEGPGEGSPFGWKDIKEWYEMLMGHCTYFPEELRSVKYA | |
| | | YNADLYNALNDLNNLVITRDENEKLEYYEKFQIIENVFKQKK | |
| | | KPTLKQIAKEILVNEEDIKGYRVTSTGKPEFTNLKVYHDIKD | |
| | | ITARKEIIENAELLDQIAKILTIYQSSEDIQEELTNLNSELT | |
| | | QEEIEQISNLKGYTGTHNLSLKAINLILDELWHTNDNQIAIF | |
| | | NRLKLVPKKVDLSQQKEIPTTLVDDFILSPVVKRSFIQSIKV | |
| | | INAIIKKYGLPNDIIIELAREKNSKDAQKMINEMQKRNRQTN | |
| | | ERIEEIIRTTGKENAKYLIEKIKLHDMQEGKCLYSLEAIPLE | |
| | | DLLNNPFNYEVDHIIPRSVSFDNSFNNKVLVKQEENSKKGNR | |
| | | TPFQYLSSSDSKISYETFKKHILNLAKGKGRISKTKKEYLLE | |
| | | ERDINRFSVQKDFINRNLVDTRYATRGLMNLLRSYFRVNNLD | |
| | | VKVKSINGGFTSFLRRKWKFKKERNKGYKHHAEDALIIANAD | |
| | | FIFKEWKKLDKAKKVMENQMFEEKQAESMPEIETEQEYKEIF | |
| | | ITPHQIKHIKDFKDYKYSHRVDKKPNRELINDTLYSTRKDDK | |
| | | GNTLIVNNLNGLYDKDNDKLKKLINKSPEKLLMYHHDPQTYQ | |
| | | KLKLIMEQYGDEKNPLYKYYEETGNYLTKYSKKDNGPVIKKI | |
| | | KYYGNKLNAHLDITDDYPNSRNKVVKLSLKPYRFDVYLDNGV | |
| | | YKFVTVKNLDVIKKENYYEVNSKCYEEAKKLKKISNQAEFIA | |
| | | SFYNNDLIKINGELYRVIGVNNDLLNRIEVNMIDITYREYLE | |
| | | NMNDKRPPRIIKTIASKTQSIKKYSTDILGNLYEVKSKKHPQ | |
| | | IIKKG | |
| pBE0016 | 15 | SEVEFSHEYWMRHALTLAKRARDEREVPVGAVLVLNNRVIGE | >90% |
| | | GWNRAIGLHDPTAHAEIMALRQGGLVMQNYRLIDATLYVTFE | (A5) |
| | | PCVMCAGAMIHSRIGRVVFGVRNSKRGAAGSLMNVLNYPGMN | |
| | | HRVEITEGILADECAALLCDFYRMPRQVFNAQKKAQSSINPA | |
| | | PAPAPKRNYILGLAIGITSVGYGIIDYETRDVIDAGVRLFKE | |
| | | ANVENNEGRRSKRGARRLKRRRRHRIQRVKKLLFDYNLLTDH | |
| | | SELSGINPYEARVKGLSQKLSEEEFSAALLHLAKRRGVHNVN | |
| | | EVEEDTGNELSTKEQISRNSKALEEKYVAELQLERLKKDGEV | |
| | | RGSINRFKTSDYVKEAKQLLKVQKAYHQLDQSFIDTYIDLLE | |
| | | TRRTYYEGPGEGSPFGWKDIKEWYEMLMGHCTYFPEELRSVK | |
| | | YAYNADLYNALNDLNNLVITRDENEKLEYYEKFQIIENVEKQ | |
| | | KKKPTLKQIAKEILVNEEDIKGYRVTSTGKPEFTNLKVYHDI | |
| | | KDITARKEIIENAELLDQIAKILTIYQSSEDIQEELTNLNSE | |
| | | LTQEEIEQISNLKGYTGTHNLSLKAINLILDELWHTNDNQIA | |
| | | IFNRLKLVPKKVDLSQQKEIPTTLVDDFILSPVVKRSFIQSI | |
| | | KVINAIIKKYGLPNDIIIELAREKNSKDAQKMINEMQKRNRQ | |
| | | TNERIEEIIRTTGKENAKYLIEKIKLHDMQEGKCLYSLEAIP | |
| | | LEDLLNNPFNYEVDHIIPRSVSFDNSFNNKVLVKQEENSKKG | |
| | | NRTPFQYLSSSDSKISYETFKKHILNLAKGKGRISKTKKEYL | |
| | | LEERDINRFSVQKDFINRNLVDTRYATRGLMNLLRSYFRVNN | |
| | | LDVKVKSINGGFTSFLRRKWKFKKERNKGYKHHAEDALIIAN | |
| | | ADFIFKEWKKLDKAKKVMENQMFEEKQAESMPEIETEQEYKE | |
| | | IFITPHQIKHIKDFKDYKYSHRVDKKPNRELINDTLYSTRKD | |
| | | DKGNTLIVNNLNGLYDKDNDKLKKLINKSPEKLLMYHHDPQT | |
| | | YQKLKLIMEQYGDEKNPLYKYYEETGNYLTKYSKKDNGPVIK | |
| | | KIKYYGNKLNAHLDITDDYPNSRNKVVKLSLKPYRFDVYLDN | |
| | | GVYKFVTVKNLDVIKKENYYEVNSKCYEEAKKLKKISNQAEF | |
| | | IASFYNNDLIKINGELYRVIGVNNDLLNRIEVNMIDITYREY | |
| | | LENMNDKRPPRIIKTIASKTQSIKKYSTDILGNLYEVKSKKH | |
| | | PQIIKKG | |
| pBE0119 | 16 | SEVEFSHEYWMRHALTLAKRARDEREVPVGAVLVLNNRVIGE | >90% |
| | | GWNRAIGLHDPTAHAEIMALRQGGLVMQNYRLIDATLYVTFE | (A8) |
| | | PCVMCAGAMIHSRIGRVVFGVRNSKRGAAGSLMNVLNYPGMN | |
| | | HRVEITEGILADECAALLCDFYRMPRQVFNAQKKAQSSINPA | |
| | | PKRNYILGLAIGITSVGYGIIDYETRDVIDAGVRLFKEANVE | |
| | | NNEGRRSKRGARRLKRRRRHRIQRVKKLLFDYNLLTDHSELS | |
| | | GINPYEARVKGLSQKLSEEEFSAALLHLAKRRGVHNVNEVEE | |
| | | DTGNELSTKEQISRNSKALEEKYVAELQLERLKKDGEVRGSI | |
| | | NRFKTSDYVKEAKQLLKVQKAYHQLDQSFIDTYIDLLETRRT | |
| | | YYEGPGEGSPFGWKDIKEWYEMLMGHCTYFPEELRSVKYAYN | |
| | | ADLYNALNDLNNLVITRDENEKLEYYEKFQIIENVFKQKKKP | |
| | | TLKQIAKEILVNEEDIKGYRVTSTGKPEFTNLKVYHDIKDIT | |
| | | ARKEIIENAELLDQIAKILTIYQSSEDIQEELTNLNSELTQE | |
| | | EIEQISNLKGYTGTHNLSLKAINLILDELWHTNDNQIAIFNR | |
| | | LKLVPKKVDLSQQKEIPTTLVDDFILSPVVKRSFIQSIKVIN | |
| | | AIIKKYGLPNDIIIELAREKNSKDAQKMINEMQKRNRQTNER | |
| | | IEEIIRTTGKENAKYLIEKIKLHDMQEGKCLYSLEAIPLEDL | |
| | | LNNPFNYEVDHIIPRSVSFDNSFNNKVLVKQEENSKKGNRTP | |
| | | FQYLSSSDSKISYETFKKHILNLAKGKGRISKTKKEYLLEER | |
| | | DINRFSVQKDFINRNLVDTRYATRGLMNLLRSYFRVNNLDVK | |
| | | VKSINGGFTSFLRRKWKFKKERNKGYKHHAEDALIIANADFI | |
| | | FKEWKKLDKAKKVMENQMFEEKQAESMPEIETEQEYKEIFIT | |
| | | PHQIKHIKDFKDYKYSHRVDKKPNRELINDTLYSTRKDDKGN | |
| | | TLIVNNLNGLYDKDNDKLKKLINKSPEKLLMYHHDPQTYQKL | |
| | | KLIMEQYGDEKNPLYKYYEETGNYLTKYSKKDNGPVIKKIKY | |
| | | YGNKLNAHLDITDDYPNSRNKVVKLSLKPYRFDVYLDNGVYK | |
| | | FVTVKNLDVIKKENYYEVNSKCYEEAKKLKKISNQAEFIASF | |
| | | YNNDLIKINGELYRVIGVNNDLLNRIEVNMIDITYREYLENM | |
| | | NDKRPPRIIKTIASKTQSIKKYSTDILGNLYEVKSKKHPQII | |
| | | KKG | |
| pBE0019 | 17 | FSHEYWMRHALTLAKRARDEREVPVGAVLVLNNRVIGEGWNR | >90% |
| | | AIGLHDPTAHAEIMALRQGGLVMQNYRLIDATLYVTFEPCVM | (A8) |
| | | CAGAMIHSRIGRVVFGVRNSKRGAAGSLMNVLNYPGMNHRVE | |
| | | ITEGILADECAALLCDFYRMPRQVFNAQKKAQSSINSGGSSG | |
| | | GSSGSETPGTSESATPESSGGSSGGSKRNYILGLAIGITSVG | |
| | | YGIIDYETRDVIDAGVRLFKEANVENNEGRRSKRGARRLKRR | |
| | | RRHRIQRVKKLLFDYNLLTDHSELSGINPYEARVKGLSQKLS | |
| | | EEEFSAALLHLAKRRGVHNVNEVEEDTGNELSTKEQISRNSK | |
| | | ALEEKYVAELQLERLKKDGEVRGSINRFKTSDYVKEAKQLLK | |
| | | VQKAYHQLDQSFIDTYIDLLETRRTYYEGPGEGSPFGWKDIK | |
| | | EWYEMLMGHCTYFPEELRSVKYAYNADLYNALNDLNNLVITR | |
| | | DENEKLEYYEKFQIIENVFKQKKKPTLKQIAKEILVNEEDIK | |
| | | GYRVTSTGKPEFTNLKVYHDIKDITARKEIIENAELLDQIAK | |
| | | ILTIYQSSEDIQEELTNLNSELTQEEIEQISNLKGYTGTHNL | |
| | | SLKAINLILDELWHTNDNQIAIFNRLKLVPKKVDLSQQKEIP | |
| | | TTLVDDFILSPVVKRSFIQSIKVINAIIKKYGLPNDIIIELA | |
| | | REKNSKDAQKMINEMQKRNRQTNERIEEIIRTTGKENAKYLI | |
| | | EKIKLHDMQEGKCLYSLEAIPLEDLLNNPFNYEVDHIIPRSV | |
| | | SFDNSFNNKVLVKQEENSKKGNRTPFQYLSSSDSKISYETFK | |
| | | KHILNLAKGKGRISKTKKEYLLEERDINRFSVQKDFINRNLV | |
| | | DTRYATRGLMNLLRSYFRVNNLDVKVKSINGGFTSFLRRKWK | |
| | | FKKERNKGYKHHAEDALIIANADFIFKEWKKLDKAKKVMENQ | |
| | | MFEEKQAESMPEIETEQEYKEIFITPHQIKHIKDFKDYKYSH | |
| | | RVDKKPNRELINDTLYSTRKDDKGNTLIVNNLNGLYDKDNDK | |
| | | LKKLINKSPEKLLMYHHDPQTYQKLKLIMEQYGDEKNPLYKY | |
| | | YEETGNYLTKYSKKDNGPVIKKIKYYGNKLNAHLDITDDYPN | |
| | | SRNKVVKLSLKPYRFDVYLDNGVYKFVTVKNLDVIKKENYYE | |
| | | VNSKCYEEAKKLKKISNQAEFIASFYNNDLIKINGELYRVIG | |
| | | VNNDLLNRIEVNMIDITYREYLENMNDKRPPRIIKTIASKTQ | |
| | | SIKKYSTDILGNLYEVKSKKHPQIIKKG | |
| pBE0020 | 18 | SEVEFSHEYWMRHALTLAKRARDEREVPVGAVLVLNNRVIGE | >90% |
| | | GWNRAIGLHDPTAHAEIMALRQGGLVMQNYRLIDATLYVTFE | (A8) |
| | | PCVMCAGAMIHSRIGRVVFGVRNSKRGAAGSLMNVLNYPGMN | |
| | | HRVEITEGILADECAALLCDFYRMPRQVFNAQKKAQSGGSSG | |
| | | GSSGSETPGTSESATPESSGGSSGGSKRNYILGLAIGITSVG | |
| | | YGIIDYETRDVIDAGVRLFKEANVENNEGRRSKRGARRLKRR | |
| | | RRHRIQRVKKLLFDYNLLTDHSELSGINPYEARVKGLSQKLS | |
| | | EEEFSAALLHLAKRRGVHNVNEVEEDTGNELSTKEQISRNSK | |
| | | ALEEKYVAELQLERLKKDGEVRGSINRFKTSDYVKEAKQLLK | |
| | | VQKAYHQLDQSFIDTYIDLLETRRTYYEGPGEGSPFGWKDIK | |
| | | EWYEMLMGHCTYFPEELRSVKYAYNADLYNALNDLNNLVITR | |
| | | DENEKLEYYEKFQIIENVFKQKKKPTLKQIAKEILVNEEDIK | |
| | | GYRVTSTGKPEFTNLKVYHDIKDITARKEIIENAELLDQIAK | |
| | | ILTIYQSSEDIQEELTNLNSELTQEEIEQISNLKGYTGTHNL | |
| | | SLKAINLILDELWHTNDNQIAIFNRLKLVPKKVDLSQQKEIP | |
| | | TTLVDDFILSPVVKRSFIQSIKVINAIIKKYGLPNDIIIELA | |
| | | REKNSKDAQKMINEMQKRNRQTNERIEEIIRTTGKENAKYLI | |
| | | EKIKLHDMQEGKCLYSLEAIPLEDLLNNPFNYEVDHIIPRSV | |
| | | SFDNSFNNKVLVKQEENSKKGNRTPFQYLSSSDSKISYETFK | |
| | | KHILNLAKGKGRISKTKKEYLLEERDINRFSVQKDFINRNLV | |
| | | DTRYATRGLMNLLRSYFRVNNLDVKVKSINGGFTSFLRRKWK | |
| | | FKKERNKGYKHHAEDALIIANADFIFKEWKKLDKAKKVMENQ | |
| | | MFEEKQAESMPEIETEQEYKEIFITPHQIKHIKDFKDYKYSH | |
| | | RVDKKPNRELINDTLYSTRKDDKGNTLIVNNLNGLYDKDNDK | |
| | | LKKLINKSPEKLLMYHHDPQTYQKLKLIMEQYGDEKNPLYKY | |
| | | YEETGNYLTKYSKKDNGPVIKKIKYYGNKLNAHLDITDDYPN | |
| | | SRNKVVKLSLKPYRFDVYLDNGVYKFVTVKNLDVIKKENYYE | |
| | | VNSKCYEEAKKLKKISNQAEFIASFYNNDLIKINGELYRVIG | |
| | | VNNDLLNRIEVNMIDITYREYLENMNDKRPPRIIKTIASKTQ | |
| | | SIKKYSTDILGNLYEVKSKKHPQIIKKG | |
| pBE0021 | 19 | SEVEFSHEYWMRHALTLAKRARDEREVPVGAVLVLNNRVIGE | >90% |
| | | GWNRAIGLHDPTAHAEIMALRQGGLVMQNYRLIDATLYVTFE | (A8) |
| | | PCVMCAGAMIHSRIGRVVFGVRNSKRGAAGSLMNVLNYPGMN | |
| | | HRVEITEGILADECAALLCDFYRMPRQVFNAQSGGSSGGSSG | |
| | | SETPGTSESATPESSGGSSGGSKRNYILGLAIGITSVGYGII | |
| | | DYETRDVIDAGVRLFKEANVENNEGRRSKRGARRLKRRRRHR | |
| | | IQRVKKLLFDYNLLTDHSELSGINPYEARVKGLSQKLSEEEF | |
| | | SAALLHLAKRRGVHNVNEVEEDTGNELSTKEQISRNSKALEE | |
| | | KYVAELQLERLKKDGEVRGSINRFKTSDYVKEAKQLLKVQKA | |
| | | YHQLDQSFIDTYIDLLETRRTYYEGPGEGSPFGWKDIKEWYE | |
| | | MLMGHCTYFPEELRSVKYAYNADLYNALNDLNNLVITRDENE | |
| | | KLEYYEKFQIIENVFKQKKKPTLKQIAKEILVNEEDIKGYRV | |
| | | TSTGKPEFTNLKVYHDIKDITARKEIIENAELLDQIAKILTI | |
| | | YQSSEDIQEELTNLNSELTQEEIEQISNLKGYTGTHNLSLKA | |
| | | INLILDELWHTNDNQIAIFNRLKLVPKKVDLSQQKEIPTTLV | |
| | | DDFILSPVVKRSFIQSIKVINAIIKKYGLPNDIIIELAREKN | |
| | | SKDAQKMINEMQKRNRQTNERIEEIIRTTGKENAKYLIEKIK | |
| | | LHDMQEGKCLYSLEAIPLEDLLNNPFNYEVDHIIPRSVSFDN | |
| | | SFNNKVLVKQEENSKKGNRTPFQYLSSSDSKISYETFKKHIL | |
| | | NLAKGKGRISKTKKEYLLEERDINRFSVQKDFINRNLVDTRY | |
| | | ATRGLMNLLRSYFRVNNLDVKVKSINGGFTSFLRRKWKFKKE | |
| | | RNKGYKHHAEDALIIANADFIFKEWKKLDKAKKVMENQMFEE | |
| | | KQAESMPEIETEQEYKEIFITPHQIKHIKDFKDYKYSHRVDK | |
| | | KPNRELINDTLYSTRKDDKGNTLIVNNLNGLYDKDNDKLKKL | |
| | | INKSPEKLLMYHHDPQTYQKLKLIMEQYGDEKNPLYKYYEET | |
| | | GNYLTKYSKKDNGPVIKKIKYYGNKLNAHLDITDDYPNSRNK | |
| | | VVKLSLKPYRFDVYLDNGVYKFVTVKNLDVIKKENYYEVNSK | |
| | | CYEEAKKLKKISNQAEFIASFYNNDLIKINGELYRVIGVNND | |
| | | LLNRIEVNMIDITYREYLENMNDKRPPRIIKTIASKTQSIKK | |
| | | YSTDILGNLYEVKSKKHPQIIKKG | |
| pBE0057 | 20 | HEYWMRHALTLAKRARDEREVPVGAVLVLNNRVIGEGWNRAI | 50-75% |
| | | GLHDPTAHAEIMALRQGGLVMQNYRLIDATLYVTFEPCVMCA | (A8) |
| | | GAMIHSRIGRVVFGVRNSKRGAAGSLMNVLNYPGMNHRVEIT | |
| | | EGILADECAALLCDFYRMPRQVFNAQKKAQSSINSGGSSGGS | |
| | | SGSETPGTSESATPESSGGSSGGSKRNYILGLAIGITSVGYG | |
| | | IIDYETRDVIDAGVRLFKEANVENNEGRRSKRGARRLKRRRR | |
| | | HRIQRVKKLLFDYNLLTDHSELSGINPYEARVKGLSQKLSEE | |
| | | EFSAALLHLAKRRGVHNVNEVEEDTGNELSTKEQISRNSKAL | |
| | | EEKYVAELQLERLKKDGEVRGSINRFKTSDYVKEAKQLLKVQ | |
| | | KAYHQLDQSFIDTYIDLLETRRTYYEGPGEGSPFGWKDIKEW | |
| | | YEMLMGHCTYFPEELRSVKYAYNADLYNALNDLNNLVITRDE | |
| | | NEKLEYYEKFQIIENVFKQKKKPTLKQIAKEILVNEEDIKGY | |
| | | RVTSTGKPEFTNLKVYHDIKDITARKEIIENAELLDQIAKIL | |
| | | TIYQSSEDIQEELTNLNSELTQEEIEQISNLKGYTGTHNLSL | |
| | | KAINLILDELWHTNDNQIAIFNRLKLVPKKVDLSQQKEIPTT | |
| | | LVDDFILSPVVKRSFIQSIKVINAIIKKYGLPNDIIIELARE | |
| | | KNSKDAQKMINEMQKRNRQTNERIEEIIRTTGKENAKYLIEK | |
| | | IKLHDMQEGKCLYSLEAIPLEDLLNNPFNYEVDHIIPRSVSF | |
| | | DNSFNNKVLVKQEENSKKGNRTPFQYLSSSDSKISYETFKKH | |
| | | ILNLAKGKGRISKTKKEYLLEERDINRFSVQKDFINRNLVDT | |
| | | RYATRGLMNLLRSYFRVNNLDVKVKSINGGFTSFLRRKWKFK | |
| | | KERNKGYKHHAEDALIIANADFIFKEWKKLDKAKKVMENQMF | |
| | | EEKQAESMPEIETEQEYKEIFITPHQIKHIKDFKDYKYSHRV | |
| | | DKKPNRELINDTLYSTRKDDKGNTLIVNNLNGLYDKDNDKLK | |
| | | KLINKSPEKLLMYHHDPQTYQKLKLIMEQYGDEKNPLYKYYE | |
| | | ETGNYLTKYSKKDNGPVIKKIKYYGNKLNAHLDITDDYPNSR | |
| | | NKVVKLSLKPYRFDVYLDNGVYKFVTVKNLDVIKKENYYEVN | |
| | | SKCYEEAKKLKKISNQAEFIASFYNNDLIKINGELYRVIGVN | |
| | | NDLLNRIEVNMIDITYREYLENMNDKRPPRIIKTIASKTQSI | |
| | | KKYSTDILGNLYEVKSKKHPQIIKKG | |
| pBE0058 | 21 | YWMRHALTLAKRARDEREVPVGAVLVLNNRVIGEGWNRAIGL | >90% |
| | | HDPTAHAEIMALRQGGLVMQNYRLIDATLYVTFEPCVMCAGA | (A8) |
| | | MIHSRIGRVVFGVRNSKRGAAGSLMNVLNYPGMNHRVEITEG | |
| | | ILADECAALLCDFYRMPRQVFNAQKKAQSSINSGGSSGGSSG | |
| | | SETPGTSESATPESSGGSSGGSKRNYILGLAIGITSVGYGII | |
| | | DYETRDVIDAGVRLFKEANVENNEGRRSKRGARRLKRRRRHR | |
| | | IQRVKKLLFDYNLLTDHSELSGINPYEARVKGLSQKLSEEEF | |
| | | SAALLHLAKRRGVHNVNEVEEDTGNELSTKEQISRNSKALEE | |
| | | KYVAELQLERLKKDGEVRGSINRFKTSDYVKEAKQLLKVQKA | |
| | | YHQLDQSFIDTYIDLLETRRTYYEGPGEGSPFGWKDIKEWYE | |
| | | MLMGHCTYFPEELRSVKYAYNADLYNALNDLNNLVITRDENE | |
| | | KLEYYEKFQIIENVFKQKKKPTLKQIAKEILVNEEDIKGYRV | |
| | | TSTGKPEFTNLKVYHDIKDITARKEIIENAELLDQIAKILTI | |
| | | YQSSEDIQEELTNLNSELTQEEIEQISNLKGYTGTHNLSLKA | |
| | | INLILDELWHTNDNQIAIFNRLKLVPKKVDLSQQKEIPTTLV | |
| | | DDFILSPVVKRSFIQSIKVINAIIKKYGLPNDIIIELAREKN | |
| | | SKDAQKMINEMQKRNRQTNERIEEIIRTTGKENAKYLIEKIK | |
| | | LHDMQEGKCLYSLEAIPLEDLLNNPFNYEVDHIIPRSVSFDN | |
| | | SFNNKVLVKQEENSKKGNRTPFQYLSSSDSKISYETFKKHIL | |
| | | NLAKGKGRISKTKKEYLLEERDINRESVQKDFINRNLVDTRY | |
| | | ATRGLMNLLRSYFRVNNLDVKVKSINGGFTSFLRRKWKFKKE | |
| | | RNKGYKHHAEDALIIANADFIFKEWKKLDKAKKVMENQMFEE | |
| | | KQAESMPEIETEQEYKEIFITPHQIKHIKDFKDYKYSHRVDK | |
| | | KPNRELINDTLYSTRKDDKGNTLIVNNLNGLYDKDNDKLKKL | |
| | | INKSPEKLLMYHHDPQTYQKLKLIMEQYGDEKNPLYKYYEET | |
| | | GNYLTKYSKKDNGPVIKKIKYYGNKLNAHLDITDDYPNSRNK | |
| | | VVKLSLKPYRFDVYLDNGVYKFVTVKNLDVIKKENYYEVNSK | |
| | | CYEEAKKLKKISNQAEFIASFYNNDLIKINGELYRVIGVNND | |
| | | LLNRIEVNMIDITYREYLENMNDKRPPRIIKTIASKTQSIKK | |
| | | YSTDILGNLYEVKSKKHPQIIKKG | |
| pBE0059 | 22 | SEVEFSHEYWMRHALTLAKRARDEREVPVGAVLVLNNRVIGE | >90% |
| | | GWNRAIGLHDPTAHAEIMALRQGGLVMQNYRLIDATLYVTFE | (A8) |
| | | PCVMCAGAMIHSRIGRVVFGVRNSKRGAAGSLMNVLNYPGMN | |
| | | HRVEITEGILADECAALLCDFYRMPRQVFNAQKKSGGSSGGS | |
| | | SGSETPGTSESATPESSGGSSGGSKRNYILGLAIGITSVGYG | |
| | | IIDYETRDVIDAGVRLFKEANVENNEGRRSKRGARRLKRRRR | |
| | | HRIQRVKKLLFDYNLLTDHSELSGINPYEARVKGLSQKLSEE | |
| | | EFSAALLHLAKRRGVHNVNEVEEDTGNELSTKEQISRNSKAL | |
| | | EEKYVAELQLERLKKDGEVRGSINRFKTSDYVKEAKQLLKVQ | |
| | | KAYHQLDQSFIDTYIDLLETRRTYYEGPGEGSPFGWKDIKEW | |
| | | YEMLMGHCTYFPEELRSVKYAYNADLYNALNDLNNLVITRDE | |
| | | NEKLEYYEKFQIIENVFKQKKKPTLKQIAKEILVNEEDIKGY | |
| | | RVTSTGKPEFTNLKVYHDIKDITARKEIIENAELLDQIAKIL | |
| | | TIYQSSEDIQEELTNLNSELTQEEIEQISNLKGYTGTHNLSL | |
| | | KAINLILDELWHTNDNQIAIFNRLKLVPKKVDLSQQKEIPTT | |
| | | LVDDFILSPVVKRSFIQSIKVINAIIKKYGLPNDIIIELARE | |
| | | KNSKDAQKMINEMQKRNRQTNERIEEIIRTTGKENAKYLIEK | |
| | | IKLHDMQEGKCLYSLEAIPLEDLLNNPFNYEVDHIIPRSVSF | |
| | | DNSFNNKVLVKQEENSKKGNRTPFQYLSSSDSKISYETFKKH | |
| | | ILNLAKGKGRISKTKKEYLLEERDINRFSVQKDFINRNLVDT | |
| | | RYATRGLMNLLRSYFRVNNLDVKVKSINGGFTSFLRRKWKFK | |
| | | KERNKGYKHHAEDALIIANADFIFKEWKKLDKAKKVMENQME | |
| | | EEKQAESMPEIETEQEYKEIFITPHQIKHIKDFKDYKYSHRV | |
| | | DKKPNRELINDTLYSTRKDDKGNTLIVNNLNGLYDKDNDKLK | |
| | | KLINKSPEKLLMYHHDPQTYQKLKLIMEQYGDEKNPLYKYYE | |
| | | ETGNYLTKYSKKDNGPVIKKIKYYGNKLNAHLDITDDYPNSR | |
| | | NKVVKLSLKPYRFDVYLDNGVYKFVTVKNLDVIKKENYYEVN | |
| | | SKCYEEAKKLKKISNQAEFIASFYNNDLIKINGELYRVIGVN | |
| | | NDLLNRIEVNMIDITYREYLENMNDKRPPRIIKTIASKTQSI | |
| | | KKYSTDILGNLYEVKSKKHPQIIKKG | |
| pBE0022 | 23 | SEVEFSHEYWMRHALTLAKRARDEREVPVGAVLVLNNRVIGE | >90% |
| | | GWNRAIGLHDPTAHAEIMALRQGGLVMQNYRLIDATLYVTFE | (A8) |
| | | PCVMCAGAMIHSRIGRVVFGVRNSKRGAAGSLMNVLNYPGMN | |
| | | HRVEITEGILADECAALLCDFYRMPRQVFNAQKKAQSSINSG | |
| | | GSSGGSSGSETPGTSESATPESSGGSSGGSKRNYILGLAIGI | |
| | | TSVGYGIIDYETRDVIDAGVRLFKEANVENNEGRRSKRGARR | |
| | | LKRRRRHRIQRVKKLLFDYNLLTDHSELSGINPYEARVKGLS | |
| | | QKLSEEEFSAALLHLAKRRGVHNVNEVTGNELSTKEQISRNS | |
| | | KALEEKYVAELQLERLKKDGEVRGSINRFKTSDYVKEAKQLL | |
| | | KVQKAYHQLDQSFIDTYIDLLETRRTYYEGPGEGSPFGWKDI | |
| | | KEWYEMLMGHCTYFPEELRSVKYAYNADLYNALNDLNNLVIT | |
| | | RDENEKLEYYEKFQIIENVFKQKKKPTLKQIAKEILVNEEDI | |
| | | KGYRVTSTGKPEFTNLKVYHDIKDITARKEIIENAELLDQIA | |
| | | KILTIYQSSEDIQEELTNLNSELTQEEIEQISNLKGYTGTHN | |
| | | LSLKAINLILDELWHTNDNQIAIFNRLKLVPKKVDLSQQKEI | |
| | | PTTLVDDFILSPVVKRSFIQSIKVINAIIKKYGLPNDIIIEL | |
| | | AREKNSKDAQKMINEMQKRNRQTNERIEEIIRTTGKENAKYL | |
| | | IEKIKLHDMQEGKCLYSLEAIPLEDLLNNPFNYEVDHIIPRS | |
| | | VSFDNSFNNKVLVKQEENSKKGNRTPFQYLSSSDSKISYETF | |
| | | KKHILNLAKGKGRISKTKKEYLLEERDINRFSVQKDFINRNL | |
| | | VDTRYATRGLMNLLRSYFRVNNLDVKVKSINGGFTSFLRRKW | |
| | | KFKKERNKGYKHHAEDALIIANADFIFKEWKKLDKAKKVMEN | |
| | | QMFEEKQAESMPEIETEQEYKEIFITPHQIKHIKDFKDYKYS | |
| | | HRVDKKPNRELINDTLYSTRKDDKGNTLIVNNLNGLYDKDND | |
| | | KLKKLINKSPEKLLMYHHDPQTYQKLKLIMEQYGDEKNPLYK | |
| | | YYEETGNYLTKYSKKDNGPVIKKIKYYGNKLNAHLDITDDYP | |
| | | NSRNKVVKLSLKPYRFDVYLDNGVYKFVTVKNLDVIKKENYY | |
| | | EVNSKCYEEAKKLKKISNQAEFIASFYNNDLIKINGELYRVI | |
| | | GVNNDLLNRIEVNMIDITYREYLENMNDKRPPRIIKTIASKT | |
| | | QSIKKYSTDILGNLYEVKSKKHPQIIKKG | |
| pBE0024 | 24 | SEVEFSHEYWMRHALTLAKRARDEREVPVGAVLVLNNRVIGE | 75-90% |
| | | GWNRAIGLHDPTAHAEIMALRQGGLVMQNYRLIDATLYVTFE | (A8) |
| | | PCVMCAGAMIHSRIGRVVFGVRNSKRGAAGSLMNVLNYPGMN | |
| | | HRVEITEGILADECAALLCDFYRMPRQVFNAQKKAQSSINSG | |
| | | GSSGGSSGSETPGTSESATPESSGGSSGGSKRNYILGLAIGI | |
| | | TSVGYGIIDYETRDVIDAGVRLFKEANVENNEGRRSKRGARR | |
| | | LKRRRRHRIQRVKKLLFDYNLLTDHSELSGINPYEARVKGLS | |
| | | QKLSEEEFSAALLHLAKRRGVHNVNEVELSTKEQISRNSKAL | |
| | | EEKYVAELQLERLKKDGEVRGSINRFKTSDYVKEAKQLLKVQ | |
| | | KAYHQLDQSFIDTYIDLLETRRTYYEGPGEGSPFGWKDIKEW | |
| | | YEMLMGHCTYFPEELRSVKYAYNADLYNALNDLNNLVITRDE | |
| | | NEKLEYYEKFQIIENVFKQKKKPTLKQIAKEILVNEEDIKGY | |
| | | RVTSTGKPEFTNLKVYHDIKDITARKEIIENAELLDQIAKIL | |
| | | TIYQSSEDIQEELTNLNSELTQEEIEQISNLKGYTGTHNLSL | |
| | | KAINLILDELWHTNDNQIAIFNRLKLVPKKVDLSQQKEIPTT | |
| | | LVDDFILSPVVKRSFIQSIKVINAIIKKYGLPNDIIIELARE | |
| | | KNSKDAQKMINEMQKRNRQTNERIEEIIRTTGKENAKYLIEK | |
| | | IKLHDMQEGKCLYSLEAIPLEDLLNNPFNYEVDHIIPRSVSF | |
| | | DNSFNNKVLVKQEENSKKGNRTPFQYLSSSDSKISYETFKKH | |
| | | ILNLAKGKGRISKTKKEYLLEERDINRFSVQKDFINRNLVDT | |
| | | RYATRGLMNLLRSYFRVNNLDVKVKSINGGFTSFLRRKWKFK | |
| | | KERNKGYKHHAEDALIIANADFIFKEWKKLDKAKKVMENQME | |
| | | EEKQAESMPEIETEQEYKEIFITPHQIKHIKDFKDYKYSHRV | |
| | | DKKPNRELINDTLYSTRKDDKGNTLIVNNLNGLYDKDNDKLK | |
| | | KLINKSPEKLLMYHHDPQTYQKLKLIMEQYGDEKNPLYKYYE | |
| | | ETGNYLTKYSKKDNGPVIKKIKYYGNKLNAHLDITDDYPNSR | |
| | | NKVVKLSLKPYRFDVYLDNGVYKFVTVKNLDVIKKENYYEVN | |
| | | SKCYEEAKKLKKISNQAEFIASFYNNDLIKINGELYRVIGVN | |
| | | NDLLNRIEVNMIDITYREYLENMNDKRPPRIIKTIASKTQSI | |
| | | KKYSTDILGNLYEVKSKKHPQIIKKG | |
| pBE0025 | 25 | SEVEFSHEYWMRHALTLAKRARDEREVPVGAVLVLNNRVIGE | >90% |
| | | GWNRAIGLHDPTAHAEIMALRQGGLVMQNYRLIDATLYVTFE | (A8) |
| | | PCVMCAGAMIHSRIGRVVFGVRNSKRGAAGSLMNVLNYPGMN | |
| | | HRVEITEGILADECAALLCDFYRMPRQVFNAQKKAQSSINSG | |
| | | GSSGGSSGSETPGTSESATPESSGGSSGGSKRNYILGLAIGI | |
| | | TSVGYGIIDYETRDVIDAGVRLFKEANVENNEGRRSKRGARR | |
| | | LKRRRRHRIQRVKKLLFDYNLLTDHSELSGINPYEARVKGLS | |
| | | QKLSEEEFSAALLHLAKRRGVHNVNEVEEDTGNEEQISRNSK | |
| | | ALEEKYVAELQLERLKKDGEVRGSINRFKTSDYVKEAKQLLK | |
| | | VQKAYHQLDQSFIDTYIDLLETRRTYYEGPGEGSPFGWKDIK | |
| | | EWYEMLMGHCTYFPEELRSVKYAYNADLYNALNDLNNLVITR | |
| | | DENEKLEYYEKFQIIENVFKQKKKPTLKQIAKEILVNEEDIK | |
| | | GYRVTSTGKPEFTNLKVYHDIKDITARKEIIENAELLDQIAK | |
| | | ILTIYQSSEDIQEELTNLNSELTQEEIEQISNLKGYTGTHNL | |
| | | SLKAINLILDELWHTNDNQIAIFNRLKLVPKKVDLSQQKEIP | |
| | | TTLVDDFILSPVVKRSFIQSIKVINAIIKKYGLPNDIIIELA | |
| | | REKNSKDAQKMINEMQKRNRQTNERIEEIIRTTGKENAKYLI | |
| | | EKIKLHDMQEGKCLYSLEAIPLEDLLNNPFNYEVDHIIPRSV | |
| | | SFDNSFNNKVLVKQEENSKKGNRTPFQYLSSSDSKISYETFK | |
| | | KHILNLAKGKGRISKTKKEYLLEERDINRFSVQKDFINRNLV | |
| | | DTRYATRGLMNLLRSYFRVNNLDVKVKSINGGFTSFLRRKWK | |
| | | FKKERNKGYKHHAEDALIIANADFIFKEWKKLDKAKKVMENQ | |
| | | MFEEKQAESMPEIETEQEYKEIFITPHQIKHIKDFKDYKYSH | |
| | | RVDKKPNRELINDTLYSTRKDDKGNTLIVNNLNGLYDKDNDK | |
| | | LKKLINKSPEKLLMYHHDPQTYQKLKLIMEQYGDEKNPLYKY | |
| | | YEETGNYLTKYSKKDNGPVIKKIKYYGNKLNAHLDITDDYPN | |
| | | SRNKVVKLSLKPYRFDVYLDNGVYKFVTVKNLDVIKKENYYE | |
| | | VNSKCYEEAKKLKKISNQAEFIASFYNNDLIKINGELYRVIG | |
| | | VNNDLLNRIEVNMIDITYREYLENMNDKRPPRIIKTIASKTQ | |
| | | SIKKYSTDILGNLYEVKSKKHPQIIKKG | |
| pBE0026 | 26 | SEVEFSHEYWMRHALTLAKRARDEREVPVGAVLVLNNRVIGE | >90% |
| | | GWNRAIGLHDPTAHAEIMALRQGGLVMQNYRLIDATLYVTFE | (A8) |
| | | PCVMCAGAMIHSRIGRVVFGVRNSKRGAAGSLMNVLNYPGMN | |
| | | HRVEITEGILADECAALLCDFYRMPRQVFNAQKKAQSSINSG | |
| | | GSSGGSSGSETPGTSESATPESSGGSSGGSKRNYILGLAIGI | |
| | | TSVGYGIIDYETRDVIDAGVRLFKEANVENNEGRRSKRGARR | |
| | | LKRRRRHRIQRVKKLLFDYNLLTDHSELSGINPYEARVKGLS | |
| | | QKLSEEEFSAALLHLAKRRGVHNVNEVEEDTGNELSTKRNSK | |
| | | ALEEKYVAELQLERLKKDGEVRGSINRFKTSDYVKEAKQLLK | |
| | | VQKAYHQLDQSFIDTYIDLLETRRTYYEGPGEGSPFGWKDIK | |
| | | EWYEMLMGHCTYFPEELRSVKYAYNADLYNALNDLNNLVITR | |
| | | DENEKLEYYEKFQIIENVFKQKKKPTLKQIAKEILVNEEDIK | |
| | | GYRVTSTGKPEFTNLKVYHDIKDITARKEIIENAELLDQIAK | |
| | | ILTIYQSSEDIQEELTNLNSELTQEEIEQISNLKGYTGTHNL | |
| | | SLKAINLILDELWHTNDNQIAIFNRLKLVPKKVDLSQQKEIP | |
| | | TTLVDDFILSPVVKRSFIQSIKVINAIIKKYGLPNDIIIELA | |
| | | REKNSKDAQKMINEMQKRNRQTNERIEEIIRTTGKENAKYLI | |
| | | EKIKLHDMQEGKCLYSLEAIPLEDLLNNPFNYEVDHIIPRSV | |
| | | SFDNSFNNKVLVKQEENSKKGNRTPFQYLSSSDSKISYETFK | |
| | | KHILNLAKGKGRISKTKKEYLLEERDINRFSVQKDFINRNLV | |
| | | DTRYATRGLMNLLRSYFRVNNLDVKVKSINGGFTSFLRRKWK | |
| | | FKKERNKGYKHHAEDALIIANADFIFKEWKKLDKAKKVMENQ | |
| | | MFEEKQAESMPEIETEQEYKEIFITPHQIKHIKDFKDYKYSH | |
| | | RVDKKPNRELINDTLYSTRKDDKGNTLIVNNLNGLYDKDNDK | |
| | | LKKLINKSPEKLLMYHHDPQTYQKLKLIMEQYGDEKNPLYKY | |
| | | YEETGNYLTKYSKKDNGPVIKKIKYYGNKLNAHLDITDDYPN | |
| | | SRNKVVKLSLKPYRFDVYLDNGVYKFVTVKNLDVIKKENYYE | |
| | | VNSKCYEEAKKLKKISNQAEFIASFYNNDLIKINGELYRVIG | |
| | | VNNDLLNRIEVNMIDITYREYLENMNDKRPPRIIKTIASKTQ | |
| | | SIKKYSTDILGNLYEVKSKKHPQIIKKG | |
| pBE0027 | 57 | SEVEFSHEYWMRHALTLAKRARDEREVPVGAVLVLNNRVIGE | <10% |
| | | GWNRAIGLHDPTAHAEIMALRQGGLVMQNYRLIDATLYVTFE | (A8) |
| | | PCVMCAGAMIHSRIGRVVFGVRNSKRGAAGSLMNVLNYPGMN | |
| | | HRVEITEGILADECAALLCDFYRMPRQVFNAQKKAQSSINSG | |
| | | GSSGGSSGSETPGTSESATPESSGGSSGGSKRNYILGLAIGI | |
| | | TSVGYGIIDYETRDVIDAGVRLFKEANVENNEGRRSKRGARR | |
| | | LKRRRRHRIQRVKKLLFDYNLLTDHSELSGINPYEARVKGLS | |
| | | QKLSEEEFSAALLHLAKRRGVHNVNEVEEDTGNELSTKEQIS | |
| | | RNSKALEEKYVAELQLERLKKDGEGSINRFKTSDYVKEAKQL | |
| | | LKVQKAYHQLDQSFIDTYIDLLETRRTYYEGPGEGSPFGWKD | |
| | | IKEWYEMLMGHCTYFPEELRSVKYAYNADLYNALNDLNNLVI | |
| | | TRDENEKLEYYEKFQIIENVFKQKKKPTLKQIAKEILVNEED | |
| | | IKGYRVTSTGKPEFTNLKVYHDIKDITARKEIIENAELLDQI | |
| | | AKILTIYQSSEDIQEELTNLNSELTQEEIEQISNLKGYTGTH | |
| | | NLSLKAINLILDELWHTNDNQIAIFNRLKLVPKKVDLSQQKE | |
| | | IPTTLVDDFILSPVVKRSFIQSIKVINAIIKKYGLPNDIIIE | |
| | | LAREKNSKDAQKMINEMQKRNRQTNERIEEIIRTTGKENAKY | |
| | | LIEKIKLHDMQEGKCLYSLEAIPLEDLLNNPFNYEVDHIIPR | |
| | | SVSFDNSFNNKVLVKQEENSKKGNRTPFQYLSSSDSKISYET | |
| | | FKKHILNLAKGKGRISKTKKEYLLEERDINRFSVQKDFINRN | |
| | | LVDTRYATRGLMNLLRSYFRVNNLDVKVKSINGGFTSFLRRK | |
| | | WKFKKERNKGYKHHAEDALIIANADFIFKEWKKLDKAKKVME | |
| | | NQMFEEKQAESMPEIETEQEYKEIFITPHQIKHIKDEKDYKY | |
| | | SHRVDKKPNRELINDTLYSTRKDDKGNTLIVNNLNGLYDKDN | |
| | | DKLKKLINKSPEKLLMYHHDPQTYQKLKLIMEQYGDEKNPLY | |
| | | KYYEETGNYLTKYSKKDNGPVIKKIKYYGNKLNAHLDITDDY | |
| | | PNSRNKVVKLSLKPYRFDVYLDNGVYKFVTVKNLDVIKKENY | |
| | | YEVNSKCYEEAKKLKKISNQAEFIASFYNNDLIKINGELYRV | |
| | | IGVNNDLLNRIEVNMIDITYREYLENMNDKRPPRIIKTIASK | |
| | | TQSIKKYSTDILGNLYEVKSKKHPQIIKKG | |
| pBE0028 | 58 | SEVEFSHEYWMRHALTLAKRARDEREVPVGAVLVLNNRVIGE | <10% |
| | | GWNRAIGLHDPTAHAEIMALRQGGLVMQNYRLIDATLYVTFE | (A8) |
| | | PCVMCAGAMIHSRIGRVVFGVRNSKRGAAGSLMNVLNYPGMN | |
| | | HRVEITEGILADECAALLCDFYRMPRQVFNAQKKAQSSINSG | |
| | | GSSGGSSGSETPGTSESATPESSGGSSGGSKRNYILGLAIGI | |
| | | TSVGYGIIDYETRDVIDAGVRLFKEANVENNEGRRSKRGARR | |
| | | LKRRRRHRIQRVKKLLFDYNLLTDHSELSGINPYEARVKGLS | |
| | | QKLSEEEFSAALLHLAKRRGVHNVNEVEEDTGNELSTKEQIS | |
| | | RNSKALEEKYVAELQLERLKKDGEVRGSINRFKTSDYVKEAK | |
| | | VQKAYHQLDQSFIDTYIDLLETRRTYYEGPGEGSPFGWKDIK | |
| | | EWYEMLMGHCTYFPEELRSVKYAYNADLYNALNDLNNLVITR | |
| | | DENEKLEYYEKFQIIENVFKQKKKPTLKQIAKEILVNEEDIK | |
| | | GYRVTSTGKPEFTNLKVYHDIKDITARKEIIENAELLDQIAK | |
| | | ILTIYQSSEDIQEELTNLNSELTQEEIEQISNLKGYTGTHNL | |
| | | SLKAINLILDELWHTNDNQIAIFNRLKLVPKKVDLSQQKEIP | |
| | | TTLVDDFILSPVVKRSFIQSIKVINAIIKKYGLPNDIIIELA | |
| | | REKNSKDAQKMINEMQKRNRQTNERIEEIIRTTGKENAKYLI | |
| | | EKIKLHDMQEGKCLYSLEAIPLEDLLNNPFNYEVDHIIPRSV | |
| | | SFDNS FNNKVLVKQEENSKKGNRTPFQYLSSSDSKISYETFK | |
| | | KHILNLAKGKGRISKTKKEYLLEERDINRFSVQKDFINRNLV | |
| | | DTRYATRGLMNLLRSYFRVNNLDVKVKSINGGFTSFLRRKWK | |
| | | FKKERNKGYKHHAEDALIIANADFIFKEWKKLDKAKKVMENQ | |
| | | MFEEKQAESMPEIETEQEYKEIFITPHQIKHIKDFKDYKYSH | |
| | | RVDKKPNRELINDTLYSTRKDDKGNTLIVNNLNGLYDKDNDK | |
| | | LKKLINKSPEKLLMYHHDPQTYQKLKLIMEQYGDEKNPLYKY | |
| | | YEETGNYLTKYSKKDNGPVIKKIKYYGNKLNAHLDITDDYPN | |
| | | SRNKVVKLSLKPYRFDVYLDNGVYKFVTVKNLDVIKKENYYE | |
| | | VNSKCYEEAKKLKKISNQAEFIASFYNNDLIKINGELYRVIG | |
| | | VNNDLLNRIEVNMIDITYREYLENMNDKRPPRIIKTIASKTQ | |
| | | SIKKYSTDILGNLYEVKSKKHPQIIKKG | |
| Del3 | 50 | SEVEFSHEYWMRHALTLAKRARDEREVPVGAVLVLNNRVIGE | >90% |
| | | GWNRAIGLHDPTAHAEIMALRQGGLVMQNYRLIDATLYVTFE | (A8) |
| | | PCVMCAGAMIHSRIGRVVFGVRNSKRGAAGSLMNVLNYPGMN | |
| | | HRVEITEGILADECAALLCDFYRMPRQVFNAQKKAQSSINSG | |
| | | GSSGGSSGSETPGTSESATPESSGGSSGGSKRNYILGLAIGI | |
| | | TSVGYGIIDYETRDVIDAGVRLFKEANVENNEGRRSKRGARR | |
| | | LKRRRRHRIQRVKKLLFDYNLLTDHSELSGINPYEARVKGLS | |
| | | QKLSEEEFSAALLHLAKRRGVHNVNELSTKEQISRNSKALEE | |
| | | KYVAELQLERLKKDGEVRGSINRFKTSDYVKEAKQLLKVQKA | |
| | | YHQLDQSFIDTYIDLLETRRTYYEGPGEGSPFGWKDIKEWYE | |
| | | MLMGHCTYFPEELRSVKYAYNADLYNALNDLNNLVITRDENE | |
| | | KLEYYEKFQIIENVFKQKKKPTLKQIAKEILVNEEDIKGYRV | |
| | | TSTGKPEFTNLKVYHDIKDITARKEIIENAELLDQIAKILTI | |
| | | YQSSEDIQEELTNLNSELTQEEIEQISNLKGYTGTHNLSLKA | |
| | | INLILDELWHTNDNQIAIFNRLKLVPKKVDLSQQKEIPTTLV | |
| | | DDFILSPVVKRSFIQSIKVINAIIKKYGLPNDIIIELAREKN | |
| | | SKDAQKMINEMQKRNRQTNERIEEIIRTTGKENAKYLIEKIK | |
| | | LHDMQEGKCLYSLEAIPLEDLLNNPFNYEVDHIIPRSVSFDN | |
| | | SFNNKVLVKQEENSKKGNRTPFQYLSSSDSKISYETFKKHIL | |
| | | NLAKGKGRISKTKKEYLLEERDINRFSVQKDFINRNLVDTRY | |
| | | ATRGLMNLLRSYFRVNNLDVKVKSINGGFTSFLRRKWKFKKE | |
| | | RNKGYKHHAEDALIIANADFIFKEWKKLDKAKKVMENQMFEE | |
| | | KQAESMPEIETEQEYKEIFITPHQIKHIKDFKDYKYSHRVDK | |
| | | KPNRELINDTLYSTRKDDKGNTLIVNNLNGLYDKDNDKLKKL | |
| | | INKSPEKLLMYHHDPQTYQKLKLIMEQYGDEKNPLYKYYEET | |
| | | GNYLTKYSKKDNGPVIKKIKYYGNKLNAHLDITDDYPNSRNK | |
| | | VVKLSLKPYRFDVYLDNGVYKFVTVKNLDVIKKENYYEVNSK | |
| | | CYEEAKKLKKISNQAEFIASFYNNDLIKINGELYRVIGVNND | |
| | | LLNRIEVNMIDITYREYLENMNDKRPPRIIKTIASKTQSIKK | |
| | | YSTDILGNLYEVKSKKHPQIIKKG | |
| Del4 | 51 | SEVEFSHEYWMRHALTLAKRARDEREVPVGAVLVLNNRVIGE | 10-30% |
| | | GWNRAIGLHDPTAHAEIMALRQGGLVMQNYRLIDATLYVTFE | (A8) |
| | | PCVMCAGAMIHSRIGRVVFGVRNSKRGAAGSLMNVLNYPGMN | |
| | | HRVEITEGILADECAALLCDFYRMPRQVFNAQKKAQSSINSG | |
| | | GSSGGSSGSETPGTSESATPESSGGSSGGSKRNYILGLAIGI | |
| | | TSVGYGIIDYETRDVIDAGVRLFKEANVENNEGRRSKRGARR | |
| | | LKRRRRHRIQRVKKLLFDYNLLTDHSELSGINPYEARVKGLS | |
| | | QKLSEEEFSAALLHLAKRRGVHNVNEVEEDTGNELSTKEQIS | |
| | | RNSKALEEKYVAELQLERLKKDGEVRGSINRFKTSDYVKEAK | |
| | | QLLKVQKAYHQLDQSFIDTYIDLLETRRTYYEGPGEGSPFGW | |
| | | KDIKEWYEMLMGHCTYFPEELKKVDLSQQKEIPTTLVDDFIL | |
| | | SPVVKRSFIQSIKVINAIIKKYGLPNDIIIELAREKNSKDAQ | |
| | | KMINEMQKRNRQTNERIEEIIRTTGKENAKYLIEKIKLHDMQ | |
| | | EGKCLYSLEAIPLEDLLNNPFNYEVDHIIPRSVSFDNSENNK | |
| | | VLVKQEENSKKGNRTPFQYLSSSDSKISYETFKKHILNLAKG | |
| | | KGRISKTKKEYLLEERDINRFSVQKDFINRNLVDTRYATRGL | |
| | | MNLLRSYFRVNNLDVKVKSINGGFTSFLRRKWKFKKERNKGY | |
| | | KHHAEDALIIANADFIFKEWKKLDKAKKVMENQMFEEKQAES | |
| | | MPEIETEQEYKEIFITPHQIKHIKDFKDYKYSHRVDKKPNRE | |
| | | LINDTLYSTRKDDKGNTLIVNNLNGLYDKDNDKLKKLINKSP | |
| | | EKLLMYHHDPQTYQKLKLIMEQYGDEKNPLYKYYEETGNYLT | |
| | | KYSKKDNGPVIKKIKYYGNKLNAHLDITDDYPNSRNKVVKLS | |
| | | LKPYRFDVYLDNGVYKFVTVKNLDVIKKENYYEVNSKCYEEA | |
| | | KKLKKISNQAEFIASFYNNDLIKINGELYRVIGVNNDLLNRI | |
| | | EVNMIDITYREYLENMNDKRPPRIIKTIASKTQSIKKYSTDI | |
| | | LGNLYEVKSKKHPQIIKKG | |
| Del5 | 52 | SEVEFSHEYWMRHALTLAKRARDEREVPVGAVLVLNNRVIGE | 50-75% |
| | | GWNRAIGLHDPTAHAEIMALRQGGLVMQNYRLIDATLYVTFE | (A8) |
| | | PCVMCAGAMIHSRIGRVVFGVRNSKRGAAGSLMNVLNYPGMN | |
| | | HRVEITEGILADECAALLCDFYRMPRQVFNAQKKAQSSINSG | |
| | | GSSGGSSGSETPGTSESATPESSGGSSGGSKRNYILGLAIGI | |
| | | TSVGYGIIDYETRDVIDAGVRLFKEANVENNEGRRSKRGARR | |
| | | LKRRRRHRIQRVKKLLFDYNLLNPYEARVKGLSQKLSEEEFS | |
| | | AALLHLAKRRGVHNVNEVEEDTGNELSTKEQISRNSKALEEK | |
| | | YVAELQLERLKKDGEVRGSINRFKTSDYVKEAKQLLKVQKAY | |
| | | HQLDQSFIDTYIDLLETRRTYYEGPGEGSPFGWKDIKEWYEM | |
| | | LMGHCTYFPEELRSVKYAYNADLYNALNDLNNLVITRDENEK | |
| | | LEYYEKFQIIENVFKQKKKPTLKQIAKEILVNEEDIKGYRVT | |
| | | STGKPEFTNLKVYHDIKDITARKEIIENAELLDQIAKILTIY | |
| | | QSSEDIQEELTNLNSELTQEEIEQISNLKGYTGTHNLSLKAI | |
| | | NLILDELWHTNDNQIAIFNRLKLVPKKVDLSQQKEIPTTLVD | |
| | | DFILSPVVKRSFIQSIKVINAIIKKYGLPNDIIIELAREKNS | |
| | | KDAQKMINEMQKRNRQTNERIEEIIRTTGKENAKYLIEKIKL | |
| | | HDMQEGKCLYSLEAIPLEDLLNNPFNYEVDHIIPRSVSFDNS | |
| | | FNNKVLVKQEENSKKGNRTPFQYLSSSDSKISYETFKKHILN | |
| | | LAKGKGRISKTKKEYLLEERDINRFSVQKDFINRNLVDTRYA | |
| | | TRGLMNLLRSYFRVNNLDVKVKSINGGFTSFLRRKWKFKKER | |
| | | NKGYKHHAEDALIIANADFIFKEWKKLDKAKKVMENQMFEEK | |
| | | QAESMPEIETEQEYKEIFITPHQIKHIKDFKDYKYSHRVDKK | |
| | | PNRELINDTLYSTRKDDKGNTLIVNNLNGLYDKDNDKLKKLI | |
| | | NKSPEKLLMYHHDPQTYQKLKLIMEQYGDEKNPLYKYYEETG | |
| | | NYLTKYSKKDNGPVIKKIKYYGNKLNAHLDITDDYPNSRNKV | |
| | | VKLSLKPYRFDVYLDNGVYKFVTVKNLDVIKKENYYEVNSKC | |
| | | YEEAKKLKKISNQAEFIASFYNNDLIKINGELYRVIGVNNDL | |
| | | LNRIEVNMIDITYREYLENMNDKRPPRIIKTIASKTQSIKKY | |
| | | STDILGNLYEVKSKKHPQIIKKG | |
| pBE0029 | 27 | SEVEFSHEYWMRHALTLAKRARDEREVPVGAVLVLNNRVIGE | >90% |
| | | GWNRAIGLHDPTAHAEIMALRQGGLVMQNYRLIDATLYVTFE | (A8) |
| | | PCVMCAGAMIHSRIGRVVFGVRNSKRGAAGSLMNVLNYPGMN | |
| | | HRVEITEGILADECAALLCDFYRMPRQVFNAQKKAQSSINSG | |
| | | GSSGGSSGSETPGTSESATPESSGGSSGGSKRNYILGLAIGI | |
| | | TSVGYGIIDYETRDVIDAGVRLFKEANVENNEGRRSKRGARR | |
| | | LKRRRRHRIQRVKKLLFDYNLLTDHSELSGINPYEARVKGLS | |
| | | QKLSEEEFSAALLHLAKRRGVHNVNEVEEDTGNELSTKEQIS | |
| | | RNSKALEEKYVAELQLERLKKDGEVRGSINRFKTSDYVKEAK | |
| | | QLLKVQKAYHQLDQSFIDTYIDLLETRRTYYEGPGEGSPFGW | |
| | | KDIKEWYEMLMGHCTYFPEELRSVKYAYNADLYNALNDLNNL | |
| | | VITRDENEKLEYYEKFQIIENVFKQKKKPTLKQIAKEILVNE | |
| | | EDIKGYRVTSTGKPEFTNLKVYHDIKDITARKEIIENAELLD | |
| | | QIAKILTIYQSSEDIQEELTNLNSELTQEEIEQISNLKGYTG | |
| | | THNLSLKAINLILDELWHTNDNQIAIFNRLKLVPKKVDLSQQ | |
| | | KEIPTTLVDDFILSPVVKRSFIQSIKVINAIIKKYGLPNDII | |
| | | IELAREKNSKDAQKMINEMQKRNRQTNERIEEIIRTTGKENA | |
| | | KYLIEKIKLHDMQEGKCLYSLEAIPLEDLLNNPFNYEVDHII | |
| | | PRSVSFDNSFNNKVLVKQEENSKKGNRTPFQYLSSSDSKISY | |
| | | ETFKKHILNLAKGKGRISKTKKEYLLEERDINRFSVQKDFIN | |
| | | RNLVDTRYATRGLMNLLRSYFRVNNLDVKVKSINGGFTSFLR | |
| | | RKWKFKKERNKGYKHHAEDALIIANADFIFKEWMFEEKQAES | |
| | | MPEIETEQEYKEIFITPHQIKHIKDFKDYKYSHRVDKKPNRE | |
| | | LINDTLYSTRKDDKGNTLIVNNLNGLYDKDNDKLKKLINKSP | |
| | | EKLLMYHHDPQTYQKLKLIMEQYGDEKNPLYKYYEETGNYLT | |
| | | KYSKKDNGPVIKKIKYYGNKLNAHLDITDDYPNSRNKVVKLS | |
| | | LKPYRFDVYLDNGVYKFVTVKNLDVIKKENYYEVNSKCYEEA | |
| | | KKLKKISNQAEFIASFYNNDLIKINGELYRVIGVNNDLLNRI | |
| | | EVNMIDITYREYLENMNDKRPPRIIKTIASKTQSIKKYSTDI | |
| | | LGNLYEVKSKKHPQIIKKG | |
| Del2 | 59 | SEVEFSHEYWMRHALTLAKRARDEREVPVGAVLVLNNRVIGE | <10% |
| | | GWNRAIGLHDPTAHAEIMALRQGGLVMQNYRLIDATLYVTFE | (A8) |
| | | PCVMCAGAMIHSRIGRVVFGVRNSKRGAAGSLMNVLNYPGMN | |
| | | HRVEITEGILADECAALLCDFYRMPRQVFNAQKKAQSSINSG | |
| | | GSSGGSSGSETPGTSESATPESSGGSSGGSKRNYILGLAIGI | |
| | | TSVGYGIIDYETRDVIDAGVRLFKEANVENNEGRRSKRGARR | |
| | | LKRRRRHRIQRVKKLLFDYNLLTDHSELSGINPYEARVKGLS | |
| | | QKLSEEEFSAALLHLAKRRGVHNVNEVEEDTGNELSTKEQIS | |
| | | RNSKALEEKYVAELQLERLKKDGEVRGSINRFKTSDYVKEAK | |
| | | QLLKVQKAYHQLDQSFIDTYIDLLETRRTYYEGPGEGSPFGW | |
| | | KDIKEWYEMLMGHCTYFPEELRSVKYAYNADLYNALNDLNNL | |
| | | VITRDENEKLEYYEKFQIIENVFKQKKKPTLKQIAKEILVNE | |
| | | EDIKGYRVTSTGKPEFTNLKVYHDIKDITARKEIIENAELLD | |
| | | QIAKILTIYQSSEDIQEELTNLNSELTQEEIEQISNLKGYTG | |
| | | THNLSLKAINLILDELWHTNDNQIAIFNRLKLVPKKVDLSQQ | |
| | | KEIPTTLVDDFILSPVVKRSFIQSIKVINAIIKKYGLPNDII | |
| | | IELAREKNSKDAQKMINEMQKRNRQTNERIEEIIRTTGKENA | |
| | | KYLIEKIKLHDMQEGKCLYSLEAIPLEDLLNNPFNYEVDHII | |
| | | PRSVSFDNSFNNKVLVKQEENSKKGNRTPFQYLSSSDSKISY | |
| | | ETFKKHILNLAKGKGRISKTKKEYLLEERDINRFSVQKDFIN | |
| | | RNLVDTRYATRGLMNLLRSYFRVNNLDVKVKSINGGFTSFLR | |
| | | RKWKFKKERNKGYKHHAEDALIIANADFIYSHRVDKKPNREL | |
| | | INDTLYSTRKDDKGNTLIVNNLNGLYDKDNDKLKKLINKSPE | |
| | | KLLMYHHDPQTYQKLKLIMEQYGDEKNPLYKYYEETGNYLTK | |
| | | YSKKDNGPVIKKIKYYGNKLNAHLDITDDYPNSRNKVVKLSL | |
| | | KPYRFDVYLDNGVYKFVTVKNLDVIKKENYYEVNSKCYEEAK | |
| | | KLKKISNQAEFIASFYNNDLIKINGELYRVIGVNNDLLNRIE | |
| | | VNMIDITYREYLENMNDKRPPRIIKTIASKTQSIKKYSTDIL | |
| | | GNLYEVKSKKHPQIIKKG | |
| Del6 | 53 | SEVEFSHEYWMRHALTLAKRARDEREVPVGAVLVLNNRVIGE | >90% |
| | | GWNRAIGLHDPTAHAEIMALRQGGLVMQNYRLIDATLYVTFE | (A8) |
| | | PCVMCAGAMIHSRIGRVVFGVRNSKRGAAGSLMNVLNYPGMN | |
| | | HRVEITEGILADECAALLCDFYRMPRQVFNAQKKAQSSINSG | |
| | | GSSGGSSGSETPGTSESATPESSGGSSGGSKRNYILGLAIGI | |
| | | TSVGYGIIDYETRDVIDAGVRLFKEANVENNEGRRSKRGARR | |
| | | LKRRRRHRIQRVKKLLFDYNLLTDHSELSGINPYEARVKGLS | |
| | | QKLSEEEFSAALLHLAKRRGVHNVNEVEEDTGNELSTKEQIS | |
| | | RNSKALEEKYVAELQLERLKKDGEVRGSINRFKTSDYVKEAK | |
| | | QLLKVQKAYHQLDQSFIDTYIDLLETRRTYYEGPGEGSPFGW | |
| | | KDIKEWYEMLMGHCTYFPEELRSVKYAYNADLYNALNDLNNL | |
| | | VITRDENEKLEYYEKFQIIENVFKQKKKPTLKQIAKEILVNE | |
| | | EDIKGYRVTSTGKPEFTNLKVYHDIKDITARKEIIENAELLD | |
| | | QIAKILTIYQSSEDIQEELTNLNSELTQEEIEQISNLKGYTG | |
| | | THNLSLKAINLILDELWHTNDNQIAIFNRLKLVPKKVDLSQQ | |
| | | KEIPTTLVDDFILSPVVKRSFIQSIKVINAIIKKYGLPNDII | |
| | | IELAREKNSKDAQKMINEMQKRNRQTNERIEEIIRTTGKENA | |
| | | KYLIEKIKLHDMQEGKCLYSLEAIPLEDLLNNPFNYEVDHII | |
| | | PRSVSFDNSFNNKVLVKQEENSKKGNRTPFQYLSSSDSKISY | |
| | | ETFKKHILNLAKGKGRISKTKKEYLLEERDINRFSVQKDFIN | |
| | | RNLVDTRYATRGLMNLLRSYFRVNNLDVKVKSINGGFTSFLR | |
| | | RKWFKKERNKGYKHHAEDALIIANADFIFKEWLDKAKKVMEN | |
| | | QMFEEKQAESMPEIETEQEYKEIFITPHQIKHIKDFKDYKYS | |
| | | HRVDKKPNRELINDTLYSTRKDDKGNTLIVNNLNGLYDKDND | |
| | | KLKKLINKSPEKLLMYHHDPQTYQKLKLIMEQYGDEKNPLYK | |
| | | YYEETGNYLTKYSKKDNGPVIKKIKYYGNKLNAHLDITDDYP | |
| | | NSRNKVVKLSLKPYRFDVYLDNGVYKFVTVKNLDVIKKENYY | |
| | | EVNSKCYEEAKKLKKISNQAEFIASFYNNDLIKINGELYRVI | |
| | | GVNNDLLNRIEVNMIDITYREYLENMNDKRPPRIIKTIASKT | |
| | | QSIKKYSTDILGNLYEVKSKKHPQIIKKG | |
| Del7 | 54 | SEVEFSHEYWMRHALTLAKRARDEREVPVGAVLVLNNRVIGE | >90% |
| | | GWNRAIGLHDPTAHAEIMALRQGGLVMQNYRLIDATLYVTFE | (A8) |
| | | PCVMCAGAMIHSRIGRVVFGVRNSKRGAAGSLMNVLNYPGMN | |
| | | HRVEITEGILADECAALLCDFYRMPRQVFNAQKKAQSSINSG | |
| | | GSSGGSSGSETPGTSESATPESSGGSSGGSKRNYILGLAIGI | |
| | | TSVGYGIIDYETRDVIDAGVRLFKEANVENNEGRRSKRGARR | |
| | | LKRRRRHRIQRVKKLLFDYNLLTDHSELSGINPYEARVKGLS | |
| | | QKLSEEEFSAALLHLAKRRGVHNVNEVEEDTGNELSTKEQIS | |
| | | RNSKALEEKYVAELQLERLKKDGEVRGSINRFKTSDYVKEAK | |
| | | QLLKVQKAYHQLDQSFIDTYIDLLETRRTYYEGPGEGSPFGW | |
| | | KDIKEWYEMLMGHCTYFPEELRSVKYAYNADLYNALNDLNNL | |
| | | VITRDENEKLEYYEKFQIIENVFKQKKKPTLKQIAKEILVNE | |
| | | EDIKGYRVTSTGKPEFTNLKVYHDIKDITARKEIIENAELLD | |
| | | QIAKILTIYQSSEDIQEELTNLNSELTQEEIEQISNLKGYTG | |
| | | THNLSLKAINLILDELWHTNDNQIAIFNRLKLVPKKVDLSQQ | |
| | | KEIPTTLVDDFILSPVVKRSFIQSIKVINAIIKKYGLPNDII | |
| | | IELAREKNSKDAQKMINEMQKRNRQTNERIEEIIRTTGKENA | |
| | | KYLIEKIKLHDMQEGKCLYSLEAIPLEDLLNNPFNYEVDHII | |
| | | PRSVSFDNSFNNKVLVKQEENSKKGNRTPFQYLSSSDSKISY | |
| | | ETFKKHILNLAKGKGRISKTKKEYLLEERDINRFSVQKDFIN | |
| | | RNLVDTRYATRGLMNLLRSYFRVNNLDVKVKSINGGFTSFLR | |
| | | RKWKFKKERNKGYKHHAEDALIIANADFIFKEWLDKAKKVME | |
| | | NQMFEEKQAESMPEIETEQEYKEIFITPHQIKHIKDFKDYKY | |
| | | SHRVDKKPNRELINDTLYSTRKDDKGNTLIVNNLNGLYDKDN | |
| | | DKLKKLINKSPEKLLMYHHDPQTYQKLKLIMEQYGDEKNPLY | |
| | | KYYEETGNYLTKYSKKDNGPVIKKIKYYGNKLNAHLDITDDY | |
| | | PNSRNKVVKLSLKPYRFDVYLDNGVYKFVTVKNLDVIKKENY | |
| | | YEVNSKCYEEAKKLKKISNQAEFIASFYNNDLIKINGELYRV | |
| | | IGVNNDLLNRIEVNMIDITYREYLENMNDKRPPRIIKTIASK | |
| | | TQSIKKYSTDILGNLYEVKSKKHPQIIKKG | |
| Del8 | 55 | SEVEFSHEYWMRHALTLAKRARDEREVPVGAVLVLNNRVIGE | >90% |
| | | GWNRAIGLHDPTAHAEIMALRQGGLVMQNYRLIDATLYVTFE | (A8) |
| | | PCVMCAGAMIHSRIGRVVFGVRNSKRGAAGSLMNVLNYPGMN | |
| | | HRVEITEGILADECAALLCDFYRMPRQVFNAQKKAQSSINSG | |
| | | GSSGGSSGSETPGTSESATPESSGGSSGGSKRNYILGLAIGI | |
| | | TSVGYGIIDYETRDVIDAGVRLFKEANVENNEGRRSKRGARR | |
| | | LKRRRRHRIQRVKKLLFDYNLLTDHSELSGINPYEARVKGLS | |
| | | QKLSEEEFSAALLHLAKRRGVHNVNEVEEDTGNELSTKEQIS | |
| | | RNSKALEEKYVAELQLERLKKDGEVRGSINRFKTSDYVKEAK | |
| | | QLLKVQKAYHQLDQSFIDTYIDLLETRRTYYEGPGEGSPFGW | |
| | | KDIKEWYEMLMGHCTYFPEELRSVKYAYNADLYNALNDLNNL | |
| | | VITRDENEKLEYYEKFQIIENVFKQKKKPTLKQIAKEILVNE | |
| | | EDIKGYRVTSTGKPEFTNLKVYHDIKDITARKEIIENAELLD | |
| | | QIAKILTIYQSSEDIQEELTNLNSELTQEEIEQISNLKGYTG | |
| | | THNLSLKAINLILDELWHTNDNQIAIFNRLKLVPKKVDLSQQ | |
| | | KEIPTTLVDDFILSPVVKRSFIQSIKVINAIIKKYGLPNDII | |
| | | IELAREKNSKDAQKMINEMQKRNRQTNERIEEIIRTTGKENA | |
| | | KYLIEKIKLHDMQEGKCLYSLEAIPLEDLLNNPFNYEVDHII | |
| | | PRSVSFDNSFNNKVLVKQEENSKKGNRTPFQYLSSSDSKISY | |
| | | ETFKKHILNLAKGKGRISKTKKEYLLEERDINRFSVQKDFIN | |
| | | RNLVDTRYATRGLMNLLRSYFRVNNLDVKVKSINGGFTSFLR | |
| | | RKFKKERNKGYKHHAEDALIIANADFIFKEWLDKAKKVMENQ | |
| | | MFEEKQAESMPEIETEQEYKEIFITPHQIKHIKDFKDYKYSH | |
| | | RVDKKPNRELINDTLYSTRKDDKGNTLIVNNLNGLYDKDNDK | |
| | | LKKLINKSPEKLLMYHHDPQTYQKLKLIMEQYGDEKNPLYKY | |
| | | YEETGNYLTKYSKKDNGPVIKKIKYYGNKLNAHLDITDDYPN | |
| | | SRNKVVKLSLKPYRFDVYLDNGVYKFVTVKNLDVIKKENYYE | |
| | | VNSKCYEEAKKLKKISNQAEFIASFYNNDLIKINGELYRVIG | |
| | | VNNDLLNRIEVNMIDITYREYLENMNDKRPPRIIKTIASKTQ | |
| | | SIKKYSTDILGNLYEVKSKKHPQIIKKG | |
| Del9 | 56 | SEVEFSHEYWMRHALTLAKRARDEREVPVGAVLVLNNRVIGE | >90% |
| | | GWNRAIGLHDPTAHAEIMALRQGGLVMQNYRLIDATLYVTFE | (A8) |
| | | PCVMCAGAMIHSRIGRVVFGVRNSKRGAAGSLMNVLNYPGMN | |
| | | HRVEITEGILADECAALLCDFYRMPRQVFNAQKKAQSSINSG | |
| | | GSSGGSSGSETPGTSESATPESSGGSSGGSKRNYILGLAIGI | |
| | | TSVGYGIIDYETRDVIDAGVRLFKEANVENNEGRRSKRGARR | |
| | | LKRRRRHRIQRVKKLLFDYNLLTDHSELSGINPYEARVKGLS | |
| | | QKLSEEEFSAALLHLAKRRGVHNVNEVEEDTGNELSTKEQIS | |
| | | RNSKALEEKYVAELQLERLKKDGEVRGSINRFKTSDYVKEAK | |
| | | QLLKVQKAYHQLDQSFIDTYIDLLETRRTYYEGPGEGSPFGW | |
| | | KDIKEWYEMLMGHCTYFPEELRSVKYAYNADLYNALNDLNNL | |
| | | VITRDENEKLEYYEKFQIIENVFKQKKKPTLKQIAKEILVNE | |
| | | EDIKGYRVTSTGKPEFTNLKVYHDIKDITARKEIIENAELLD | |
| | | QIAKILTIYQSSEDIQEELTNLNSELTQEEIEQISNLKGYTG | |
| | | THNLSLKAINLILDELWHTNDNQIAIFNRLKLVPKKVDLSQQ | |
| | | KEIPTTLVDDFILSPVVKRSFIQSIKVINAIIKKYGLPNDII | |
| | | IELAREKNSKDAQKMINEMQKRNRQTNERIEEIIRTTGKENA | |
| | | KYLIEKIKLHDMQEGKCLYSLEAIPLEDLLNNPFNYEVDHII | |
| | | PRSVSFDNSFNNKVLVKQEENSKKGNRTPFQYLSSSDSKISY | |
| | | ETFKKHILNLAKGKGRISKTKKEYLLEERDINRFSVQKDFIN | |
| | | RNLVDTRYATRGLMNLLRSYFRVNNLDVKVKSINGGFTSFLR | |
| | | RKFKKERNKGYKHHAEDALIIANADFIFKDKAKKVMENQMFE | |
| | | EKQAESMPEIETEQEYKEIFITPHQIKHIKDFKDYKYSHRVD | |
| | | KKPNRELINDTLYSTRKDDKGNTLIVNNLNGLYDKDNDKLKK | |
| | | LINKSPEKLLMYHHDPQTYQKLKLIMEQYGDEKNPLYKYYEE | |
| | | TGNYLTKYSKKDNGPVIKKIKYYGNKLNAHLDITDDYPNSRN | |
| | | KVVKLSLKPYRFDVYLDNGVYKFVTVKNLDVIKKENYYEVNS | |
| | | KCYEEAKKLKKISNQAEFIASFYNNDLIKINGELYRVIGVNN | |
| | | DLLNRIEVNMIDITYREYLENMNDKRPPRIIKTIASKTQSIK | |
| | | KYSTDILGNLYEVKSKKHPQIIKKG | |
| pBE0031 | 60 | SEVEFSHEYWMRHALTLAKRARDEREVPVGAVLVLNNRVIGE | <10% |
| | | GWNRAIGLHDPTAHAEIMALRQGGLVMQNYRLIDATLYVTFE | (A8) |
| | | PCVMCAGAMIHSRIGRVVFGVRNSKRGAAGSLMNVLNYPGMN | |
| | | HRVEITEGILADECAALLCDFYRMPRQVFNAQKKAQSSINSG | |
| | | GSSGGSSGSETPGTSESATPESSGGSSGGSKRNYILGLAIGI | |
| | | TSVGYGIIDYETRDVIDAGVRLFKEANVENNEGRRSKRGARR | |
| | | LKRRRRHRIQRVKKLLFDYNLLTDHSELSGINPYEARVKGLS | |
| | | QKLSEEEFSAALLHLAKRRGVHNVNEVEEDTGNELSTKEQIS | |
| | | RNSKALEEKYVAELQLERLKKDGEVRGSINRFKTSDYVKEAK | |
| | | QLLKVQKAYHQLDQSFIDTYIDLLETRRTYYEGPGEGSPFGW | |
| | | KDIKEWYEMLMGHCTYFPEELRSVKYAYNADLYNALNDLNNL | |
| | | VITRDENEKLEYYEKFQIIENVFKQKKKPTLKQIAKEILVNE | |
| | | EDIKGYRVTSTGKPEFTNLKVYHDIKDITARKEIIENAELLD | |
| | | QIAKILTIYQSSEDIQEELTNLNSELTQEEIEQISNLKGYTG | |
| | | THNLSLKAINLILDELWHTNDNQIAIFNRLKLVPKKVDLSQQ | |
| | | KEIPTTLVDDFILSPVVKRSFIQSIKVINAIIKKYGLPNDII | |
| | | IELAREKNSKDAQKMINEMQKRNRQTNERIEEIIRTTGKENA | |
| | | KYLIEKIKLHDMQEGKCLYSLEAIPLEDLLNNPFNYEVDHII | |
| | | PRSVSFDNSFNNKVLVKQEENSKKGNRTPFQYLSSSDSKISY | |
| | | ETFKKHILNLAKGKGRISKTKKEYLLEERDINRFSVQKDFIN | |
| | | RNLVDTRYATRGLMNLLRSYFRVNNLDVKVKSINGGFTSFLR | |
| | | RKWKFKKERNKGYKHHAEDALIIANADFIFKEWKKLDKAKKV | |
| | | MENQMFEEKQAESMPEIETEQEYKEIFITPHQIKHIKDFKDY | |
| | | KYSHRVDKKPNRELINDTLYSTRKDDKGNTLIVNNLNGLYDK | |
| | | DNDKLKKLINKSPEKLLMYHHDPQTYQKLKLIMEQYGDEKNP | |
| | | LYKYYEETGNYLTKYSKKDNGPVIKKIKYYGNKLNAHLDITD | |
| | | DYPNSRNKVVKLSLKPYRFDVYLDNGVYKFVTVKNLDVIKKE | |
| | | NYYEVNSKCYEEAKKLKKISNQAEFIASFYNNDLIKINGELV | |
| | | IGVNNDLLNRIEVNMIDITYREYLENMNDKRPPRIIKTIASK | |
| | | TQSIKKYSTDILGNLYEVKSKKHPQIIKKG | |
| pBE0038 | 28 | SEVEFSHEYWMRHALTLAKRARDEREVPVGAVLVLNNRVIGE | >90% |
| | | GWNRAIGLHDPTAHAEIMALRQGGLVMQNYRLIDATLYVTFE | (A8) |
| | | PCVMCAGAMIHSRIGRVVFGVRNSKRGAAGSLMNVLNYPGMN | |
| | | HRVEITEGILADECAALLCDFYRMPRQVFNAQKKAQSSINSG | |
| | | GSSGGSSGSETPGTSESATPESSGGSSGGSKRNYILGLAIGI | |
| | | TSVGYGIIDYETRDVIDAGVRLFKEANVENNEGRRSKRGARR | |
| | | LKRRRRHRIQRVKKLLFDYNLLTDHSELSGINPYEARVKGLS | |
| | | QKLSEEEFSAALLHLAKRRGVHNVNEVEKYVAELQLERLKKD | |
| | | GEVRGSINRFKTSDYVKEAKQLLKVQKAYHQLDQSFIDTYID | |
| | | LLETRRTYYEGPGEGSPFGWKDIKEWYEMLMGHCTYFPEELR | |
| | | SVKYAYNADLYNALNDLNNLVITRDENEKLEYYEKFQIIENV | |
| | | FKQKKKPTLKQIAKEILVNEEDIKGYRVTSTGKPEFTNLKVY | |
| | | HDIKDITARKEIIENAELLDQIAKILTIYQSSEDIQEELTNL | |
| | | NSELTQEEIEQISNLKGYTGTHNLSLKAINLILDELWHTNDN | |
| | | QIAIFNRLKLVPKKVDLSQQKEIPTTLVDDFILSPVVKRSFI | |
| | | QSIKVINAIIKKYGLPNDIIIELAREKNSKDAQKMINEMQKR | |
| | | NRQTNERIEEIIRTTGKENAKYLIEKIKLHDMQEGKCLYSLE | |
| | | AIPLEDLLNNPFNYEVDHIIPRSVSFDNSENNKVLVKQEENS | |
| | | KKGNRTPFQYLSSSDSKISYETFKKHILNLAKGKGRISKTKK | |
| | | EYLLEERDINRFSVQKDFINRNLVDTRYATRGLMNLLRSYER | |
| | | VNNLDVKVKSINGGFTSFLRRKWKFKKERNKGYKHHAEDALI | |
| | | IANADFIFKEWKKLDKAKKVMENQMFEEKQAESMPEIETEQE | |
| | | YKEIFITPHQIKHIKDFKDYKYSHRVDKKPNRELINDTLYST | |
| | | RKDDKGNTLIVNNLNGLYDKDNDKLKKLINKSPEKLLMYHHD | |
| | | PQTYQKLKLIMEQYGDEKNPLYKYYEETGNYLTKYSKKDNGP | |
| | | VIKKIKYYGNKLNAHLDITDDYPNSRNKVVKLSLKPYREDVY | |
| | | LDNGVYKFVTVKNLDVIKKENYYEVNSKCYEEAKKLKKISNQ | |
| | | AEFIASFYNNDLIKINGELYRVIGVNNDLLNRIEVNMIDITY | |
| | | REYLENMNDKRPPRIIKTIASKTQSIKKYSTDILGNLYEVKS | |
| | | KKHPQIIKKG | |
| pBE0039 | 61 | SEVEFSHEYWMRHALTLAKRARDEREVPVGAVLVLNNRVIGE | <10% |
| | | GWNRAIGLHDPTAHAEIMALRQGGLVMQNYRLIDATLYVTFE | (A8) |
| | | PCVMCAGAMIHSRIGRVVFGVRNSKRGAAGSLMNVLNYPGMN | |
| | | HRVEITEGILADECAALLCDFYRMPRQVFNAQKKAQSSINSG | |
| | | GSSGGSSGSETPGTSESATPESSGGSSGGSKRNYILGLAIGI | |
| | | TSVGYGIIDYETRDVIDAGVRLFKEANVENNEGRRSKRGARR | |
| | | LKRRRRHRIQRVKKLLFDYNLLTDHSLSQKLSEEEFSAALLH | |
| | | LAKRRGVHNVNEVEEDTGNELSTKEQISRNSKALEEKYVAEL | |
| | | QLERLKKDGEVRGSINRFKTSDYVKEAKQLLKVQKAYHQLDQ | |
| | | SFIDTYIDLLETRRTYYEGPGEGSPFGWKDIKEWYEMLMGHC | |
| | | TYFPEELRSVKYAYNADLYNALNDLNNLVITRDENEKLEYYE | |
| | | KFQIIENVFKQKKKPTLKQIAKEILVNEEDIKGYRVTSTGKP | |
| | | EFTNLKVYHDIKDITARKEIIENAELLDQIAKILTIYQSSED | |
| | | IQEELTNLNSELTQEEIEQISNLKGYTGTHNLSLKAINLILD | |
| | | ELWHTNDNQIAIFNRLKLVPKKVDLSQQKEIPTTLVDDFILS | |
| | | PVVKRSFIQSIKVINAIIKKYGLPNDIIIELAREKNSKDAQK | |
| | | MINEMQKRNRQTNERIEEIIRTTGKENAKYLIEKIKLHDMQE | |
| | | GKCLYSLEAIPLEDLLNNPFNYEVDHIIPRSVSEDNSENNKV | |
| | | LVKQEENSKKGNRTPFQYLSSSDSKISYETFKKHILNLAKGK | |
| | | GRISKTKKEYLLEERDINRFSVQKDFINRNLVDTRYATRGLM | |
| | | NLLRSYFRVNNLDVKVKSINGGFTSFLRRKWKFKKERNKGYK | |
| | | HHAEDALIIANADFIFKEWKKLDKAKKVMENQMFEEKQAESM | |
| | | PEIETEQEYKEIFITPHQIKHIKDFKDYKYSHRVDKKPNREL | |
| | | INDTLYSTRKDDKGNTLIVNNLNGLYDKDNDKLKKLINKSPE | |
| | | KLLMYHHDPQTYQKLKLIMEQYGDEKNPLYKYYEETGNYLTK | |
| | | YSKKDNGPVIKKIKYYGNKLNAHLDITDDYPNSRNKVVKLSL | |
| | | KPYRFDVYLDNGVYKFVTVKNLDVIKKENYYEVNSKCYEEAK | |
| | | KLKKISNQAEFIASFYNNDLIKINGELYRVIGVNNDLLNRIE | |
| | | VNMIDITYREYLENMNDKRPPRIIKTIASKTQSIKKYSTDIL | |
| | | GNLYEVKSKKHPQIIKKG | |
| pBE0040 | 29 | SEVEFSHEYWMRHALTLAKRARDEREVPVGAVLVLNNRVIGE | 75-90% |
| | | GWNRAIGLHDPTAHAEIMALRQGGLVMQNYRLIDATLYVTFE | (A8) |
| | | PCVMCAGAMIHSRIGRVVFGVRNSKRGAAGSLMNVLNYPGMN | |
| | | HRVEITEGILADECAALLCDFYRMPRQVFNAQKKAQSSINSG | |
| | | GSSGGSSGSETPGTSESATPESSGGSSGGSKRNYILGLAIGI | |
| | | TSVGYGIIDYETRDVIDAGVRLFKEANVENNEGRRSKRGARR | |
| | | LKRRRRHRIQRVKKLLFDYNLLTDHSELSGINPYEARVKGLS | |
| | | QKLSEEEFSAALLHLAKRRGVHNVNEVEEDTGNELSTKEQIS | |
| | | RNSKALEEKYVAELQLERLKKDGEVRGSINRFKTSDYVKEAK | |
| | | QLLKVQKAYHQLDQSFIDTYIDLLETRRTYYEGPGEGSPFGW | |
| | | KDIKEWYEMLMGHCTYFPEELRSVKYAYNADLYNALNDLNNL | |
| | | VITRDENEKLEYYEKFQIIENVFKQKKKPTLKQIAKEILVNE | |
| | | EDIKGYRVTSTGKPEFTNLKVYHDIKDITARKEIIENAELLD | |
| | | QIAKILTIYQSSEDIQEELTNLNSELTQEEIEQISNLKGYTG | |
| | | THNLSLKAINLILDELWHTNDNQIAIFNRLKLVPKKVDLSQQ | |
| | | KEIPTTLVDDFILSPVVKRSFIQSIKVINAIIKKYGLPNDII | |
| | | IELAREKNSKDAQKMINEMQKRNRQTNERIEEIIRTTGKENA | |
| | | KYLIEKIKLHDMQEGKCLYSLEAIPLEDLLNNPFNYEVDHII | |
| | | PRSVSFDNSFNNKVLVKQEENSKKGNRTPFQYLSSSDSKISY | |
| | | ETFKKHILNLAKGKGRISKTKKEYLLEERDINRFSVQKDFIN | |
| | | RNLVDTRYATRGLMNLLRSYFRVNNLDVKVKSINGGFTSFLR | |
| | | RKWKFKKERNKGYKHHAEDALIIANADFIFKEWKKLDKAKKV | |
| | | MENQMFEEKQAESMPEIETEQEYKEIFITPHQIKHIKDEKDY | |
| | | KYSHRVDKKPNRELINDTLYSTRKDDKGNTLIVNNLNGLYDK | |
| | | DNDKLKKLINKSPEKLLMYHHDPQTYQKLKLIMEQYGDEKNP | |
| | | LYKYYEETGNYLPVIKKIKYYGNKLNAHLDITDDYPNSRNKV | |
| | | VKLSLKPYRFDVYLDNGVYKFVTVKNLDVIKKENYYEVNSKC | |
| | | YEEAKKLKKISNQAEFIASFYNNDLIKINGELYRVIGVNNDL | |
| | | LNRIEVNMIDITYREYLENMNDKRPPRIIKTIASKTQSIKKY | |
| | | STDILGNLYEVKSKKHPQIIKKG | |
| pBE0041 | 62 | SEVEFSHEYWMRHALTLAKRARDEREVPVGAVLVLNNRVIGE | <10% |
| | | GWNRAIGLHDPTAHAEIMALRQGGLVMQNYRLIDATLYVTFE | (A8) |
| | | PCVMCAGAMIHSRIGRVVFGVRNSKRGAAGSLMNVLNYPGMN | |
| | | HRVEITEGILADECAALLCDFYRMPRQVFNAQKKAQSSINSG | |
| | | GSSGGSSGSETPGTSESATPESSGGSSGGSKRNYILGLAIGI | |
| | | TSVGYGIIDYETRDVIDAGVRLFKEANVENNEGRRSKRGARR | |
| | | LKRRRRHRIQRVKKLLFDYNLLTDHSELSGINPYEARVKGLS | |
| | | QKLSEEEFSAALLHLAKRRGVHNVNEVEEDTGNELSTKEQIS | |
| | | RNSKALEEKYVAELQLERLKKDGEVRGSINRFKTSDYVKEAK | |
| | | QLLKVQKAYHQLDQSFIDTYIDLLETRRTYYEGPGEGSPFGW | |
| | | KDIKEWYEMLMGHCTYFPEELRSVKYAYNADLYNALNDLNNL | |
| | | VITRDENEKLEYYEKFQIIENVFKQKKKPTLKQIAKEILVNE | |
| | | EDIKGYRVTSTGKPEFTNLKVYHDIKDITARKEIIENAELLD | |
| | | QIAKILTIYQSSEDIQEELTNLNSELTQEEIEQISNLKGYTG | |
| | | THNLSLKAINLILDELWHTNDNQIAIFNRLKLVPKKVDLSQQ | |
| | | KEIPTTLVDDFILSPVVKRSFIQSIKVINAIIKKYGLPNDII | |
| | | IELAREKNSKDAQKMINEMQKRNRQTNERIEEIIRTTGKENA | |
| | | KYLIEKIKLHDMQEGKCLYSLEAIPLEDLLNNPFNYEVDHII | |
| | | PRSVSFDNSFNNKVLVKQEENSKKGNRTPFQYLSSSDSKISY | |
| | | ETFKKHILNLAKGKGRISKTKKEYLLEERDINRFSVQKDFIN | |
| | | RNLVDTRYATRGLMNLLRSYFRVNNLDVKVKSINGGFTSELR | |
| | | RKWKFKKERNKGYKHHAEDALIIANADFIFKEWKKLDKAKKV | |
| | | MENQMFEEKIETEQEYKEIFITPHQIKHIKDFKDYKYSHRVD | |
| | | KKPNRELINDTLYSTRKDDKGNTLIVNNLNGLYDKDNDKLKK | |
| | | LINKSPEKLLMYHHDPQTYQKLKLIMEQYGDEKNPLYKYYEE | |
| | | TGNYLTKYSKKDNGPVIKKIKYYGNKLNAHLDITDDYPNSRN | |
| | | KVVKLSLKPYRFDVYLDNGVYKFVTVKNLDVIKKENYYEVNS | |
| | | KCYEEAKKLKKISNQAEFIASFYNNDLIKINGELYRVIGVNN | |
| | | DLLNRIEVNMIDITYREYLENMNDKRPPRIIKTIASKTQSIK | |
| | | KYSTDILGNLYEVKSKKHPQIIKKG | |
| pBE0042 | 30 | SEVEFSHEYWMRHALTLAKRARDEREVPVGAVLVLNNRVIGE | 10-30% |
| | | GWNRAIGLHDPTAHAEIMALRQGGLVMQNYRLIDATLYVTFE | (A8) |
| | | PCVMCAGAMIHSRIGRVVFGVRNSKRGAAGSLMNVLNYPGMN | |
| | | HRVEITEGILADECAALLCDFYRMPRQVFNAQKKAQSSINSG | |
| | | GSSGGSSGSETPGTSESATPESSGGSSGGSKRNYILGLAIGI | |
| | | TSVGYGIIDYETRDVIDAGVRLFKEANVENNEGRRSKRGARR | |
| | | LKRRRRHRIQRVKKLLFDYNLLTDHSELSGINPYEARVKGLS | |
| | | QKLSEEEFSAALLHLAKRRGVHNVNEVEEDTGNELSTKEQIS | |
| | | RNSKALEEKYVAELQLERLKKDGEVRGSINRFKTSDYVKEAK | |
| | | QLLKVQKAYHQLDQSFIDTYIDLLETRRTYYEGPGEGSPFGW | |
| | | KDIKEWYEMLMGHCTYFPEELRSVKYAYNADLYNALNDLNNL | |
| | | VITRDENEKLEYYEKFQIIENVFKQKKKPTLKQIAKEILVNE | |
| | | EDIKGYRVTSTGKPEFTNLKVYHDIKDITARKEIIENAELLD | |
| | | QIAKILTIYQSSEDIQEELTNLNSELTQEEIEQISNLKGYTG | |
| | | THNLSLKAINLILDELWHTNDNQIAIFNRLKLVPKKVDLSQQ | |
| | | KEIPTTLVDDFILSPVVKRSFIQSIKVINAIIKKYGLPNDII | |
| | | IELAREKNSKDAQKMINEMQKRNRQTNERIEEIIRTTGKENA | |
| | | KYLIEKIKLHDMQEGKCLYSLEAIPLEDLLNNPFNYEVDHII | |
| | | PRSVSFDNSFNNKVLVKQEENSKKGNRTPFQYLSSSDSKISY | |
| | | ETFKKHILNLAKGKGRISKTKKEYLLEERDINRFSVQKDFIN | |
| | | RNLVDTRYATRGLMNLLRSYFRVNNLDVKVKSINGGFTSFLR | |
| | | RKWKFKKERNKGYKHHAEDALIIANADFIFKEWKKLDKAKKV | |
| | | MENQMFEEKQAESMPEIETEQEYKEIFITPHQIKHIKDEKDY | |
| | | KYSHRVDKKPNRELINDTLYSTRKDDKGNTLIVNNLNGLYDK | |
| | | DNDKLKKLINKSPEKLLMYHHDPQTYQKLKLIMEQYGDEKNP | |
| | | LYKYYEETGNYLTKYSKKDNGPVIKKIKYYGNKLNAHLDITD | |
| | | DYPNSRNKVVKLSLKPYRFDVYLDNGVYKFVTVKNLDVIKKE | |
| | | NYYEVNSKCYEEAKKLKKISNQAEFIASFYNNDLIKINGELY | |
| | | RVIGVNNDLLNRIEVNMIDITYREYLENMNDKRPPRIIKTIA | |
| | | SKTQSIKKYSTDILGNLY | |
| pBE0043 | 31 | SEVEFSHEYWMRHALTLAKRARDEREVPVGAVLVLNNRVIGE | >90% |
| | | GWNRAIGLHDPTAHAEIMALRQGGLVMQNYRLIDATLYVTFE | (A8) |
| | | PCVMCAGAMIHSRIGRVVFGVRNSKRGAAGSLMNVLNYPGMN | |
| | | HRVEITEGILADECAALLCDFYRMPRQVFNAQKKAQSSINSG | |
| | | GSSGGSSGSETPGTSESATPESSGGSSGGSILGLAIGITSVG | |
| | | YGIIDYETRDVIDAGVRLFKEANVENNEGRRSKRGARRLKRR | |
| | | RRHRIQRVKKLLFDYNLLTDHSELSGINPYEARVKGLSQKLS | |
| | | EEEFSAALLHLAKRRGVHNVNEVEEDTGNELSTKEQISRNSK | |
| | | ALEEKYVAELQLERLKKDGEVRGSINRFKTSDYVKEAKQLLK | |
| | | VQKAYHQLDQSFIDTYIDLLETRRTYYEGPGEGSPFGWKDIK | |
| | | EWYEMLMGHCTYFPEELRSVKYAYNADLYNALNDLNNLVITR | |
| | | DENEKLEYYEKFQIIENVFKQKKKPTLKQIAKEILVNEEDIK | |
| | | GYRVTSTGKPEFTNLKVYHDIKDITARKEIIENAELLDQIAK | |
| | | ILTIYQSSEDIQEELTNLNSELTQEEIEQISNLKGYTGTHNL | |
| | | SLKAINLILDELWHTNDNQIAIFNRLKLVPKKVDLSQQKEIP | |
| | | TTLVDDFILSPVVKRSFIQSIKVINAIIKKYGLPNDIIIELA | |
| | | REKNSKDAQKMINEMQKRNRQTNERIEEIIRTTGKENAKYLI | |
| | | EKIKLHDMQEGKCLYSLEAIPLEDLLNNPFNYEVDHIIPRSV | |
| | | SFDNSFNNKVLVKQEENSKKGNRTPFQYLSSSDSKISYETFK | |
| | | KHILNLAKGKGRISKTKKEYLLEERDINRFSVQKDFINRNLV | |
| | | DTRYATRGLMNLLRSYFRVNNLDVKVKSINGGFTSFLRRKWK | |
| | | FKKERNKGYKHHAEDALIIANADFIFKEWKKLDKAKKVMENQ | |
| | | MFEEKQAESMPEIETEQEYKEIFITPHQIKHIKDFKDYKYSH | |
| | | RVDKKPNRELINDTLYSTRKDDKGNTLIVNNLNGLYDKDNDK | |
| | | LKKLINKSPEKLLMYHHDPQTYQKLKLIMEQYGDEKNPLYKY | |
| | | YEETGNYLTKYSKKDNGPVIKKIKYYGNKLNAHLDITDDYPN | |
| | | SRNKVVKLSLKPYRFDVYLDNGVYKFVTVKNLDVIKKENYYE | |
| | | VNSKCYEEAKKLKKISNQAEFIASFYNNDLIKINGELYRVIG | |
| | | VNNDLLNRIEVNMIDITYREYLENMNDKRPPRIIKTIASKTQ | |
| | | SIKKYSTDILGNLYEVKSKKHPQIIKKG | |
| pBE0060 | 32 | FSHEYWMRHALTLAKRARDEREVPVGAVLVLNNRVIGEGWNR | >90% |
| | | AIGLHDPTAHAEIMALRQGGLVMQNYRLIDATLYVTFEPCVM | (A8) |
| | | CAGAMIHSRIGRVVFGVRNSKRGAAGSLMNVLNYPGMNHRVE | |
| | | ITEGILADECAALLCDFYRMPRQVFNAQKKAQPAPAPKRNYI | |
| | | LGLAIGITSVGYGIIDYETRDVIDAGVRLFKEANVENNEGRR | |
| | | SKRGARRLKRRRRHRIQRVKKLLFDYNLLTDHSELSGINPYE | |
| | | ARVKGLSQKLSEEEFSAALLHLAKRRGVHNVNEVEEDTGNEL | |
| | | STKEQISRNSKALEEKYVAELQLERLKKDGEVRGSINRFKTS | |
| | | DYVKEAKQLLKVQKAYHQLDQSFIDTYIDLLETRRTYYEGPG | |
| | | EGSPFGWKDIKEWYEMLMGHCTYFPEELRSVKYAYNADLYNA | |
| | | LNDLNNLVITRDENEKLEYYEKFQIIENVFKQKKKPTLKQIA | |
| | | KEILVNEEDIKGYRVTSTGKPEFTNLKVYHDIKDITARKEII | |
| | | ENAELLDQIAKILTIYQSSEDIQEELTNLNSELTQEEIEQIS | |
| | | NLKGYTGTHNLSLKAINLILDELWHTNDNQIAIFNRLKLVPK | |
| | | KVDLSQQKEIPTTLVDDFILSPVVKRSFIQSIKVINAIIKKY | |
| | | GLPNDIIIELAREKNSKDAQKMINEMQKRNRQTNERIEEIIR | |
| | | TTGKENAKYLIEKIKLHDMQEGKCLYSLEAIPLEDLLNNPEN | |
| | | YEVDHIIPRSVSFDNSFNNKVLVKQEENSKKGNRTPFQYLSS | |
| | | SDSKISYETFKKHILNLAKGKGRISKTKKEYLLEERDINRES | |
| | | VQKDFINRNLVDTRYATRGLMNLLRSYFRVNNLDVKVKSING | |
| | | GFTSFLRRKWKFKKERNKGYKHHAEDALIIANADFIFKEWKK | |
| | | LDKAKKVMENQMFEEKQAESMPEIETEQEYKEIFITPHQIKH | |
| | | IKDFKDYKYSHRVDKKPNRELINDTLYSTRKDDKGNTLIVNN | |
| | | LNGLYDKDNDKLKKLINKSPEKLLMYHHDPQTYQKLKLIMEQ | |
| | | YGDEKNPLYKYYEETGNYLTKYSKKDNGPVIKKIKYYGNKLN | |
| | | AHLDITDDYPNSRNKVVKLSLKPYRFDVYLDNGVYKFVTVKN | |
| | | LDVIKKENYYEVNSKCYEEAKKLKKISNQAEFIASFYNNDLI | |
| | | KINGELYRVIGVNNDLLNRIEVNMIDITYREYLENMNDKRPP | |
| | | RIIKTIASKTQSIKKYSTDILGNLYEVKSKKHPQIIKKG | |
| pBE0061 | 33 | FSHEYWMRHALTLAKRARDEREVPVGAVLVLNNRVIGEGWNR | >90% |
| | | AIGLHDPTAHAEIMALRQGGLVMQNYRLIDATLYVTFEPCVM | (A8) |
| | | CAGAMIHSRIGRVVFGVRNSKRGAAGSLMNVLNYPGMNHRVE | |
| | | ITEGILADECAALLCDFYRMPRQVFNAQKKAQPAPAPKRNYI | |
| | | LGLAIGITSVGYGIIDYETRDVIDAGVRLFKEANVENNEGRR | |
| | | SKRGARRLKRRRRHRIQRVKKLLFDYNLLTDHSELSGINPYE | |
| | | ARVKGLSQKLSEEEFSAALLHLAKRRGVHNVNEVEEDTGNEL | |
| | | STKEQISRNSKALEEKYVAELQLERLKKDGEVRGSINRFKTS | |
| | | DYVKEAKQLLKVQKAYHQLDQSFIDTYIDLLETRRTYYEGPG | |
| | | EGSPFGWKDIKEWYEMLMGHCTYFPEELRSVKYAYNADLYNA | |
| | | LNDLNNLVITRDENEKLEYYEKFQIIENVFKQKKKPTLKQIA | |
| | | KEILVNEEDIKGYRVTSTGKPEFTNLKVYHDIKDITARKEII | |
| | | ENAELLDQIAKILTIYQSSEDIQEELTNLNSELTQEEIEQIS | |
| | | NLKGYTGTHNLSLKAINLILDELWHTNDNQIAIFNRLKLVPK | |
| | | KVDLSQQKEIPTTLVDDFILSPVVKRSFIQSIKVINAIIKKY | |
| | | GLPNDIIIELAREKNSKDAQKMINEMQKRNRQTNERIEEIIR | |
| | | TTGKENAKYLIEKIKLHDMQEGKCLYSLEAIPLEDLLNNPEN | |
| | | YEVDHIIPRSVSFDNSFNNKVLVKQEENSKKGNRTPFQYLSS | |
| | | SDSKISYETFKKHILNLAKGKGRISKTKKEYLLEERDINRES | |
| | | VQKDFINRNLVDTRYATRGLMNLLRSYFRVNNLDVKVKSING | |
| | | GFTSFLRRKFKKERNKGYKHHAEDALIIANADFIFKDKAKKV | |
| | | MENQMFEEKQAESMPEIETEQEYKEIFITPHQIKHIKDEKDY | |
| | | KYSHRVDKKPNRELINDTLYSTRKDDKGNTLIVNNLNGLYDK | |
| | | DNDKLKKLINKSPEKLLMYHHDPQTYQKLKLIMEQYGDEKNP | |
| | | LYKYYEETGNYLTKYSKKDNGPVIKKIKYYGNKLNAHLDITD | |
| | | DYPNSRNKVVKLSLKPYRFDVYLDNGVYKFVTVKNLDVIKKE | |
| | | NYYEVNSKCYEEAKKLKKISNQAEFIASFYNNDLIKINGELY | |
| | | RVIGVNNDLLNRIEVNMIDITYREYLENMNDKRPPRIIKTIA | |
| | | SKTQSIKKYSTDILGNLYEVKSKKHPQIIKKG | |
| pBE0105 | 34 | FSHEYWMRHALTLAKRARDEREVPVGAVLVLNNRVIGEGWNR | >90% |
| | | AIGLHDPTAHAEIMALRQGGLVMQNYRLIDATLYVTFEPCVM | (A5) |
| | | CAGAMIHSRIGRVVFGVRNSKRGAAGSLMNVLNYPGMNHRVE | |
| | | ITEGILADECAALLCDFYRMPRQVFNAQPAPAPILGLAIGIT | |
| | | SVGYGIIDYETRDVIDAGVRLFKEANVENNEGRRSKRGARRL | |
| | | KRRRRHRIQRVKKLLFDYNLLTDHSELSGINPYEARVKGLSQ | |
| | | KLSEEEFSAALLHLAKRRGVHNVNEVEEDTGNELSTKEQISR | |
| | | NSKALEEKYVAELQLERLKKDGEVRGSINRFKTSDYVKEAKQ | |
| | | LLKVQKAYHQLDQSFIDTYIDLLETRRTYYEGPGEGSPFGWK | |
| | | DIKEWYEMLMGHCTYFPEELRSVKYAYNADLYNALNDLNNLV | |
| | | ITRDENEKLEYYEKFQIIENVFKQKKKPTLKQIAKEILVNEE | |
| | | DIKGYRVTSTGKPEFTNLKVYHDIKDITARKEIIENAELLDQ | |
| | | IAKILTIYQSSEDIQEELTNLNSELTQEEIEQISNLKGYTGT | |
| | | HNLSLKAINLILDELWHTNDNQIAIFNRLKLVPKKVDLSQQK | |
| | | EIPTTLVDDFILSPVVKRSFIQSIKVINAIIKKYGLPNDIII | |
| | | ELAREKNSKDAQKMINEMQKRNRQTNERIEEIIRTTGKENAK | |
| | | YLIEKIKLHDMQEGKCLYSLEAIPLEDLLNNPFNYEVDHIIP | |
| | | RSVSFDNSFNNKVLVKQEENSKKGNRTPFQYLSSSDSKISYE | |
| | | TFKKHILNLAKGKGRISKTKKEYLLEERDINRFSVQKDFINR | |
| | | NLVDTRYATRGLMNLLRSYFRVNNLDVKVKSINGGFTSFLRR | |
| | | KWKFKKERNKGYKHHAEDALIIANADFIFKEWKKLDKAKKVM | |
| | | ENQMFEEKQAESMPEIETEQEYKEIFITPHQIKHIKDFKDYK | |
| | | YSHRVDKKPNRELINDTLYSTRKDDKGNTLIVNNLNGLYDKD | |
| | | NDKLKKLINKSPEKLLMYHHDPQTYQKLKLIMEQYGDEKNPL | |
| | | YKYYEETGNYLTKYSKKDNGPVIKKIKYYGNKLNAHLDITDD | |
| | | YPNSRNKVVKLSLKPYRFDVYLDNGVYKFVTVKNLDVIKKEN | |
| | | YYEVNSKCYEEAKKLKKISNQAEFIASFYNNDLIKINGELYR | |
| | | VIGVNNDLLNRIEVNMIDITYREYLENMNDKRPPRIIKTIAS | |
| | | KTQSIKKYSTDILGNLYEVKSKKHPQIIKKG | |
| pBE0107 | 35 | YWMRHALTLAKRARDEREVPVGAVLVLNNRVIGEGWNRAIGL | >90% |
| | | HDPTAHAEIMALRQGGLVMQNYRLIDATLYVTFEPCVMCAGA | (A5) |
| | | MIHSRIGRVVFGVRNSKRGAAGSLMNVLNYPGMNHRVEITEG | |
| | | ILADECAALLCDFYRMPRQVFNAQKKAQPAPAPILGLAIGIT | |
| | | SVGYGIIDYETRDVIDAGVRLFKEANVENNEGRRSKRGARRL | |
| | | KRRRRHRIQRVKKLLFDYNLLTDHSELSGINPYEARVKGLSQ | |
| | | KLSEEEFSAALLHLAKRRGVHNVNEVEEDTGNELSTKEQISR | |
| | | NSKALEEKYVAELQLERLKKDGEVRGSINRFKTSDYVKEAKQ | |
| | | LLKVQKAYHQLDQSFIDTYIDLLETRRTYYEGPGEGSPFGWK | |
| | | DIKEWYEMLMGHCTYFPEELRSVKYAYNADLYNALNDLNNLV | |
| | | ITRDENEKLEYYEKFQIIENVFKQKKKPTLKQIAKEILVNEE | |
| | | DIKGYRVTSTGKPEFTNLKVYHDIKDITARKEIIENAELLDQ | |
| | | IAKILTIYQSSEDIQEELTNLNSELTQEEIEQISNLKGYTGT | |
| | | HNLSLKAINLILDELWHTNDNQIAIFNRLKLVPKKVDLSQQK | |
| | | EIPTTLVDDFILSPVVKRSFIQSIKVINAIIKKYGLPNDIII | |
| | | ELAREKNSKDAQKMINEMQKRNRQTNERIEEIIRTTGKENAK | |
| | | YLIEKIKLHDMQEGKCLYSLEAIPLEDLLNNPFNYEVDHIIP | |
| | | RSVSFDNSFNNKVLVKQEENSKKGNRTPFQYLSSSDSKISYE | |
| | | TFKKHILNLAKGKGRISKTKKEYLLEERDINRFSVQKDFINR | |
| | | NLVDTRYATRGLMNLLRSYFRVNNLDVKVKSINGGFTSFLRR | |
| | | KWKFKKERNKGYKHHAEDALIIANADFIFKEWKKLDKAKKVM | |
| | | ENQMFEEKQAESMPEIETEQEYKEIFITPHQIKHIKDFKDYK | |
| | | YSHRVDKKPNRELINDTLYSTRKDDKGNTLIVNNLNGLYDKD | |
| | | NDKLKKLINKSPEKLLMYHHDPQTYQKLKLIMEQYGDEKNPL | |
| | | YKYYEETGNYLTKYSKKDNGPVIKKIKYYGNKLNAHLDITDD | |
| | | YPNSRNKVVKLSLKPYRFDVYLDNGVYKFVTVKNLDVIKKEN | |
| | | YYEVNSKCYEEAKKLKKISNQAEFIASFYNNDLIKINGELYR | |
| | | VIGVNNDLLNRIEVNMIDITYREYLENMNDKRPPRIIKTIAS | |
| | | KTQSIKKYSTDILGNLYEVKSKKHPQIIKKG | |
| pBE0108 | 36 | YWMRHALTLAKRARDEREVPVGAVLVLNNRVIGEGWNRAIGL | >90% |
| | | HDPTAHAEIMALRQGGLVMQNYRLIDATLYVTFEPCVMCAGA | (A5) |
| | | MIHSRIGRVVFGVRNSKRGAAGSLMNVLNYPGMNHRVEITEG | |
| | | ILADECAALLCDFYRMPRQVFNAQKKPAPAPKRNYILGLAIG | |
| | | ITSVGYGIIDYETRDVIDAGVRLFKEANVENNEGRRSKRGAR | |
| | | RLKRRRRHRIQRVKKLLFDYNLLTDHSELSGINPYEARVKGL | |
| | | SQKLSEEEFSAALLHLAKRRGVHNVNEVEEDTGNELSTKEQI | |
| | | SRNSKALEEKYVAELQLERLKKDGEVRGSINRFKTSDYVKEA | |
| | | KQLLKVQKAYHQLDQSFIDTYIDLLETRRTYYEGPGEGSPFG | |
| | | WKDIKEWYEMLMGHCTYFPEELRSVKYAYNADLYNALNDLNN | |
| | | LVITRDENEKLEYYEKFQIIENVFKQKKKPTLKQIAKEILVN | |
| | | EEDIKGYRVTSTGKPEFTNLKVYHDIKDITARKEIIENAELL | |
| | | DQIAKILTIYQSSEDIQEELTNLNSELTQEEIEQISNLKGYT | |
| | | GTHNLSLKAINLILDELWHTNDNQIAIFNRLKLVPKKVDLSQ | |
| | | QKEIPTTLVDDFILSPVVKRSFIQSIKVINAIIKKYGLPNDI | |
| | | IIELAREKNSKDAQKMINEMQKRNRQTNERIEEIIRTTGKEN | |
| | | AKYLIEKIKLHDMQEGKCLYSLEAIPLEDLLNNPFNYEVDHI | |
| | | IPRSVSFDNSFNNKVLVKQEENSKKGNRTPFQYLSSSDSKIS | |
| | | YETFKKHILNLAKGKGRISKTKKEYLLEERDINRFSVQKDFI | |
| | | NRNLVDTRYATRGLMNLLRSYFRVNNLDVKVKSINGGFTSEL | |
| | | RRKWKFKKERNKGYKHHAEDALIIANADFIFKEWKKLDKAKK | |
| | | VMENQMFEEKQAESMPEIETEQEYKEIFITPHQIKHIKDEKD | |
| | | YKYSHRVDKKPNRELINDTLYSTRKDDKGNTLIVNNLNGLYD | |
| | | KDNDKLKKLINKSPEKLLMYHHDPQTYQKLKLIMEQYGDEKN | |
| | | PLYKYYEETGNYLTKYSKKDNGPVIKKIKYYGNKLNAHLDIT | |
| | | DDYPNSRNKVVKLSLKPYRFDVYLDNGVYKFVTVKNLDVIKK | |
| | | ENYYEVNSKCYEEAKKLKKISNQAEFIASFYNNDLIKINGEL | |
| | | YRVIGVNNDLLNRIEVNMIDITYREYLENMNDKRPPRIIKTI | |
| | | ASKTQSIKKYSTDILGNLYEVKSKKHPQIIKKG | |
| pBE0109 | 37 | YWMRHALTLAKRARDEREVPVGAVLVLNNRVIGEGWNRAIGL | >90% |
| | | HDPTAHAEIMALRQGGLVMQNYRLIDATLYVTFEPCVMCAGA | (A5) |
| | | MIHSRIGRVVFGVRNSKRGAAGSLMNVLNYPGMNHRVEITEG | |
| | | ILADECAALLCDFYRMPRQVFNAQKKPAPAPILGLAIGITSV | |
| | | GYGIIDYETRDVIDAGVRLFKEANVENNEGRRSKRGARRLKR | |
| | | RRRHRIQRVKKLLFDYNLLTDHSELSGINPYEARVKGLSQKL | |
| | | SEEEFSAALLHLAKRRGVHNVNEVEEDTGNELSTKEQISRNS | |
| | | KALEEKYVAELQLERLKKDGEVRGSINRFKTSDYVKEAKQLL | |
| | | KVQKAYHQLDQSFIDTYIDLLETRRTYYEGPGEGSPFGWKDI | |
| | | KEWYEMLMGHCTYFPEELRSVKYAYNADLYNALNDLNNLVIT | |
| | | RDENEKLEYYEKFQIIENVFKQKKKPTLKQIAKEILVNEEDI | |
| | | KGYRVTSTGKPEFTNLKVYHDIKDITARKEIIENAELLDQIA | |
| | | KILTIYQSSEDIQEELTNLNSELTQEEIEQISNLKGYTGTHN | |
| | | LSLKAINLILDELWHTNDNQIAIFNRLKLVPKKVDLSQQKEI | |
| | | PTTLVDDFILSPVVKRSFIQSIKVINAIIKKYGLPNDIIIEL | |
| | | AREKNSKDAQKMINEMQKRNRQTNERIEEIIRTTGKENAKYL | |
| | | IEKIKLHDMQEGKCLYSLEAIPLEDLLNNPFNYEVDHIIPRS | |
| | | VSFDNSFNNKVLVKQEENSKKGNRTPFQYLSSSDSKISYETF | |
| | | KKHILNLAKGKGRISKTKKEYLLEERDINRFSVQKDFINRNL | |
| | | VDTRYATRGLMNLLRSYFRVNNLDVKVKSINGGFTSFLRRKW | |
| | | KFKKERNKGYKHHAEDALIIANADFIFKEWKKLDKAKKVMEN | |
| | | QMFEEKQAESMPEIETEQEYKEIFITPHQIKHIKDFKDYKYS | |
| | | HRVDKKPNRELINDTLYSTRKDDKGNTLIVNNLNGLYDKDND | |
| | | KLKKLINKSPEKLLMYHHDPQTYQKLKLIMEQYGDEKNPLYK | |
| | | YYEETGNYLTKYSKKDNGPVIKKIKYYGNKLNAHLDITDDYP | |
| | | NSRNKVVKLSLKPYRFDVYLDNGVYKFVTVKNLDVIKKENYY | |
| | | EVNSKCYEEAKKLKKISNQAEFIASFYNNDLIKINGELYRVI | |
| | | GVNNDLLNRIEVNMIDITYREYLENMNDKRPPRIIKTIASKT | |
| | | QSIKKYSTDILGNLYEVKSKKHPQIIKKG | |
| pBE0110 | 38 | YWMRHALTLAKRARDEREVPVGAVLVLNNRVIGEGWNRAIGL | 50-75% |
| | | HDPTAHAEIMALRQGGLVMQNYRLIDATLYVTFEPCVMCAGA | (A8) |
| | | MIHSRIGRVVFGVRNSKRGAAGSLMNVLNYPGMNHRVEITEG | |
| | | ILADECAALLCDFYRMPRQVFNAQPAPAPKRNYILGLAIGIT | |
| | | SVGYGIIDYETRDVIDAGVRLFKEANVENNEGRRSKRGARRL | |
| | | KRRRRHRIQRVKKLLFDYNLLTDHSELSGINPYEARVKGLSQ | |
| | | KLSEEEFSAALLHLAKRRGVHNVNEVEEDTGNELSTKEQISR | |
| | | NSKALEEKYVAELQLERLKKDGEVRGSINRFKTSDYVKEAKQ | |
| | | LLKVQKAYHQLDQSFIDTYIDLLETRRTYYEGPGEGSPFGWK | |
| | | DIKEWYEMLMGHCTYFPEELRSVKYAYNADLYNALNDLNNLV | |
| | | ITRDENEKLEYYEKFQIIENVFKQKKKPTLKQIAKEILVNEE | |
| | | DIKGYRVTSTGKPEFTNLKVYHDIKDITARKEIIENAELLDQ | |
| | | IAKILTIYQSSEDIQEELTNLNSELTQEEIEQISNLKGYTGT | |
| | | HNLSLKAINLILDELWHTNDNQIAIFNRLKLVPKKVDLSQQK | |
| | | EIPTTLVDDFILSPVVKRSFIQSIKVINAIIKKYGLPNDIII | |
| | | ELAREKNSKDAQKMINEMQKRNRQTNERIEEIIRTTGKENAK | |
| | | YLIEKIKLHDMQEGKCLYSLEAIPLEDLLNNPFNYEVDHIIP | |
| | | RSVSFDNSFNNKVLVKQEENSKKGNRTPFQYLSSSDSKISYE | |
| | | TFKKHILNLAKGKGRISKTKKEYLLEERDINRFSVQKDFINR | |
| | | NLVDTRYATRGLMNLLRSYFRVNNLDVKVKSINGGFTSFLRR | |
| | | KWKFKKERNKGYKHHAEDALIIANADFIFKEWKKLDKAKKVM | |
| | | ENQMFEEKQAESMPEIETEQEYKEIFITPHQIKHIKDFKDYK | |
| | | YSHRVDKKPNRELINDTLYSTRKDDKGNTLIVNNLNGLYDKD | |
| | | NDKLKKLINKSPEKLLMYHHDPQTYQKLKLIMEQYGDEKNPL | |
| | | YKYYEETGNYLTKYSKKDNGPVIKKIKYYGNKLNAHLDITDD | |
| | | YPNSRNKVVKLSLKPYRFDVYLDNGVYKFVTVKNLDVIKKEN | |
| | | YYEVNSKCYEEAKKLKKISNQAEFIASFYNNDLIKINGELYR | |
| | | VIGVNNDLLNRIEVNMIDITYREYLENMNDKRPPRIIKTIAS | |
| | | KTQSIKKYSTDILGNLYEVKSKKHPQIIKKG | |
| pBE0115 | 63 | MRHALTLAKRARDEREVPVGAVLVLNNRVIGEGWNRAIGLHD | <10% |
| | | PTAHAEIMALRQGGLVMQNYRLIDATLYVTFEPCVMCAGAMI | (A8) |
| | | HSRIGRVVFGVRNSKRGAAGSLMNVLNYPGMNHRVEITEGIL | |
| | | ADECAALLCDFYRMPRQVFNAQKKAQSSINSGGSSGGSSGSE | |
| | | TPGTSESATPESSGGSSGGSKRNYILGLAIGITSVGYGIIDY | |
| | | ETRDVIDAGVRLFKEANVENNEGRRSKRGARRLKRRRRHRIQ | |
| | | RVKKLLFDYNLLTDHSELSGINPYEARVKGLSQKLSEEEFSA | |
| | | ALLHLAKRRGVHNVNEVEEDTGNELSTKEQISRNSKALEEKY | |
| | | VAELQLERLKKDGEVRGSINRFKTSDYVKEAKQLLKVQKAYH | |
| | | QLDQSFIDTYIDLLETRRTYYEGPGEGSPFGWKDIKEWYEML | |
| | | MGHCTYFPEELRSVKYAYNADLYNALNDLNNLVITRDENEKL | |
| | | EYYEKFQIIENVFKQKKKPTLKQIAKEILVNEEDIKGYRVTS | |
| | | TGKPEFTNLKVYHDIKDITARKEIIENAELLDQIAKILTIYQ | |
| | | SSEDIQEELTNLNSELTQEEIEQISNLKGYTGTHNLSLKAIN | |
| | | LILDELWHTNDNQIAIFNRLKLVPKKVDLSQQKEIPTTLVDD | |
| | | FILSPVVKRSFIQSIKVINAIIKKYGLPNDIIIELAREKNSK | |
| | | DAQKMINEMQKRNRQTNERIEEIIRTTGKENAKYLIEKIKLH | |
| | | DMQEGKCLYSLEAIPLEDLLNNPFNYEVDHIIPRSVSEDNSF | |
| | | NNKVLVKQEENSKKGNRTPFQYLSSSDSKISYETFKKHILNL | |
| | | AKGKGRISKTKKEYLLEERDINRFSVQKDFINRNLVDTRYAT | |
| | | RGLMNLLRSYFRVNNLDVKVKSINGGFTSFLRRKWKFKKERN | |
| | | KGYKHHAEDALIIANADFIFKEWKKLDKAKKVMENQMFEEKQ | |
| | | AESMPEIETEQEYKEIFITPHQIKHIKDFKDYKYSHRVDKKP | |
| | | NRELINDTLYSTRKDDKGNTLIVNNLNGLYDKDNDKLKKLIN | |
| | | KSPEKLLMYHHDPQTYQKLKLIMEQYGDEKNPLYKYYEETGN | |
| | | YLTKYSKKDNGPVIKKIKYYGNKLNAHLDITDDYPNSRNKVV | |
| | | KLSLKPYRFDVYLDNGVYKFVTVKNLDVIKKENYYEVNSKCY | |
| | | EEAKKLKKISNQAEFIASFYNNDLIKINGELYRVIGVNNDLL | |
| | | NRIEVNMIDITYREYLENMNDKRPPRIIKTIASKTQSIKKYS | |
| | | TDILGNLYEVKSKKHPQIIKKG | |
| pBE0116 | 39 | HALTLAKRARDEREVPVGAVLVLNNRVIGEGWNRAIGLHDPT | Repeat |
| | | AHAEIMALRQGGLVMQNYRLIDATLYVTFEPCVMCAGAMIHS | required |
| | | RIGRVVFGVRNSKRGAAGSLMNVLNYPGMNHRVEITEGILAD | |
| | | ECAALLCDFYRMPRQVFNAQKKAQSSINSGGSSGGSSGSETP | |
| | | GTSESATPESSGGSSGGSKRNYILGLAIGITSVGYGIIDYET | |
| | | RDVIDAGVRLFKEANVENNEGRRSKRGARRLKRRRRHRIQRV | |
| | | KKLLFDYNLLTDHSELSGINPYEARVKGLSQKLSEEEFSAAL | |
| | | LHLAKRRGVHNVNEVEEDTGNELSTKEQISRNSKALEEKYVA | |
| | | ELQLERLKKDGEVRGSINRFKTSDYVKEAKQLLKVQKAYHQL | |
| | | DQSFIDTYIDLLETRRTYYEGPGEGSPFGWKDIKEWYEMLMG | |
| | | HCTYFPEELRSVKYAYNADLYNALNDLNNLVITRDENEKLEY | |
| | | YEKFQIIENVFKQKKKPTLKQIAKEILVNEEDIKGYRVTSTG | |
| | | KPEFTNLKVYHDIKDITARKEIIENAELLDQIAKILTIYQSS | |
| | | EDIQEELTNLNSELTQEEIEQISNLKGYTGTHNLSLKAINLI | |
| | | LDELWHTNDNQIAIFNRLKLVPKKVDLSQQKEIPTTLVDDFI | |
| | | LSPVVKRSFIQSIKVINAIIKKYGLPNDIIIELAREKNSKDA | |
| | | QKMINEMQKRNRQTNERIEEIIRTTGKENAKYLIEKIKLHDM | |
| | | QEGKCLYSLEAIPLEDLLNNPFNYEVDHIIPRSVSFDNSENN | |
| | | KVLVKQEENSKKGNRTPFQYLSSSDSKISYETFKKHILNLAK | |
| | | GKGRISKTKKEYLLEERDINRFSVQKDFINRNLVDTRYATRG | |
| | | LMNLLRSYFRVNNLDVKVKSINGGFTSFLRRKWKFKKERNKG | |
| | | YKHHAEDALIIANADFIFKEWKKLDKAKKVMENQMFEEKQAE | |
| | | SMPEIETEQEYKEIFITPHQIKHIKDFKDYKYSHRVDKKPNR | |
| | | ELINDTLYSTRKDDKGNTLIVNNLNGLYDKDNDKLKKLINKS | |
| | | PEKLLMYHHDPQTYQKLKLIMEQYGDEKNPLYKYYEETGNYL | |
| | | TKYSKKDNGPVIKKIKYYGNKLNAHLDITDDYPNSRNKVVKL | |
| | | SLKPYRFDVYLDNGVYKFVTVKNLDVIKKENYYEVNSKCYEE | |
| | | AKKLKKISNQAEFIASFYNNDLIKINGELYRVIGVNNDLLNR | |
| | | IEVNMIDITYREYLENMNDKRPPRIIKTIASKTQSIKKYSTD | |
| | | ILGNLYEVKSKKHPQIIKKG | |
| pBE0117 | 40 | SEVEFSHEYWMRHALTLAKRARDEREVPVGAVLVLNNRVIGE | 50-75% |
| | | GWNRAIGLHDPTAHAEIMALRQGGLVMQNYRLIDATLYVTFE | (A8) |
| | | PCVMCAGAMIHSRIGRVVFGVRNSKRGAAGSLMNVLNYPGMN | |
| | | HRVEITEGILADECAALLCDFYRMPRQVFNSGGSSGGSSGSE | |
| | | TPGTSESATPESSGGSSGGSKRNYILGLAIGITSVGYGIIDY | |
| | | ETRDVIDAGVRLFKEANVENNEGRRSKRGARRLKRRRRHRIQ | |
| | | RVKKLLFDYNLLTDHSELSGINPYEARVKGLSQKLSEEEFSA | |
| | | ALLHLAKRRGVHNVNEVEEDTGNELSTKEQISRNSKALEEKY | |
| | | VAELQLERLKKDGEVRGSINRFKTSDYVKEAKQLLKVQKAYH | |
| | | QLDQSFIDTYIDLLETRRTYYEGPGEGSPFGWKDIKEWYEML | |
| | | MGHCTYFPEELRSVKYAYNADLYNALNDLNNLVITRDENEKL | |
| | | EYYEKFQIIENVFKQKKKPTLKQIAKEILVNEEDIKGYRVTS | |
| | | TGKPEFTNLKVYHDIKDITARKEIIENAELLDQIAKILTIYQ | |
| | | SSEDIQEELTNLNSELTQEEIEQISNLKGYTGTHNLSLKAIN | |
| | | LILDELWHTNDNQIAIFNRLKLVPKKVDLSQQKEIPTTLVDD | |
| | | FILSPVVKRSFIQSIKVINAIIKKYGLPNDIIIELAREKNSK | |
| | | DAQKMINEMQKRNRQTNERIEEIIRTTGKENAKYLIEKIKLH | |
| | | DMQEGKCLYSLEAIPLEDLLNNPFNYEVDHIIPRSVSEDNSF | |
| | | NNKVLVKQEENSKKGNRTPFQYLSSSDSKISYETFKKHILNL | |
| | | AKGKGRISKTKKEYLLEERDINRFSVQKDFINRNLVDTRYAT | |
| | | RGLMNLLRSYFRVNNLDVKVKSINGGFTS FLRRKWKFKKERN | |
| | | KGYKHHAEDALIIANADFIFKEWKKLDKAKKVMENQMFEEKQ | |
| | | AESMPEIETEQEYKEIFITPHQIKHIKDFKDYKYSHRVDKKP | |
| | | NRELINDTLYSTRKDDKGNTLIVNNLNGLYDKDNDKLKKLIN | |
| | | KSPEKLLMYHHDPQTYQKLKLIMEQYGDEKNPLYKYYEETGN | |
| | | YLTKYSKKDNGPVIKKIKYYGNKLNAHLDITDDYPNSRNKVV | |
| | | KLSLKPYRFDVYLDNGVYKFVTVKNLDVIKKENYYEVNSKCY | |
| | | EEAKKLKKISNQAEFIASFYNNDLIKINGELYRVIGVNNDLL | |
| | | NRIEVNMIDITYREYLENMNDKRPPRIIKTIASKTQSIKKYS | |
| | | TDILGNLYEVKSKKHPQIIKKG | |
| pBE0118 | 41 | SEVEFSHEYWMRHALTLAKRARDEREVPVGAVLVLNNRVIGE | Repeat |
| | | GWNRAIGLHDPTAHAEIMALRQGGLVMQNYRLIDATLYVTFE | Required |
| | | PCVMCAGAMIHSRIGRVVFGVRNSKRGAAGSLMNVLNYPGMN | |
| | | HRVEITEGILADECAALLCDFYRMPRQVSGGSSGGSSGSETP | |
| | | GTSESATPESSGGSSGGSKRNYILGLAIGITSVGYGIIDYET | |
| | | RDVIDAGVRLFKEANVENNEGRRSKRGARRLKRRRRHRIQRV | |
| | | KKLLFDYNLLTDHSELSGINPYEARVKGLSQKLSEEEFSAAL | |
| | | LHLAKRRGVHNVNEVEEDTGNELSTKEQISRNSKALEEKYVA | |
| | | ELQLERLKKDGEVRGSINRFKTSDYVKEAKQLLKVQKAYHQL | |
| | | DQSFIDTYIDLLETRRTYYEGPGEGSPFGWKDIKEWYEMLMG | |
| | | HCTYFPEELRSVKYAYNADLYNALNDLNNLVITRDENEKLEY | |
| | | YEKFQIIENVFKQKKKPTLKQIAKEILVNEEDIKGYRVTSTG | |
| | | KPEFTNLKVYHDIKDITARKEIIENAELLDQIAKILTIYQSS | |
| | | EDIQEELTNLNSELTQEEIEQISNLKGYTGTHNLSLKAINLI | |
| | | LDELWHTNDNQIAIFNRLKLVPKKVDLSQQKEIPTTLVDDFI | |
| | | LSPVVKRSFIQSIKVINAIIKKYGLPNDIIIELAREKNSKDA | |
| | | QKMINEMQKRNRQTNERIEEIIRTTGKENAKYLIEKIKLHDM | |
| | | QEGKCLYSLEAIPLEDLLNNPFNYEVDHIIPRSVSEDNSENN | |
| | | KVLVKQEENSKKGNRTPFQYLSSSDSKISYETFKKHILNLAK | |
| | | GKGRISKTKKEYLLEERDINRFSVQKDFINRNLVDTRYATRG | |
| | | LMNLLRSYFRVNNLDVKVKSINGGFTSFLRRKWKFKKERNKG | |
| | | YKHHAEDALIIANADFIFKEWKKLDKAKKVMENQMFEEKQAE | |
| | | SMPEIETEQEYKEIFITPHQIKHIKDFKDYKYSHRVDKKPNR | |
| | | ELINDTLYSTRKDDKGNTLIVNNLNGLYDKDNDKLKKLINKS | |
| | | PEKLLMYHHDPQTYQKLKLIMEQYGDEKNPLYKYYEETGNYL | |
| | | TKYSKKDNGPVIKKIKYYGNKLNAHLDITDDYPNSRNKVVKL | |
| | | SLKPYRFDVYLDNGVYKFVTVKNLDVIKKENYYEVNSKCYEE | |
| | | AKKLKKISNQAEFIASFYNNDLIKINGELYRVIGVNNDLLNR | |
| | | IEVNMIDITYREYLENMNDKRPPRIIKTIASKTQSIKKYSTD | |
| | | ILGNLYEVKSKKHPQIIKKG | |
| pBE0120 | 42 | SEVEFSHEYWMRHALTLAKRARDEREVPVGAVLVLNNRVIGE | >90% |
| | | GWNRAIGLHDPTAHAEIMALRQGGLVMQNYRLIDATLYVTFE | (A8) |
| | | PCVMCAGAMIHSRIGRVVFGVRNSKRGAAGSLMNVLNYPGMN | |
| | | HRVEITEGILADECAALLCDFYRMPRQVFNAQKKAQSSINSG | |
| | | GSSGGSSGSETPGTSESATPESSGGSSGGSGLAIGITSVGYG | |
| | | IIDYETRDVIDAGVRLFKEANVENNEGRRSKRGARRLKRRRR | |
| | | HRIQRVKKLLFDYNLLTDHSELSGINPYEARVKGLSQKLSEE | |
| | | EFSAALLHLAKRRGVHNVNEVEEDTGNELSTKEQISRNSKAL | |
| | | EEKYVAELQLERLKKDGEVRGSINRFKTSDYVKEAKQLLKVQ | |
| | | KAYHQLDQSFIDTYIDLLETRRTYYEGPGEGSPFGWKDIKEW | |
| | | YEMLMGHCTYFPEELRSVKYAYNADLYNALNDLNNLVITRDE | |
| | | NEKLEYYEKFQIIENVFKQKKKPTLKQIAKEILVNEEDIKGY | |
| | | RVTSTGKPEFTNLKVYHDIKDITARKEIIENAELLDQIAKIL | |
| | | TIYQSSEDIQEELTNLNSELTQEEIEQISNLKGYTGTHNLSL | |
| | | KAINLILDELWHTNDNQIAIFNRLKLVPKKVDLSQQKEIPTT | |
| | | LVDDFILSPVVKRSFIQSIKVINAIIKKYGLPNDIIIELARE | |
| | | KNSKDAQKMINEMQKRNRQTNERIEEIIRTTGKENAKYLIEK | |
| | | IKLHDMQEGKCLYSLEAIPLEDLLNNPFNYEVDHIIPRSVSF | |
| | | DNSFNNKVLVKQEENSKKGNRTPFQYLSSSDSKISYETFKKH | |
| | | ILNLAKGKGRISKTKKEYLLEERDINRFSVQKDFINRNLVDT | |
| | | RYATRGLMNLLRSYFRVNNLDVKVKSINGGFTSFLRRKWKFK | |
| | | KERNKGYKHHAEDALIIANADFIFKEWKKLDKAKKVMENQME | |
| | | EEKQAESMPEIETEQEYKEIFITPHQIKHIKDFKDYKYSHRV | |
| | | DKKPNRELINDTLYSTRKDDKGNTLIVNNLNGLYDKDNDKLK | |
| | | KLINKSPEKLLMYHHDPQTYQKLKLIMEQYGDEKNPLYKYYE | |
| | | ETGNYLTKYSKKDNGPVIKKIKYYGNKLNAHLDITDDYPNSR | |
| | | NKVVKLSLKPYRFDVYLDNGVYKFVTVKNLDVIKKENYYEVN | |
| | | SKCYEEAKKLKKISNQAEFIASFYNNDLIKINGELYRVIGVN | |
| | | NDLLNRIEVNMIDITYREYLENMNDKRPPRIIKTIASKTQSI | |
| | | KKYSTDILGNLYEVKSKKHPQIIKKG | |
| pBE0121 | 43 | SEVEFSHEYWMRHALTLAKRARDEREVPVGAVLVLNNRVIGE | 10-30% |
| | | GWNRAIGLHDPTAHAEIMALRQGGLVMQNYRLIDATLYVTFE | (A8) |
| | | PCVMCAGAMIHSRIGRVVFGVRNSKRGAAGSLMNVLNYPGMN | |
| | | HRVEITEGILADECAALLCDFYRMPRQVFNAQKKAQSSINSG | |
| | | GSSGGSSGSETPGTSESATPESSGGSSGGSAIGITSVGYGII | |
| | | DYETRDVIDAGVRLFKEANVENNEGRRSKRGARRLKRRRRHR | |
| | | IQRVKKLLFDYNLLTDHSELSGINPYEARVKGLSQKLSEEEF | |
| | | SAALLHLAKRRGVHNVNEVEEDTGNELSTKEQISRNSKALEE | |
| | | KYVAELQLERLKKDGEVRGSINRFKTSDYVKEAKQLLKVQKA | |
| | | YHQLDQSFIDTYIDLLETRRTYYEGPGEGSPFGWKDIKEWYE | |
| | | MLMGHCTYFPEELRSVKYAYNADLYNALNDLNNLVITRDENE | |
| | | KLEYYEKFQIIENVFKQKKKPTLKQIAKEILVNEEDIKGYRV | |
| | | TSTGKPEFTNLKVYHDIKDITARKEIIENAELLDQIAKILTI | |
| | | YQSSEDIQEELTNLNSELTQEEIEQISNLKGYTGTHNLSLKA | |
| | | INLILDELWHTNDNQIAIFNRLKLVPKKVDLSQQKEIPTTLV | |
| | | DDFILSPVVKRSFIQSIKVINAIIKKYGLPNDIIIELAREKN | |
| | | SKDAQKMINEMQKRNRQTNERIEEIIRTTGKENAKYLIEKIK | |
| | | LHDMQEGKCLYSLEAIPLEDLLNNPFNYEVDHIIPRSVSEDN | |
| | | SFNNKVLVKQEENSKKGNRTPFQYLSSSDSKISYETFKKHIL | |
| | | NLAKGKGRISKTKKEYLLEERDINRFSVQKDFINRNLVDTRY | |
| | | ATRGLMNLLRSYFRVNNLDVKVKSINGGFTSFLRRKWKFKKE | |
| | | RNKGYKHHAEDALIIANADFIFKEWKKLDKAKKVMENQMFEE | |
| | | KQAESMPEIETEQEYKEIFITPHQIKHIKDFKDYKYSHRVDK | |
| | | KPNRELINDTLYSTRKDDKGNTLIVNNLNGLYDKDNDKLKKL | |
| | | INKSPEKLLMYHHDPQTYQKLKLIMEQYGDEKNPLYKYYEET | |
| | | GNYLTKYSKKDNGPVIKKIKYYGNKLNAHLDITDDYPNSRNK | |
| | | VVKLSLKPYRFDVYLDNGVYKFVTVKNLDVIKKENYYEVNSK | |
| | | CYEEAKKLKKISNQAEFIASFYNNDLIKINGELYRVIGVNND | |
| | | LLNRIEVNMIDITYREYLENMNDKRPPRIIKTIASKTQSIKK | |
| | | YSTDILGNLYEVKSKKHPQIIKKG | |
| pBE0122 | 44 | SEVEFSHEYWMRHALTLAKRARDEREVPVGAVLVLNNRVIGE | <10% |
| | | GWNRAIGLHDPTAHAEIMALRQGGLVMQNYRLIDATLYVTFE | (A8) |
| | | PCVMCAGAMIHSRIGRVVFGVRNSKRGAAGSLMNVLNYPGMN | |
| | | HRVEITEGILADECAALLCDFYRMPRQVFNAQKKAQSSINSG | |
| | | GSSGGSSGSETPGTSESATPESSGGSSGGSGITSVGYGIIDY | |
| | | ETRDVIDAGVRLFKEANVENNEGRRSKRGARRLKRRRRHRIQ | |
| | | RVKKLLFDYNLLTDHSELSGINPYEARVKGLSQKLSEEEFSA | |
| | | ALLHLAKRRGVHNVNEVEEDTGNELSTKEQISRNSKALEEKY | |
| | | VAELQLERLKKDGEVRGSINRFKTSDYVKEAKQLLKVQKAYH | |
| | | QLDQSFIDTYIDLLETRRTYYEGPGEGSPFGWKDIKEWYEML | |
| | | MGHCTYFPEELRSVKYAYNADLYNALNDLNNLVITRDENEKL | |
| | | EYYEKFQIIENVFKQKKKPTLKQIAKEILVNEEDIKGYRVTS | |
| | | TGKPEFTNLKVYHDIKDITARKEIIENAELLDQIAKILTIYQ | |
| | | SSEDIQEELTNLNSELTQEEIEQISNLKGYTGTHNLSLKAIN | |
| | | LILDELWHTNDNQIAIFNRLKLVPKKVDLSQQKEIPTTLVDD | |
| | | FILSPVVKRSFIQSIKVINAIIKKYGLPNDIIIELAREKNSK | |
| | | DAQKMINEMQKRNRQTNERIEEIIRTTGKENAKYLIEKIKLH | |
| | | DMQEGKCLYSLEAIPLEDLLNNPFNYEVDHIIPRSVSEDNSF | |
| | | NNKVLVKQEENSKKGNRTPFQYLSSSDSKISYETFKKHILNL | |
| | | AKGKGRISKTKKEYLLEERDINRFSVQKDFINRNLVDTRYAT | |
| | | RGLMNLLRSYFRVNNLDVKVKSINGGFTSFLRRKWKFKKERN | |
| | | KGYKHHAEDALIIANADFIFKEWKKLDKAKKVMENQMFEEKQ | |
| | | AESMPEIETEQEYKEIFITPHQIKHIKDFKDYKYSHRVDKKP | |
| | | NRELINDTLYSTRKDDKGNTLIVNNLNGLYDKDNDKLKKLIN | |
| | | KSPEKLLMYHHDPQTYQKLKLIMEQYGDEKNPLYKYYEETGN | |
| | | YLTKYSKKDNGPVIKKIKYYGNKLNAHLDITDDYPNSRNKVV | |
| | | KLSLKPYRFDVYLDNGVYKFVTVKNLDVIKKENYYEVNSKCY | |
| | | EEAKKLKKISNQAEFIASFYNNDLIKINGELYRVIGVNNDLL | |
| | | NRIEVNMIDITYREYLENMNDKRPPRIIKTIASKTQSIKKYS | |
| | | TDILGNLYEVKSKKHPQIIKKG | |
| pBE0123 | 45 | YWMRHALTLAKRARDEREVPVGAVLVLNNRVIGEGWNRAIGL | 75-90% |
| | | HDPTAHAEIMALRQGGLVMQNYRLIDATLYVTFEPCVMCAGA | (A5) |
| | | MIHSRIGRVVFGVRNSKRGAAGSLMNVLNYPGMNHRVEITEG | 50-75% |
| | | ILADECAALLCDFYRMPRQVFNAQKKPAPAPILGLAIGITSV | (A8) |
| | | GYGIIDYETRDVIDAGVRLFKEANVENNEGRRSKRGARRLKR | |
| | | RRRHRIQRVKKLLFDYNLLTDHSELSGINPYEARVKGLSQKL | |
| | | SEEEFSAALLHLAKRRGVHNVNEVEEDTGNELSTKEQISRNS | |
| | | KALEEKYVAELQLERLKKDGEVRGSINRFKTSDYVKEAKQLL | |
| | | KVQKAYHQLDQSFIDTYIDLLETRRTYYEGPGEGSPFGWKDI | |
| | | KEWYEMLMGHCTYFPEELRSVKYAYNADLYNALNDLNNLVIT | |
| | | RDENEKLEYYEKFQIIENVFKQKKKPTLKQIAKEILVNEEDI | |
| | | KGYRVTSTGKPEFTNLKVYHDIKDITARKEIIENAELLDQIA | |
| | | KILTIYQSSEDIQEELTNLNSELTQEEIEQISNLKGYTGTHN | |
| | | LSLKAINLILDELWHTNDNQIAIFNRLKLVPKKVDLSQQKEI | |
| | | PTTLVDDFILSPVVKRSFIQSIKVINAIIKKYGLPNDIIIEL | |
| | | AREKNSKDAQKMINEMQKRNRQTNERIEEIIRTTGKENAKYL | |
| | | IEKIKLHDMQEGKCLYSLEAIPLEDLLNNPFNYEVDHIIPRS | |
| | | VSFDNSFNNKVLVKQEENSKKGNRTPFQYLSSSDSKISYETF | |
| | | KKHILNLAKGKGRISKTKKEYLLEERDINRFSVQKDFINRNL | |
| | | VDTRYATRGLMNLLRSYFRVNNLDVKVKSINGGFTSELRRKW | |
| | | KFKKERNKGYKHHAEDALIIANADFIFKEWMFEEKQAESMPE | |
| | | IETEQEYKEIFITPHQIKHIKDFKDYKYSHRVDKKPNRELIN | |
| | | DTLYSTRKDDKGNTLIVNNLNGLYDKDNDKLKKLINKSPEKL | |
| | | LMYHHDPQTYQKLKLIMEQYGDEKNPLYKYYEETGNYLTKYS | |
| | | KKDNGPVIKKIKYYGNKLNAHLDITDDYPNSRNKVVKLSLKP | |
| | | YRFDVYLDNGVYKFVTVKNLDVIKKENYYEVNSKCYEEAKKL | |
| | | KKISNQAEFIASFYNNDLIKINGELYRVIGVNNDLLNRIEVN | |
| | | MIDITYREYLENMNDKRPPRIIKTIASKTQSIKKYSTDILGN | |
| | | LYEVKSKKHPQIIKKG | |
| pBE0124 | 46 | YWMRHALTLAKRARDEREVPVGAVLVLNNRVIGEGWNRAIGL | 30-50% |
| | | HDPTAHAEIMALRQGGLVMQNYRLIDATLYVTFEPCVMCAGA | (A5 and |
| | | MIHSRIGRVVFGVRNSKRGAAGSLMNVLNYPGMNHRVEITEG | A8) |
| | | ILADECAALLCDFYRMPRQVFNAQKKPAPAPILGLAIGITSV | |
| | | GYGIIDYETRDVIDAGVRLFKEANVENNEGRRSKRGARRLKR | |
| | | RRRHRIQRVKKLLFDYNLLTDHSELSGINPYEARVKGLSQKL | |
| | | SEEEFSAALLHLAKRRGVHNVNEVEEDTGNELSTKEQISRNS | |
| | | KALEEKYVAELQLERLKKDGEVRGSINRFKTSDYVKEAKQLL | |
| | | KVQKAYHQLDQSFIDTYIDLLETRRTYYEGPGEGSPFGWKDI | |
| | | KEWYEMLMGHCTYFPEELRSVKYAYNADLYNALNDLNNLVIT | |
| | | RDENEKLEYYEKFQIIENVFKQKKKPTLKQIAKEILVNEEDI | |
| | | KGYRVTSTGKPEFTNLKVYHDIKDITARKEIIENAELLDQIA | |
| | | KILTIYQSSEDIQEELTNLNSELTQEEIEQISNLKGYTGTHN | |
| | | LSLKAINLILDELWHTNDNQIAIFNRLKLVPKKVDLSQQKEI | |
| | | PTTLVDDFILSPVVKRSFIQSIKVINAIIKKYGLPNDIIIEL | |
| | | AREKNSKDAQKMINEMQKRNRQTNERIEEIIRTTGKENAKYL | |
| | | IEKIKLHDMQEGKCLYSLEAIPLEDLLNNPFNYEVDHIIPRS | |
| | | VSFDNSFNNKVLVKQEENSKKGNRTPFQYLSSSDSKISYETF | |
| | | KKHILNLAKGKGRISKTKKEYLLEERDINRFSVQKDFINRNL | |
| | | VDTRYATRGLMNLLRSYFRVNNLDVKVKSINGGFTSFLRRKF | |
| | | KKERNKGYKHHAEDALIIANADFIFKDKAKKVMENQMFEEKQ | |
| | | AESMPEIETEQEYKEIFITPHQIKHIKDFKDYKYSHRVDKKP | |
| | | NRELINDTLYSTRKDDKGNTLIVNNLNGLYDKDNDKLKKLIN | |
| | | KSPEKLLMYHHDPQTYQKLKLIMEQYGDEKNPLYKYYEETGN | |
| | | YLTKYSKKDNGPVIKKIKYYGNKLNAHLDITDDYPNSRNKVV | |
| | | KLSLKPYRFDVYLDNGVYKFVTVKNLDVIKKENYYEVNSKCY | |
| | | EEAKKLKKISNQAEFIASFYNNDLIKINGELYRVIGVNNDLL | |
| | | NRIEVNMIDITYREYLENMNDKRPPRIIKTIASKTQSIKKYS | |
| | | TDILGNLYEVKSKKHPQIIKKG | |
| pBE0125 | 47 | YWMRHALTLAKRARDEREVPVGAVLVLNNRVIGEGWNRAIGL | >90% |
| | | HDPTAHAEIMALRQGGLVMQNYRLIDATLYVTFEPCVMCAGA | (A5) |
| | | MIHSRIGRVVFGVRNSKRGAAGSLMNVLNYPGMNHRVEITEG | 75-90% |
| | | ILADECAALLCDFYRMPRQVFNAQKKPAPILGLAIGITSVGY | (A8) |
| | | GIIDYETRDVIDAGVRLFKEANVENNEGRRSKRGARRLKRRR | |
| | | RHRIQRVKKLLFDYNLLTDHSELSGINPYEARVKGLSQKLSE | |
| | | EEFSAALLHLAKRRGVHNVNEVEEDTGNELSTKEQISRNSKA | |
| | | LEEKYVAELQLERLKKDGEVRGSINRFKTSDYVKEAKQLLKV | |
| | | QKAYHQLDQSFIDTYIDLLETRRTYYEGPGEGSPFGWKDIKE | |
| | | WYEMLMGHCTYFPEELRSVKYAYNADLYNALNDLNNLVITRD | |
| | | ENEKLEYYEKFQIIENVFKQKKKPTLKQIAKEILVNEEDIKG | |
| | | YRVTSTGKPEFTNLKVYHDIKDITARKEIIENAELLDQIAKI | |
| | | LTIYQSSEDIQEELTNLNSELTQEEIEQISNLKGYTGTHNLS | |
| | | LKAINLILDELWHTNDNQIAIFNRLKLVPKKVDLSQQKEIPT | |
| | | TLVDDFILSPVVKRSFIQSIKVINAIIKKYGLPNDIIIELAR | |
| | | EKNSKDAQKMINEMQKRNRQTNERIEEIIRTTGKENAKYLIE | |
| | | KIKLHDMQEGKCLYSLEAIPLEDLLNNPFNYEVDHIIPRSVS | |
| | | FDNSFNNKVLVKQEENSKKGNRTPFQYLSSSDSKISYETFKK | |
| | | HILNLAKGKGRISKTKKEYLLEERDINRFSVQKDFINRNLVD | |
| | | TRYATRGLMNLLRSYFRVNNLDVKVKSINGGFTSFLRRKWKF | |
| | | KKERNKGYKHHAEDALIIANADFIFKEWKKLDKAKKVMENQM | |
| | | FEEKQAESMPEIETEQEYKEIFITPHQIKHIKDFKDYKYSHR | |
| | | VDKKPNRELINDTLYSTRKDDKGNTLIVNNLNGLYDKDNDKL | |
| | | KKLINKSPEKLLMYHHDPQTYQKLKLIMEQYGDEKNPLYKYY | |
| | | EETGNYLTKYSKKDNGPVIKKIKYYGNKLNAHLDITDDYPNS | |
| | | RNKVVKLSLKPYRFDVYLDNGVYKFVTVKNLDVIKKENYYEV | |
| | | NSKCYEEAKKLKKISNQAEFIASFYNNDLIKINGELYRVIGV | |
| | | NNDLLNRIEVNMIDITYREYLENMNDKRPPRIIKTIASKTQS | |
| | | IKKYSTDILGNLYEVKSKKHPQIIKKG | |
| pBE0126 | 48 | YWMRHALTLAKRARDEREVPVGAVLVLNNRVIGEGWNRAIGL | 75-90% |
| | | HDPTAHAEIMALRQGGLVMQNYRLIDATLYVTFEPCVMCAGA | (A5) |
| | | MIHSRIGRVVFGVRNSKRGAAGSLMNVLNYPGMNHRVEITEG | |
| | | ILADECAALLCDFYRMPRQVFNAQKKPAPAPILGLAIGITSV | |
| | | GYGIIDYETRDVIDAGVRLFKEANVENNEGRRSKRGARRLKR | |
| | | RRRHRIQRVKKLLFDYNLLTDHSELSGINPYEARVKGLSQKL | |
| | | SEEEFSAALLHLAKRRGVHNVNEVEKYVAELQLERLKKDGEV | |
| | | RGSINRFKTSDYVKEAKQLLKVQKAYHQLDQSFIDTYIDLLE | |
| | | TRRTYYEGPGEGSPFGWKDIKEWYEMLMGHCTYFPEELRSVK | |
| | | YAYNADLYNALNDLNNLVITRDENEKLEYYEKFQIIENVFKQ | |
| | | KKKPTLKQIAKEILVNEEDIKGYRVTSTGKPEFTNLKVYHDI | |
| | | KDITARKEIIENAELLDQIAKILTIYQSSEDIQEELTNLNSE | |
| | | LTQEEIEQISNLKGYTGTHNLSLKAINLILDELWHTNDNQIA | |
| | | IFNRLKLVPKKVDLSQQKEIPTTLVDDFILSPVVKRSFIQSI | |
| | | KVINAIIKKYGLPNDIIIELAREKNSKDAQKMINEMQKRNRQ | |
| | | TNERIEEIIRTTGKENAKYLIEKIKLHDMQEGKCLYSLEAIP | |
| | | LEDLLNNPFNYEVDHIIPRSVSFDNSFNNKVLVKQEENSKKG | |
| | | NRTPFQYLSSSDSKISYETFKKHILNLAKGKGRISKTKKEYL | |
| | | LEERDINRFSVQKDFINRNLVDTRYATRGLMNLLRSYFRVNN | |
| | | LDVKVKSINGGFTSFLRRKWKFKKERNKGYKHHAEDALIIAN | |
| | | ADFIFKEWKKLDKAKKVMENQMFEEKQAESMPEIETEQEYKE | |
| | | IFITPHQIKHIKDFKDYKYSHRVDKKPNRELINDTLYSTRKD | |
| | | DKGNTLIVNNLNGLYDKDNDKLKKLINKSPEKLLMYHHDPQT | |
| | | YQKLKLIMEQYGDEKNPLYKYYEETGNYLTKYSKKDNGPVIK | |
| | | KIKYYGNKLNAHLDITDDYPNSRNKVVKLSLKPYRFDVYLDN | |
| | | GVYKFVTVKNLDVIKKENYYEVNSKCYEEAKKLKKISNQAEF | |
| | | IASFYNNDLIKINGELYRVIGVNNDLLNRIEVNMIDITYREY | |
| | | LENMNDKRPPRIIKTIASKTQSIKKYSTDILGNLYEVKSKKH | |
| | | PQIIKKG | |
| pBE0127 | 49 | YWMRHALTLAKRARDEREVPVGAVLVLNNRVIGEGWNRAIGL | 30-50% |
| | | HDPTAHAEIMALRQGGLVMQNYRLIDATLYVTFEPCVMCAGA | (A5) |
| | | MIHSRIGRVVFGVRNSKRGAAGSLMNVLNYPGMNHRVEITEG | |
| | | ILADECAALLCDFYRMPRQVFNAQKKPAPAPILGLAIGITSV | |
| | | GYGIIDYETRDVIDAGVRLFKEANVENNEGRRSKRGARRLKR | |
| | | RRRHRIQRVKKLLFDYNLLTDHSELSGINPYEARVKGLSQKL | |
| | | SEEEFSAALLHLAKRRGVHNVNEVEKYVAELQLERLKKDGEV | |
| | | RGSINRFKTSDYVKEAKQLLKVQKAYHQLDQSFIDTYIDLLE | |
| | | TRRTYYEGPGEGSPFGWKDIKEWYEMLMGHCTYFPEELRSVK | |
| | | YAYNADLYNALNDLNNLVITRDENEKLEYYEKFQIIENVFKQ | |
| | | KKKPTLKQIAKEILVNEEDIKGYRVTSTGKPEFTNLKVYHDI | |
| | | KDITARKEIIENAELLDQIAKILTIYQSSEDIQEELTNLNSE | |
| | | LTQEEIEQISNLKGYTGTHNLSLKAINLILDELWHTNDNQIA | |
| | | IFNRLKLVPKKVDLSQQKEIPTTLVDDFILSPVVKRSFIQSI | |
| | | KVINAIIKKYGLPNDIIIELAREKNSKDAQKMINEMQKRNRQ | |
| | | TNERIEEIIRTTGKENAKYLIEKIKLHDMQEGKCLYSLEAIP | |
| | | LEDLLNNPFNYEVDHIIPRSVSEDNSFNNKVLVKQEENSKKG | |
| | | NRTPFQYLSSSDSKISYETFKKHILNLAKGKGRISKTKKEYL | |
| | | LEERDINRFSVQKDFINRNLVDTRYATRGLMNLLRSYFRVNN | |
| | | LDVKVKSINGGFTSFLRRKWKFKKERNKGYKHHAEDALIIAN | |
| | | ADFIFKEWMFEEKQAESMPEIETEQEYKEIFITPHQIKHIKD | |
| | | FKDYKYSHRVDKKPNRELINDTLYSTRKDDKGNTLIVNNLNG | |
| | | LYDKDNDKLKKLINKSPEKLLMYHHDPQTYQKLKLIMEQYGD | |
| | | EKNPLYKYYEETGNYLTKYSKKDNGPVIKKIKYYGNKLNAHL | |
| | | DITDDYPNSRNKVVKLSLKPYRFDVYLDNGVYKFVTVKNLDV | |
| | | IKKENYYEVNSKCYEEAKKLKKISNQAEFIASFYNNDLIKIN | |
| | | GELYRVIGVNNDLLNRIEVNMIDITYREYLENMNDKRPPRII | |
| | | KTIASKTQSIKKYSTDILGNLYEVKSKKHPQIIKKG | |
| pBE0129 | 64 | YWMRHALTLAKRARDEREVPVGAVLVLNNRVIGEGWNRAIGL | 0% |
| | | HDPTAHAEIMALRQGGLVMQNYRLIDATLYVTFEPCVMCAGA | |
| | | MIHSRIGRVVFGVRNSKRGAAGSLMNVLNYPGMNHRVEITEG | |
| | | ILADECAALLCDFYRMPRQVFNAQKKPAPILGLAIGITSVGY | |
| | | GIIDYETRDVIDAGVRLFKEANVENNEGRRSKRGARRLKRRR | |
| | | RHRIQRVKKLLFDYNLLTDHSELSGINPYEARVKGLSQKLSE | |
| | | EEFSAALLHLAKRRGVHNVNEVEKYVAELQLERLKKDGEVRG | |
| | | SINRFKTSDYVKEAKQLLKVQKAYHQLDQSFIDTYIDLLETR | |
| | | RTYYEGPGEGSPFGWKDIKEWYEMLMGHCTYFPEELRSVKYA | |
| | | YNADLYNALNDLNNLVITRDENEKLEYYEKFQIIENVFKQKK | |
| | | KPTLKQIAKEILVNEEDIKGYRVTSTGKPEFTNLKVYHDIKD | |
| | | ITARKEIIENAELLDQIAKILTIYQSSEDIQEELTNLNSELT | |
| | | QEEIEQISNLKGYTGTHNLSLKAINLILDELWHTNDNQIAIF | |
| | | NRLKLVPKKVDLSQQKEIPTTLVDDFILSPVVKRSFIQSIKV | |
| | | INAIIKKYGLPNDIIIELAREKNSKDAQKMINEMQKRNRQTN | |
| | | ERIEEIIRTTGKENAKYLIEKIKLHDMQEGKCLYSLEAIPLE | |
| | | DLLNNPFNYEVDHIIPRSVSFDNSFNNKVLVKQEENSKKGNR | |
| | | TPFQYLSSSDSKISYETFKKHILNLAKGKGRISKTKKEYLLE | |
| | | ERDINRFSVQKDFINRNLVDTRYATRGLMNLLRSYFRVNNLD | |
| | | VKVKSINGGFTSFLRRKFKKERNKGYKHHAEDALIIANADFI | |
| | | FKDKAKKVMENQMFEEKQAESMPEIETEQEYKEIFITPHQIK | |
| | | HIKDFKDYKYSHRVDKKPNRELINDTLYSTRKDDKGNTLIVN | |
| | | NLNGLYDKDNDKLKKLINKSPEKLLMYHHDPQTYQKLKLIME | |
| | | QYGDEKNPLYKYYEETGNYLTKYSKKDNGPVIKKIKYYGNKL | |
| | | NAHLDITDDYPNSRNKVVKLSLKPYRFDVYLDNGVYKFVTVK | |
| | | NLDVIKKENYYEVNSKCYEEAKKLKKISNQAEFIASFYNNDL | |
| | | IKINGELYRVIGVNNDLLNRIEVNMIDITYREYLENMNDKRP | |
| | | PRIIKTIASKTQSIKKYSTDILGNLYEVKSKKHPQIIKKG | |

In a separate experiment, nucleobase editing activity of modified BEs comprising the amino acid sequence of SEQ ID NO: 76 or 77 was tested. Each modified BE was found to edit 9% of adenines at position 13 of SEQ ID NO: 4 to guanines. The sequences for SEQ ID Nos: 76 and 77 are presented below where the TadA is presented as bolded text, the linker between the deaminase and the nuclease as italicized, and the nuclease as regular font.

SEQ ID NO: 76 (ABE8e_SaCas9_delta_E506-K623):
MSEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRIG
RVVFGVRNSKRGAAGSLMNVLNYPGMNHRVEITEGILADECAALLCDFYR
MPRQVFNAQKKAQSSINSGGSSGGSSGSETPGTSESATPESSGGSSGGSK
RNYILGLAIGITSVGYGIIDYETRDVIDAGVRLFKEANVENNEGRRSKRG
ARRLKRRRRHRIQRVKKLLFDYNLLTDHSELSGINPYEARVKGLSQKLSE
EEFSAALLHLAKRRGVHNVNEVEEDTGNELSTKEQISRNSKALEEKYVAE
LQLERLKKDGEVRGSINRFKTSDYVKEAKQLLKVQKAYHQLDQSFIDTYI
DLLETRRTYYEGPGEGSPFGWKDIKEWYEMLMGHCTYFPEELRSVKYAYN
ADLYNALNDLNNLVITRDENEKLEYYEKFQIIENVFKQKKKPTLKQIAKE
ILVNEEDIKGYRVTSTGKPEFTNLKVYHDIKDITARKEIIENAELLDQIA
KILTIYQSSEDIQEELTNLNSELTQEEIEQISNLKGYTGTHNLSLKAINL
ILDELWHTNDNQIAIFNRLKLVPKKVDLSQQKEIPTTLVDDFILSPVVKR
SFIQSIKVINAIIKKYGLPNDIIIELAREKNSKDAQKMINEMQKRNRQTN
ERIEYLLEERDINRFSVQKDFINRNLVDTRYATRGLMNLLRSYFRVNNLD
VKVKSINGGFTSFLRRKWKFKKERNKGYKHHAEDALIIANADFIFKEWKK
LDKAKKVMENQMFEEKQAESMPEIETEQEYKEIFITPHQIKHIKDFKDYK
YSHRVDKKPNRELINDTLYSTRKDDKGNTLIVNNLNGLYDKDNDKLKKLI
NKSPEKLLMYHHDPQTYQKLKLIMEQYGDEKNPLYKYYEETGNYLTKYSK
KDNGPVIKKIKYYGNKLNAHLDITDDYPNSRNKVVKLSLKPYRFDVYLDN
GVYKFVTVKNLDVIKKENYYEVNSKCYEEAKKLKKISNQAEFIASFYNND
LIKINGELYRVIGVNNDLLNRIEVNMIDITYREYLENMNDKRPPRIIKTI
ASKTQSIKKYSTDILGNLYEVKSKKHPQIIKKG
SEQ ID NO: 77 (ABE8e_SaCas9_delta_K2-Y5_K719-Q731_
E506-K623):
MSEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRIG
RVVFGVRNSKRGAAGSLMNVLNYPGMNHRVEITEGILADECAALLCDFYR
MPRQVFNAQKKAQSSINSGGSSGGSSGSETPGTSESATPESSGGSSGGSI
LGLAIGITSVGYGIIDYETRDVIDAGVRLFKEANVENNEGRRSKRGARRL
KRRRRHRIQRVKKLLFDYNLLTDHSELSGINPYEARVKGLSQKLSEEEFS
AALLHLAKRRGVHNVNEVEEDTGNELSTKEQISRNSKALEEKYVAELQLE
RLKKDGEVRGSINRFKTSDYVKEAKQLLKVQKAYHQLDQSFIDTYIDLLE
TRRTYYEGPGEGSPFGWKDIKEWYEMLMGHCTYFPEELRSVKYAYNADLY
NALNDLNNLVITRDENEKLEYYEKFQIIENVFKQKKKPTLKQIAKEILVN
EEDIKGYRVTSTGKPEFTNLKVYHDIKDITARKEIIENAELLDQIAKILT
IYQSSEDIQEELTNLNSELTQEEIEQISNLKGYTGTHNLSLKAINLILDE
LWHTNDNQIAIFNRLKLVPKKVDLSQQKEIPTTLVDDFILSPVVKRSFIQ
SIKVINAIIKKYGLPNDIIIELAREKNSKDAQKMINEMQKRNRQTNERIE
YLLEERDINRFSVQKDFINRNLVDTRYATRGLMNLLRSYFRVNNLDVKVK
SINGGFTSFLRRKWKFKKERNKGYKHHAEDALIIANADFIFKEWMFEEKQ
AESMPEIETEQEYKEIFITPHQIKHIKDFKDYKYSHRVDKKPNRELINDT
LYSTRKDDKGNTLIVNNLNGLYDKDNDKLKKLINKSPEKLLMYHHDPQTY
QKLKLIMEQYGDEKNPLYKYYEETGNYLTKYSKKDNGPVIKKIKYYGNKL
NAHLDITDDYPNSRNKVVKLSLKPYRFDVYLDNGVYKFVTVKNLDVIKKE
NYYEVNSKCYEEAKKLKKISNQAEFIASFYNNDLIKINGELYRVIGVNND
LLNRIEVNMIDITYREYLENMNDKRPPRIIKTIASKTQSIKKYSTDILGN
LYEVKSKKHPQIIKKG

### Description of Sequences

Table 2 is a description of the sequences included in the electronic sequence listing. The table provides the SEQ ID NO, a description of the sequence, and the length of the sequence in amino acids. These sequences are included in the aforementioned electronic sequence listing (41816_PCT_SequenceListing; Size: 172 KB; and Date of Creation: May 6, 2024) incorporated by reference in its entirety.

TABLE 2

| SEQ | | |
|---|---|---|
| ID NO: | Description | Size |
| 1 | TadA | 166 |
| 2 | nSaCas9 | 1052 |
| 3 | Reference nucleobase editor | 1250 |
| 4 | Activity Assessment Polynucleotide | 22 |
| 5 | c-myc NLS | 9 |
| 6 | NLS Linker | 4 |
| 7 | SV40 NLS | 7 |
| 8 | NLS linker | 15 |
| 9 | nucleoplasmin NLS | 16 |
| 10 | NLS Linker | 4 |
| 11 | linker | 3 |
| 12 | linker | 5 |
| 13 | linker | 7 |
| 14 | pBE0015 | 1223 |
| 15 | pBE0016 | 1225 |
| 16 | pBE0119 | 1221 |
| 17 | pBE0019 | 1246 |
| 18 | pBE0020 | 1246 |
| 19 | pBE0021 | 1242 |
| 20 | pBE0057 | 1244 |
| 21 | pBE0058 | 1242 |
| 22 | pBE0059 | 1244 |
| 23 | pBE0022 | 1247 |
| 24 | pBE0024 | 1244 |
| 25 | pBE0025 | 1246 |
| 26 | pBE0026 | 1246 |
| 27 | pBE0029 | 1237 |
| 28 | pBE0038 | 1228 |
| 29 | pBE0040 | 1241 |
| 30 | pBE0042 | 1236 |
| 31 | pBE0043 | 1246 |
| 32 | pBE0060 | 1215 |
| 33 | pBE0061 | 1208 |
| 34 | pBE0105 | 1207 |
| 35 | pBE0107 | 1207 |
| 36 | pBE0108 | 1209 |
| 37 | pBE0109 | 1205 |
| 38 | pBE0110 | 1207 |
| 39 | pBE0116 | 1238 |
| 40 | pBE0117 | 1240 |
| 41 | pBE0118 | 1238 |
| 42 | pBE0120 | 1244 |
| 43 | pBE0121 | 1242 |
| 44 | pBE0122 | 1240 |
| 45 | pBE0123 | 1192 |
| 46 | pBE0124 | 1198 |
| 47 | pBE0125 | 1203 |
| 48 | pBE0126 | 1183 |
| 49 | pBE0127 | 1170 |
| 50 | Del3 | 1242 |
| 51 | Del4 | 1069 |
| 52 | Del5 | 1241 |
| 53 | Del6 | 1247 |
| 54 | Del7 | 1248 |
| 55 | Del8 | 1246 |
| 56 | Del9 | 1243 |
| 57 | pBE0027 | 1248 |
| 58 | pBE0028 | 1246 |
| 59 | Del2 | 1194 |
| 60 | pBE0031 | 1248 |
| 61 | pBE0039 | 1236 |
| 62 | pBE0041 | 1243 |
| 63 | pBE0115 | 1240 |
| 64 | pBE0129 | 1174 |
| 65 | SluCas9 protein | 1053 |
| 66 | sRGN1 | 1057 |
| 67 | sRGN2 | 1053 |
| 68 | sRGN3 | 1057 |
| 69 | sRGN3.1 | 1057 |
| 70 | sRGN3.2 | 1057 |
| 71 | sRGN3.3 | 1057 |
| 72 | sRGN4 | 1054 |
| 73 | KRAB Domain | 71 |
| 74 | Dnmt3A | 301 |
| 75 | Dnmt3L | 215 |
| 76 | ABE8e_SaCas9_delta_E506-K623 | 1133 |
| 77 | ABE8e_SaCas9_delta_K2-Y5_K719-Q731_E506- | 1116 |
| | K623 | |
| 78 | Linker | 32 |
| 79 | Linker | 4 |
| 80 | Linker | 6 |
| 81 | Linker | 8 |

What is claimed is:

1. A nucleobase editor comprising a nucleotide deaminase, a Cas9 nuclease, and a linker between the deaminase and Cas9 nuclease, wherein the nucleobase editor is between 1175-1249 amino acids in length.

2. The nucleobase editor of claim 1, wherein the nucleobase editor is between 1175-1249, 1180-1249, 1190-1249, 1200-1249, 1210-1249, 1220-1249, 1230-1249, 1240-1249, 1180-1200, 1180-1190, 1200-1249, 1200-1240, 1200-1230, 1200-1220, 1200-1210, 1210-1249, 1210-1240, 1210-1230, 1210-1220, 1220-1249, 1220-1240, 1220-1230, 1230-1249, 1230-1240, or 1240-1249 amino acids in length.

3. The nucleobase editor of any one of claims 1 or 2, wherein the linker between the deaminase and Cas9 nuclease is 2-10, 3-7, 3-5, or 3 amino acids in length.

4. The nucleobase editor of any one of claims 1-3, wherein the nucleotide deaminase is 145-166, 145-162, 145-158, 145-154, 145-151, 150-166, 150-162, 150-156, 150-153, 152-166, 152-158, 152-154, 157-166, 157-162, 161-166, or 161-163 amino acids in length.

5. The nucleobase editor of any one of claims 1-4, wherein the Cas9 nuclease is between 1010-1052, 1010-1040, 1010-1025, 1010-1015, 1020-1052, 1020-1040, 1020-1030, 1030-1052, 1030-1040, 1030-1035, 1035-1046, 1040-1052, 1040-1046, 1045-1052, or 1045-1048 amino acids in length.

6. The nucleobase editor of any one of claims 1-5, wherein the linker between the deaminase and Cas9 nuclease comprises one or more proline and one or more alanine.

7. The nucleobase editor of any one of claims 1-6, wherein the nucleotide deaminase comprises an amino acid sequence that is at least 80%, 85%, 90%, 91%, 92%, 93%, 94%, 95%, 96%, 97%, 98%, 99% or 100% identical to the amino acid sequence of SEQ ID NO: 1, or a functional fragment of SEQ ID NO: 1.

8. The nucleobase editor of claim 7, wherein the nucleotide deaminase lacks the amino acids corresponding to the first 1, 2, 3, 4, 5, 6, 7, or 8 amino acids of SEQ ID NO: 1.

9. The nucleobase editor of claim 7 or 8, wherein the nucleotide deaminase lacks the amino acids corresponding to the last 1, 2, 3, 4, 5, 6, 7, 8, 9, or 10 amino acids of SEQ ID NO: 1.

10. The nucleobase editor of claim 7 or 8, wherein the nucleotide deaminase lacks the amino acids corresponding to the first 6 amino acids of SEQ ID NO: 1 and lacks the amino acids corresponding to the last 8 amino acids of SEQ ID NO: 1.

11. The nucleobase editor of any one of claims 1-10, wherein the Cas9 nuclease comprises an amino acid sequence that is at least 80%, 85%, 90%, 91%, 92%, 93%, 94%, 95%, 96%, 97%, 98%, 99% or 100% identical to the amino acid sequence of SEQ ID NO: 2, or a functional fragment SEQ ID NO: 2.

12. The nucleobase editor of any one of claims 1-12, wherein the Cas9 nuclease lacks one or more amino acids from the range of amino acids corresponding to amino acids 1-4, 1-6, 77-85, 124-126, 124-129, 124-143, 124-145, 131-134, 135-138, 122-129, 505-622, 687-688, 716-720, 718-719, 718-730, 765, and/or 865-873 of SEQ ID NO: 2.

13. The nucleobase editor of any one of claims 1-12, wherein the Cas9 nuclease lacks the amino acids corresponding to amino acids 1-4, 1-6, 77-85, 124-126, 124-129, 124-143, 124-145, 131-134, 135-138, 122-129, 505-622, 687-688, 716-720, 718-719, 718-730, 765, and/or 865-873 of SEQ ID NO: 2.

14. The nucleobase editor of any one of claims 1-13, wherein the Cas9 nuclease lacks the amino acids corresponding to amino acids:
a) 718-719 and 765 of SEQ ID NO: 2,
b) 718-719 and 687-688 of SEQ ID NO: 2,
c) 716-720 and 687-688 of SEQ ID NO: 2,
d) 1-4 and 718-730 of SEQ ID NO: 2,
e) 1-4, 687-688, and 716-720 of SEQ ID NO: 2,
f) 1-4 and 124-145 of SEQ ID NO: 2, or
g) 1-4, 505-622, and 718-730.

15. The nucleobase editor of any one of claims 1-14, wherein the Cas9 nuclease does not lack one or more amino acids from the range of amino acids corresponding to amino acids:
a) 1-8 of SEQ ID NO: 2,
b) 1-10 of SEQ ID NO: 2,
c) 81-94 of SEQ ID NO: 2,
d) 163-164 of SEQ ID NO: 2,
e) 181-184 of SEQ ID NO: 2,
f) 244-424 of SEQ ID NO: 2,
g) 714-769 of SEQ ID NO: 2,
h) 736-742 of SEQ ID NO: 2,
i) 978-979 of SEQ ID NO: 2, and/or
j) 1039-1052 of SEQ ID NO: 2.

16. The nucleobase editor of any one of claims 1-15, wherein the Cas9 nuclease does not lack the amino acid ranges corresponding to amino acids:
a) 1-8 of SEQ ID NO: 2,
b) 1-10 of SEQ ID NO: 2,
c) 81-94 of SEQ ID NO: 2,
d) 163-164 of SEQ ID NO: 2,
e) 181-184 of SEQ ID NO: 2,
f) 244-424 of SEQ ID NO: 2,
g) 714-769 of SEQ ID NO: 2,
h) 736-742 of SEQ ID NO: 2,
i) 978-979 of SEQ ID NO: 2, and/or
j) 1039-1052 of SEQ ID NO: 2.

17. The nucleobase editor of any one of claims 1-16, wherein the nucleobase editor has at least 50%, 60%, 70%, 80%, or 90% base editing activity as compared to a nucleobase editor comprising the sequence of SEQ ID NO: 3.

18. The nucleobase editor of claim 17, wherein the base editing activity is assessed using a polynucleotide comprising the nucleotide sequence of SEQ ID NO: 4.

19. The nucleobase editor of claim 18, wherein the base editing activity is assessed at position A5, A8 or A13 of SEQ ID NO: 4.

20. The nucleobase editor of any one of claims 1-19, wherein the nucleobase editor further comprises one or more nuclear localization signals (NLSs) and optionally one or more linkers fusing the one or more NLSs to the nucleobase editor.

21. The nucleobase editor of claim 20, wherein the one or more NLSs are selected from a c-myc NLS (e.g., an NLS comprising the amino acid sequence of SEQ ID NO: 5), an SV40 NLS (e.g., an NLS comprising the amino acid sequence of SEQ ID NO: 7), and/or a nucleoplasmin NLS (e.g., an NLS comprising the amino acid sequence of SEQ ID NO: 9).

22. The nucleobase editor of claim 20 or 21, wherein the nucleobase editor comprises: a) a first NLS conjugated to the N-terminus of the nucleobase editor, optionally by means of a first NLS linker, and b) a second NLS conjugated to the C-terminus of the nucleobase editor, optionally by means of a second NLS linker.

23. The nucleobase editor of claim 22, wherein the nucleobase editor further comprises: c) a third NLS conjugated to the second NLS, optionally by means of a third NLS linker.

24. The nucleobase editor of claim 23, wherein the first NLS comprises the amino acid sequence of SEQ ID NO: 5, wherein the optional first NLS linker comprises the amino acid sequence of SEQ ID NO: 6, wherein the second NLS comprises the amino acid sequence of SEQ ID NO: 7, wherein the optional second NLS linker comprises the amino acid sequence of SEQ ID NO: 8, wherein the third NLS comprises the amino acid sequence of SEQ ID NO: 9, and wherein the optional third NLS linker comprises the amino acid sequence of SEQ ID NO: 10.

25. The nucleobase editor of any one of claims 20-24, wherein the one or more NLSs and one or more linkers fusing the one or more NLSs to the nucleobase editor are collectively no more than 40, 37, 35, 32, 28, or 26 amino acids.

26. The nucleobase editor of claim 25, wherein the one or more NLSs and one or more linkers fusing the one or more NLSs to the nucleobase editor are collectively no more than 32 amino acids.

27. The nucleobase editor of any one of the previous claims, wherein the amino acid sequence of the linker is selected from the group of SEQ ID NO: 11, SEQ ID NO: 12, and SEQ ID NO: 13.

28. The nucleobase editor of any one of the previous claims, wherein the amino acid sequence of the linker between the deaminase and Cas9 nuclease is SEQ ID NO: 11.

29. The nucleobase editor of any one of claims 1-27, wherein the amino acid sequence of the linker between the deaminase and Cas9 nuclease is SEQ ID NO: 12.

30. The nucleobase editor of any one of claims 1-27, wherein the amino acid sequence of the linker between the deaminase and Cas9 nuclease is SEQ ID NO: 13.

31. The nucleobase editor of any one of the previous claims, wherein the amino acid sequence which encodes the nucleobase editor is at least 80%, 85%, 90%, 91%, 92%, 93%, 94%, 95%, 96%, 97%, 98%, 99% or 100% identical to SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, or SEQ ID NO: 56

32. The nucleobase editor of any one of the previous claims, wherein the amino acid sequence which encodes the nucleobase editor is selected from the group SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, and SEQ ID NO: 56.

33. The nucleobase editor of any one of the previous claims, wherein the nucleobase editor has at least 50% base editing activity as compared to a nucleobase editor comprising the sequence of SEQ ID NO: 3.

34. The nucleobase editor of any one of the previous claims, wherein the nucleobase editor has at least 60% base editing activity as compared to a nucleobase editor comprising the sequence of SEQ ID NO: 3.

35. The nucleobase editor of any one of the previous claims, wherein the nucleobase editor has at least 70% base editing activity as compared to a nucleobase editor comprising the sequence of SEQ ID NO: 3.

36. The nucleobase editor of any one of the previous claims, wherein the nucleobase editor has at least 80% base editing activity as compared to a nucleobase editor comprising the sequence of SEQ ID NO: 3.

37. The nucleobase editor of any one of the previous claims, wherein the nucleobase editor has at least 90% base editing activity as compared to a nucleobase editor comprising the sequence of SEQ ID NO: 3.

38. The nucleobase editor of any one of claims 33-37, wherein the base editing activity is assessed using a polynucleotide comprising the nucleotide sequence of SEQ ID NO: 4.

39. The nucleobase editor of claim 38, wherein the base editing activity is assessed at position A5, A8 or A13 of SEQ ID NO: 4.

40. A truncated nucleobase editor comprising a nucleotide deaminase, a Cas9 nuclease, and a linker between the deaminase and Cas9 nuclease, wherein the nucleobase editor is between 1175-1249 amino acids in length, wherein:
a) the nucleotide deaminase lacks the amino acids corresponding to the first 1, 2, 3, 4, 5, 6, 7, or 8 amino acids of SEQ ID NO: 1 and/or the nucleotide deaminase lacks the amino acids corresponding to the last 1, 2, 3, 4, 5, 6, 7, 8, 9, or 10 amino acids of SEQ ID NO: 1;
b) the Cas9 nuclease lacks one or more amino acids from the range of amino acids corresponding to amino acids 1-4, 1-6, 77-85, 124-126, 124-129, 124-143, 124-145, 131-134, 135-138, 122-129, 505-622, 687-688, 716-720, 718-719, 718-730, 765, and/or 865-873 of SEQ ID NO: 2;
c) the linker between the deaminase and Cas9 nuclease is selected from the group of SEQ ID NO: 11, SEQ ID NO: 12, and SEQ ID NO: 13;
d) the nucleobase editor has at least 50%, 60%, 70%, 80%, or 90% base editing activity as compared to a nucleobase editor comprising the sequence of SEQ ID NO: 3;
e) the nucleobase editor further comprises:
i. one or more nuclear localization signals (NLSs), wherein the one or more NLSs are selected from a c-myc NLS (e.g., an NLS comprising the amino acid sequence of SEQ ID NO: 5), an SV40 NLS (e.g., an NLS comprising the amino acid sequence of SEQ ID NO: 7), and/or a nucleoplasmin NLS (e.g., an NLS comprising the amino acid sequence of SEQ ID NO: 9);
ii. the one or more NLSs are fused to the nucleobase editor optionally through one or more linkers;
iii. wherein the one or more NLSs and one or more linkers fusing the one or more NLSs to the nucleobase editor are collectively no more than 32 amino acids; and
f) the amino acid sequence which encodes the nucleobase editor is selected from the group SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, and SEQ ID NO: 56.

41. The truncated nucleobase editor of claim 40, wherein the truncated nucleobase editor has substantially the same base editing activity as compared to a nucleobase editor comprising the sequence of SEQ ID NO: 3.

42. A protein comprising a Cas9 protein, wherein the Cas9 protein is between 1010-1052, 1010-1040, 1010-1025, 1010-1015, 1020-1052, 1020-1040, 1020-1030, 1030-1052, 1030-1040, 1030-1035, 1035-1046, 1040-1052, 1040-1046, 1045-1052, or 1045-1048 amino acids in length.

43. A protein comprising a Cas9 protein, wherein the Cas9 protein comprises an amino acid sequence that is at least 80%, 85%, 90%, 91%, 92%, 93%, 94%, 95%, 96%, 97%, 98%, 99% or 100% identical to the amino acid sequence of SEQ ID NO: 2, or a functional fragment SEQ ID NO: 2.

44. The protein of claim 42 or 43, wherein the Cas9 protein lacks one or more amino acids from the range of amino acids corresponding to amino acids 1-4, 1-6, 77-85, 124-126, 124-129, 124-143, 124-145, 131-134, 135-138, 122-129, 505-622, 687-688, 716-720, 718-719, 718-730, 765, and/or 865-873 of SEQ ID NO: 2.

45. The protein of any one of claims 42-44, wherein the Cas9 protein lacks the amino acids corresponding to amino acids 1-4, 1-6, 77-85, 124-126, 124-129, 124-143, 124-145, 131-134, 135-138, 122-129, 505-622, 687-688, 716-720, 718-719, 718-730, 765, and/or 865-873 of SEQ ID NO: 2.

46. The protein of any one of claims 42-45, wherein the Cas9 protein lacks the amino acids corresponding to amino acids:
a) 718-719 and 765 of SEQ ID NO: 2,
b) 718-719 and 687-688 of SEQ ID NO: 2,
c) 716-720 and 687-688 of SEQ ID NO: 2,
d) 1-4 and 718-730 of SEQ ID NO: 2,
e) 1-4, 687-688, and 716-720 of SEQ ID NO: 2,
f) 1-4 and 124-145 of SEQ ID NO: 2, or
g) 1-4, 505-622, and 718-730 of SEQ ID NO: 2.

47. The protein of any one of claims 42-46, wherein the Cas9 protein does not lack one or more amino acids from the range of amino acids corresponding to amino acids:
k) 1-8 of SEQ ID NO: 2,
l) 1-10 of SEQ ID NO: 2,
m) 81-94 of SEQ ID NO: 2,
n) 163-164 of SEQ ID NO: 2,
o) 181-184 of SEQ ID NO: 2,
p) 244-424 of SEQ ID NO: 2,
q) 714-769 of SEQ ID NO: 2,
r) 736-742 of SEQ ID NO: 2,
s) 978-979 of SEQ ID NO: 2, and/or
t) 1039-1052 of SEQ ID NO: 2.

48. The protein of any one of claims 42-47, wherein the Cas9 protein does not lack the amino acid ranges corresponding to amino acids:
k) 1-8 of SEQ ID NO: 2,
l) 1-10 of SEQ ID NO: 2,
m) 81-94 of SEQ ID NO: 2,
n) 163-164 of SEQ ID NO: 2,
o) 181-184 of SEQ ID NO: 2,
p) 244-424 of SEQ ID NO: 2,
q) 714-769 of SEQ ID NO: 2,
r) 736-742 of SEQ ID NO: 2,
s) 978-979 of SEQ ID NO: 2, and/or
t) 1039-1052 of SEQ ID NO: 2.

49. The protein of any one of claims 42-49, wherein the Cas9 protein lacks one or more amino acids from the amino acid ranges corresponding to amino acids 81-94, 123-145, 244-424, 714-769, and/or 717-730.

50. The protein of claim 49, wherein the Cas9 protein lacks the amino acids corresponding to amino acids 81-94, 123-145, 244-424, 714-769, and/or 717-730.

51. The protein of any one of claims 42-50, wherein the Cas9 protein is fused to a heterologous moiety.

52. The protein of claim 51, wherein the heterologous moiety is a deaminase.

53. The protein of claim 51, wherein the heterologous moiety is a protein useful for gene silencing (e.g., a KRAB domain, Dnmt3A, and/or Dnmt3L protein domains).

54. The protein of claim 51, wherein the heterologous moiety is a DNA methyltransferase, e.g., Dnmt1, Dnmt3A, or Dnmt3B.

55. The protein of claim 51, wherein the heterologous moiety is a regulatory factor of a factor of DNA methyltransferase (e.g., a catalytically inactive regulatory factor of DNA methyltransferase (e.g., Dnmt3L)).

56. The protein of claim 51, wherein the heterologous moiety is a protein useful for reversing gene silencing (e.g., a TET1 DNA demethylase catalytic domain).

57. The protein of claim 51, wherein the heterologous moiety is a transcriptional repressor.

58. The protein of claim 51, wherein the heterologous moiety is a transcriptional activator.

59. A nucleic acid encoding a nucleobase editor of any one of the previous claims.

60. The nucleic acid of claim 59, further comprising inverted terminal repeats (ITRs).

61. The nucleic acid of claim 59 or 60, further comprising a sgRNA.

62. The nucleic acid of any one of claims 59-61, further comprising promoter sequences.

63. The nucleic acid of any one of claims 59-62, wherein the nucleic acid is less than 5 kb.

64. The nucleic acid of any one of claims 60-62, wherein the nucleic acid from ITR to ITR, including the ITR, is less than 5 kb.

65. The nucleic acid of any one of claims 59-62, wherein the nucleic acid is less than 4.9 kb.

66. The nucleic acid of any one of claims 59-62, wherein the nucleic acid from ITR to ITR, including the ITR, is less than 4.9 kb.

67. The nucleic acid of any one of claims 59-62, wherein the nucleic acid is less than 4.85 kb.

68. The nucleic acid of any one of claims 60-62, wherein the nucleic acid from ITR to ITR, including the ITR, is less than 4.85 kb.

69. The nucleic acid of any one of claims 59-62, wherein the nucleic acid is less than 4.8 kb.

70. The nucleic acid of any one of claims 60-62, wherein the nucleic acid from ITR to ITR, including the ITR, is less than 4.8 kb.

71. A vector comprising a nucleic acid of any one of claims 59-70.

72. The vector of claim 71, wherein the vector is selected from the group of adeno-associated virus, adenovirus, lentivirus, bacteriophage, and virus-like particles.

73. The vector of claim 71 or 72, wherein the vector is an adeno-associated virus.

74. An isolated cell comprising the composition of any one of the previous claims.

75. A method of editing a genomic sequence in a cell comprising administering to the cell the protein of any one of claims 1-58 or the nucleic acid of any one of claims 59-70 or the vector of any one of claims 71-73.

76. The method of claim 75, wherein the method further comprises administering: a) a gRNA targeting a sequence in the cell, or b) a nucleic acid encoding the gRNA.


SEQUENCE LISTING (81 sequences)

SEQ ID NO: 1 - protein, 166 aa, synthetic construct
  REGION 1..166: note = TadA
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 2 - protein, 1052 aa, synthetic construct
  REGION 1..1052: note = nSaCas9
KRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 3 - protein, 1250 aa, synthetic construct
  REGION 1..1250: note = WT nucleobase editor
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 4 - DNA, 22 bp, synthetic construct
  misc_feature 1..22: note = Activity Assessment Polynucleotide
  source 1..22: mol_type = other DNA
atgcattaactgaaaatggtca

SEQ ID NO: 5 - protein, 12 aa, synthetic construct
  REGION 1..12: note = c-myc NLS
PAAKKKKLDIKI

SEQ ID NO: 6 - protein, 4 aa, synthetic construct
  REGION 1..4: note = NLS Linker
GSVD

SEQ ID NO: 7 - protein, 7 aa, synthetic construct
  REGION 1..7: note = SV40 NLS
PKKKRKV

SEQ ID NO: 8 - protein, 15 aa, synthetic construct
  REGION 1..15: note = NLS Linker
TGGGPGGGAAAGSGS

SEQ ID NO: 9 - protein, 16 aa, synthetic construct
  REGION 1..16: note = nucleoplasmin NLS
KRPAATKKAGQAKKKK

SEQ ID NO: 10 - protein, 4 aa, synthetic construct
  REGION 1..4: note = NLS Linker
GSGS

SEQ ID NO: 11 - skipped

SEQ ID NO: 12 - protein, 5 aa, synthetic construct
  REGION 1..5: note = Linker
PAPAP

SEQ ID NO: 13 - protein, 7 aa, synthetic construct
  REGION 1..7: note = Linker
PAPAPAP

SEQ ID NO: 14 - protein, 1223 aa, synthetic construct
  REGION 1..1223: note = (pBE0015)
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINPAPAPKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 15 - protein, 1225 aa, synthetic construct
  REGION 1..1225: note = (pBE0016)
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINPAPAPAPKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 16 - protein, 1221 aa, synthetic construct
  REGION 1..1221: note = (pBE0119)
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINPAPKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 17 - protein, 1221 aa, synthetic construct; identical to SEQ ID NO: 16
  REGION 1..1221: note = (pBE0019)

SEQ ID NO: 18 - protein, 1246 aa, synthetic construct
  REGION 1..1246: note = (pBE0020)
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSGGSSGGSSGSETPGTSESATPESSGGSSGGSKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 19 - protein, 1242 aa, synthetic construct
  REGION 1..1242: note = (pBE0021)
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQSGGSSGGSSGSETPGTSESATPESSGGSSGGSKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 20 - protein, 1244 aa, synthetic construct
  REGION 1..1244: note = (pBE0057)
HEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 21 - protein, 1242 aa, synthetic construct
  REGION 1..1242: note = (pBE0058)
YWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 22 - protein, 1244 aa, synthetic construct
  REGION 1..1244: note = (pBE0059)
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKSGGSSGGSSGSETPGTSESATPESSGGSSGGSKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 23 - protein, 1247 aa, synthetic construct
  REGION 1..1247: note = (pBE0022)
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 24 - protein, 1244 aa, synthetic construct
  REGION 1..1244: note = (pBE0024)
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 25 - protein, 1246 aa, synthetic construct
  REGION 1..1246: note = (pBE0025)
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNEEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 26 - protein, 1246 aa, synthetic construct
  REGION 1..1246: note = (pBE0026)
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 27 - protein, 1237 aa, synthetic construct
  REGION 1..1237: note = (pBE0029)
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 28 - protein, 1228 aa, synthetic construct
  REGION 1..1228: note = (pBE0038)
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 29 - protein, 1241 aa, synthetic construct
  REGION 1..1241: note = (pBE0040)
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 30 - protein, 1236 aa, synthetic construct
  REGION 1..1236: note = (pBE0042)
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLY

SEQ ID NO: 31 - protein, 1246 aa, synthetic construct
  REGION 1..1246: note = (pBE0043)
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 32 - protein, 1215 aa, synthetic construct
  REGION 1..1215: note = (pBE0060)
FSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQPAPAPKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 33 - protein, 1208 aa, synthetic construct
  REGION 1..1208: note = (pBE0061)
FSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQPAPAPKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKFKKERNKGYKHHAEDALIIANADFIFKDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 34 - protein, 1207 aa, synthetic construct
  REGION 1..1207: note = (pBE0105)
FSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQPAPAPILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 35 - protein, 1207 aa, synthetic construct
  REGION 1..1207: note = (pBE0107)
YWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQPAPAPILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 36 - protein, 1209 aa, synthetic construct
  REGION 1..1209: note = (pBE0108)
YWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKPAPAPKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 37 - protein, 1205 aa, synthetic construct
  REGION 1..1205: note = (pBE0109)
YWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKPAPAPILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 38 - protein, 1207 aa, synthetic construct
  REGION 1..1207: note = (pBE0110)
YWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQPAPAPKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 39 - protein, 1238 aa, synthetic construct
  REGION 1..1238: note = (pBE0116)
HALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 40 - protein, 1240 aa, synthetic construct
  REGION 1..1240: note = (pBE0117)
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNSGGSSGGSSGSETPGTSESATPESSGGSSGGSKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 41 - protein, 1238 aa, synthetic construct
  REGION 1..1238: note = (pBE0118)
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVSGGSSGGSSGSETPGTSESATPESSGGSSGGSKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 42 - protein, 1244 aa, synthetic construct
  REGION 1..1244: note = (pBE0120)
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 43 - protein, 1242 aa, synthetic construct
  REGION 1..1242: note = (pBE0121)
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 44 - protein, 1240 aa, synthetic construct
  REGION 1..1240: note = (pBE0122)
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 45 - protein, 1192 aa, synthetic construct
  REGION 1..1192: note = (pBE0123)
YWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKPAPAPILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 46 - protein, 1198 aa, synthetic construct
  REGION 1..1198: note = (pBE0124)
YWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKPAPAPILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKFKKERNKGYKHHAEDALIIANADFIFKDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 47 - protein, 1203 aa, synthetic construct
  REGION 1..1203: note = (pBE0125)
YWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKPAPILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 48 - protein, 1183 aa, synthetic construct
  REGION 1..1183: note = (pBE0126)
YWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKPAPAPILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 49 - protein, 1170 aa, synthetic construct
  REGION 1..1170: note = (pBE0127)
YWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKPAPAPILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 50 - protein, 1242 aa, synthetic construct
  REGION 1..1242: note = (Del3)
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 51 - protein, 1069 aa, synthetic construct
  REGION 1..1069: note = (Del4)
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 52 - protein, 1241 aa, synthetic construct
  REGION 1..1241: note = (Del5)
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSKRNYILGLAIGITSVGYGIIDYETRDVIDAGVRLFKEANVENNEGRRSKRGARRLKRRRRHRIQRVKKLLFDYNLLNPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 53 - protein, 1247 aa, synthetic construct
  REGION 1..1247: note = (Del6)
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWFKKERNKGYKHHAEDALIIANADFIFKEWLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 54 - protein, 1248 aa, synthetic construct
  REGION 1..1248: note = (Del7)
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 55 - protein, 1246 aa, synthetic construct
  REGION 1..1246: note = (Del8)
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKFKKERNKGYKHHAEDALIIANADFIFKEWLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 56 - protein, 1243 aa, synthetic construct
  REGION 1..1243: note = (Del9)
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKFKKERNKGYKHHAEDALIIANADFIFKDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 57 - protein, 1248 aa, synthetic construct
  REGION 1..1248: note = (pBE0027)
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 58 - protein, 1246 aa, synthetic construct
  REGION 1..1246: note = pBE0028
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 59 - protein, 1194 aa, synthetic construct
  REGION 1..1194: note = Del2
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 60 - protein, 1248 aa, synthetic construct
  REGION 1..1248: note = pBE0031
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELVIGVNNDLLNRIEVNMIDITYREYLENMNDKRPPRIIKTIASKTQSIKKYSTDILGNLYEVKSKKHPQIIKKG

SEQ ID NO: 61 - protein, 1236 aa, synthetic construct
  REGION 1..1236: note = pBE0039
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSKRNYILGLAIGITSVGYGIIDYETRDVIDAGVRLFKEANVENNEGRRSKRGARRLKRRRRHRIQRVKKLLFDYNLLTDHSLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 62 - protein, 1243 aa, synthetic construct
  REGION 1..1243: note = pBE0041
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 63 - protein, 1240 aa, synthetic construct
  REGION 1..1240: note = pBE0115
MRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 64 - protein, 1174 aa, synthetic construct
  REGION 1..1174: note = pBE0129
YWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKPAPILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKFKKERNKGYKHHAEDALIIANADFIFKDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 65 - protein, 1053 aa, synthetic construct
  REGION 1..1053: note = SluCas9 protein
NQKFILGLDIGITSVGYGLIDYETKNIIDAGVRLFPEANVENNEGRRSKRGSRRLKRRRIHRLERVKKLLEDYNLLDQSQIPQSTNPYAIRVKGLSEALSKDELVIALLHIAKRRGIHKIDVIDSNDDVGNELSTKEQLNKNSKLLKDKFVCQIQLERMNEGQVRGEKNRFKTADIIKEIIQLLNVQKNFHQLDENFINKYIELVEMRREYFEGPGKGSPYGWEGDPKAWYETLMGHCTYFPDELRSVKYAYSADLFNALNDLNNLVIQRDGLSKLEYHEKYHIIENVFKQKKKPTLKQIANEINVNPEDIKGYRITKSGKPQFTEFKLYHDLKSVLFDQSILENEDVLDQIAEILTIYQDKDSIKSKLTELDILLNEEDKENIAQLTGYTGTHRLSLKCIRLVLEEQWYSSRNQMEIFTHLNIKPKKINLTAANKIPKAMIDEFILSPVVKRTFGQAINLINKIIEKYGVPEDIIIELARENNSKDKQKFINEMQKKNENTRKRINEIIGKYGNQNAKRLVEKIRLHDEQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 66 - protein, 1057 aa, synthetic construct
  REGION 1..1057: note = sRGN1
MNQKFILGLDIGITSVGYGLIDYETKNIIDAGVRLFPEANVENNEGRRSKRGSRRLKRRRIHRLDRVKHLLAEYDLLDLTNIPKSTNPYQTRVKGLNEKLSKDELVIALLHIAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGKGSPFGWEGNIKKWFEQMMGHCTYFPEELRSVKYSYSAELFNALNDLNNLVITRDEDAKLNYGEKFQIIENVFKQKKTPNLKQIAIEIGVHETEIKGYRVNKSGTPEFTEFKLYHDLKSIVFDKSILENEAILDQIAEILTIYQDEQSIKEELNKLPEILNEQDKAEIAKLIGYNGTHRLSLKCIHLINEELWQTSRNQMEIFNYLNIKPNKVDLSEQNKIPKDMVNDFILSPVVKRTFIQSINVINKVIEKYGIPEDIIIELARENNSDDRKKFINNLQKKNEATRKRINEIIGQTGNQNAKRIVEKIRLHDQQEGKCLYSLKDIPLEDLLRNPNNYDIDHIIPRSVSFDDSMHNKVLVRREQNAKKNNQTPYQYLTSGYADIKYSVFKQHVLNLAENKDRMTKKKREYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 67 - protein, 1053 aa, synthetic construct
  REGION 1..1053: note = sRGN2
MNQKFILGLDIGITSVGYGLIDYETKNIIDAGVRLFPEANVENNEGRRSKRGSRRLKRRRIHRLERVKSLLSEYKIISGLAPTNNQPYNIRVKGLTEQLTKDELAVALLHIAKRRGIHKIDVIDSNDDVGNELSTKEQLNKNSKLLKDKFVCQIQLERMNEGQVRGEKNRFKTADIIKEIIQLLNVQKNFHQLDENFINKYIELVEMRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTEFKLYHDLKSVLFDQSILENEDVLDQIAEILTIYQDKDSIKSKLTELDILLNEEDKENIAQLTGYNGTHRLSLKCIRLVLEEQWYSSRNQMEIFTHLNIKPKKINLTAANKIPKAMIDEFILSPVVKRTFIQSINVINKVIEKYGIPEDIIIELARENNSDDRKKFINNLQKKNEATRKRINEIIGQTGNQNAKRIVEKIRLHDQQEGKCLYSLESIALMDLLNNPQNYEVDHIIPRSVAFDNSIHNKVLVKQIENSKKGNRTPYQYLNSSDAKLSYNQFKQHILNLSKSKDRISKKKKDYLLEERDINKFEVQKEFINRNLVDTRYATRELTSYLKAYFSANNMDVKVKTINGSFTNHLRKVWRFDKYRNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 68 - protein, 1057 aa, synthetic construct
  REGION 1..1057: note = sRGN3
MNQKFILGLDIGITSVGYGLIDYETKNIIDAGVRLFPEANVENNEGRRSKRGSRRLKRRRIHRLERVKLLLTEYDLINKEQIPTSNNPYQIRVKGLSEILSKDELAIALLHLAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 69 - protein, 1057 aa, synthetic construct
  REGION 1..1057: note = sRGN3.1
MNQKFILGLDIGITSVGYGLIDYETKNIIDAGVRLFPEANVENNEGRRSKRGSRRLKRRRIHRLERVKLLLTEYDLINKEQIPTSNNPYQIRVKGLSEILSKDELAIALLHLAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKNYRFDVYLTEKGYKFVTIAYLNVFKKDNYYYIPKDKYQELKEKKKIKDTDQFIASFYKNDLIKLNGDLYKIIGVNSDDRNIIELDYYDIKYKDYCEINNIKGEPRIKKTIGKKTESIEKFTTDVLGNLYLHSTEKAPQLIFKRGL

SEQ ID NO: 70 - protein, 1057 aa, synthetic construct
  REGION 1..1057: note = sRGN3.2
MNQKFILGLDIGITSVGYGLIDYETKNIIDAGVRLFPEANVENNEGRRSKRGSRRLKRRRIHRLERVKLLLTEYDLINKEQIPTSNNPYQIRVKGLSEILSKDELAIALLHLAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKFSHRVDKKPNRQLINDTLYSTRMKDEHDYIVQTITDIYGKDNTNLKKQFNKNPEKFLMYQNDPKTFEKLSIIMKQYSDEKNPLAKYYEETGEYLTKYSKKNNGPIVKKIKLLGNKVGNHLDVTNKYENSTKKLVKLSIKNYRFDVYLTEKGYKFVTIAYLNVFKKDNYYYIPKDKYQELKEKKKIKDTDQFIASFYKNDLIKLNGDLYKIIGVNSDDRNIIELDYYDIKYKDYCEINNIKGEPRIKKTIGKKTESIEKFTTDVLGNLYLHSTEKAPQLIFKRGL

SEQ ID NO: 71 - protein, 1057 aa, synthetic construct
  REGION 1..1057: note = sRGN3.3
MNQKFILGLDIGITSVGYGLIDYETKNIIDAGVRLFPEANVENNEGRRSKRGSRRLKRRRIHRLERVKLLLTEYDLINKEQIPTSNNPYQIRVKGLSEILSKDELAIALLHLAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTSYLKAYFSANNMDVKVKTINGSFTNHLRKVWRFDKYRNHGYKHHAEDALIIANADFLFKENKKLQNTNKILEKPTIENNTKKVTVEKEEDYNNVFETPKLVEDIKQYRDYKFSHRVDKKPNRQLINDTLYSTRMKDEHDYIVQTITDIYGKDNTNLKKQFNKNPEKFLMYQNDPKTFEKLSIIMKQYSDEKNPLAKYYEETGEYLTKYSKKNNGPIVKKIKLLGNKVGNHLDVTNKYENSTKKLVKLSIKNYRFDVYLTEKGYKFVTIAYLNVFKKDNYYYIPKDKYQELKEKKKIKDTDQFIASFYKNDLIKLNGDLYKIIGVNSDDRNIIELDYYDIKYKDYCEINNIKGEPRIKKTIGKKTESIEKFTTDVLGNLYLHSTEKAPQLIFKRGL

SEQ ID NO: 72 - protein, 1054 aa, synthetic construct
  REGION 1..1054: note = sRGN4
MNQKFILGLDIGITSVGYGLIDYETKNIIDAGVRLFPEANVENNEGRRSKRGSRRLKRRRIHRLERVKKLLEDYNLLDQSQIPQSTNPYAIRVKGLSEALSKDELVIALLHIAKRRGIHNINVSSEDEDASNELSTKEQINRNNKLLKDKYVCEVQLQRLKEGQIRGEKNRFKTTDILKEIDQLLKVQKDYHNLDIDFINQYKEIVETRREYFEGPGKGSPYGWEGDPKAWYETLMGHCTYFPDELRSVKYAYSADLFNALNDLNNLVIQRDGLSKLEYHEKYHIIENVFKQKKKPTLKQIANEINVNPEDIKGYRITKSGKPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 73 - protein, 71 aa, synthetic construct
  REGION 1..71: note = KRAB domain
DAKSLTAWSRTLVTFKDVFVDFTREEWKLLDTAQQIVYRNVMLENYKNLVSLGYQLTKPDVILRLEKGEEP

SEQ ID NO: 74 - protein, 301 aa, synthetic construct
  REGION 1..301: note = Dnmt3A
NHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 75 - protein, 215 aa, synthetic construct
  REGION 1..215: note = Dnmt3L
MGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPL

SEQ ID NO: 76 - protein, 1133 aa, synthetic construct
  REGION 1..1133: note = ABE8e_SaCas9_delta_E506-K623
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 77 - protein, 1116 aa, synthetic construct
  REGION 1..1116: note = ABE8e_SaCas9_delta_K2-Y5_K719-Q731_E506-K623
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEYLLEERDINRFSVQKDFINRNLVDTRYATRGLMNLLRSYFRVNNLDVKVKSINGGFTSFLRRKWKFKKERNKGYKHHAEDALIIANADFIFKEWMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 78 - protein, 32 aa, synthetic construct
  REGION 1..32: note = Linker
SGGSSGGSSGSETPGTSESATPESSGGSSGGS

SEQ ID NO: 79 - protein, 4 aa, synthetic construct
  REGION 1..4: note = Linker
GGSS

SEQ ID NO: 80 - protein, 6 aa, synthetic construct
  REGION 1..6: note = Linker
GGSSGG

SEQ ID NO: 81 - protein, 8 aa, synthetic construct
  REGION 1..8: note = Linker
SGGSSGGS